(19) United States
(12) Patent Application Publication
(10) Pub. No.: US 2024/0197841 A1
(43) Pub. Date: Jun. 20, 2024

(54) FORMULATIONS OF L-ASPARAGINASE

(71) Applicant: Jazz Pharmaceuticals Ireland Ltd., Dublin (IE)

(72) Inventors: Sekhar Kanapuram, Dublin (IE); Haripada Maity, Dublin (IE); Guifeng Jiang, Dublin (IE); Roja Narwal, Dublin (IE)

(73) Assignee: Jazz Pharmaceuticals Ireland Ltd., Tokyo (JP)

(21) Appl. No.: 18/285,209

(22) PCT Filed: Apr. 5, 2022
(86) PCT No.: PCT/US2022/071562

§ 371 Date: Sep. 29, 2023

Related U.S. Application Data
(60) Provisional application No. 63/171,429, filed on Apr. 6, 2021.

(30) Foreign Application Priority Data
Mar. 29, 2021 (JP) 2021-055393

(51) Int. Cl. A61K 38/50; A61K 9/08; A61K 45/06; A61K 47/02; A61K 47/12; A61K 47/26

(52) U.S. Cl. CPC A61K 38/50; A61K 9/08; A61K 45/06; A61K 47/02; A61K 47/12; A61K 47/26

(57) ABSTRACT

Provided herein are stable, aqueous, non-lyophilized formulations of L-asparaginase. Also provided herein are methods of using such formulations in the treatment of one or more diseases, disorders, or conditions (for example, cancers such as Acute Lymphoblastic Leukemia (ALL) or Lymphoblastic Lymphoma (LBL)) that are treatable by asparagine depletion.

### CROSS-REFERENCE TO RELATED APPLICATION

This application claims priority to and benefit of U.S. Provisional Patent Application No. 63/171,429, filed Apr. 6, 2021, the disclosure of which is hereby incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 210462000340SEQLIST.TXT, date recorded: Apr. 5, 2022, size: 3,096 bytes).

### BACKGROUND OF THE INVENTION

Proteins with L-asparagine aminohydrolase activity, commonly known as L-asparaginases, have successfully been used for the treatment of various diseases that are potentially fatal, including cancers such as Acute Lymphoblastic Leukemia (ALL) and Lymphoblastic Lymphoma (LBL), for which children constitute a large proportion of patients stricken with these diseases.

L-asparaginases of bacterial origin have a high immunogenic and antigenic potential. Currently on the market as first line treatment are E. coli derived L-asparaginase and pegaspargase. These products can provoke adverse hypersensitivity reactions including allergic reaction, silent inactivation, and anaphylactic shock in patients. Patients who experience hypersensitivity reactions to these products often have to stop treatment, resulting in poorer prognosis and survival rates. These patients have turned to Erwinaze® after experiencing hypersensitivity reactions. Erwinaze® has been plagued by supply issues for years: reportedly, it can take 9 months to prepare (See, e.g., “Saving Ava: When a cancer drug shortage imperiled a toddler, her mom got to work”, Stat News, Karen Weintraub (Oct. 31, 2016); Asparaginase Erwinia Chrysanthemi Drug Shortage, Drugs.com). Even today the issues persist and Erwinase® shortages are common. The FDA has issued warning letters to the manufacturer stating that “changes in the source material or cell line have a substantial potential to adversely affect the identity, strength, quality, purity or potency of Erwinase®.”

There is a need for immunogenically non-cross reactive treatment options. A L-asparaginase (like L-asparaginase recombinantly produced in Pseudomonas fluorescens) with no immunological cross-reactivity to E. coli-derived asparaginase would address a significant medical need (as a component of a multi-agent chemotherapeutic regimen) for patients with ALL/Lymphoblastic Lymphoma (LBL), by helping to ensure availability of an asparaginase for patients who have developed hypersensitivity to E. coli-derived asparaginase.

Currently, Erwinase® is supplied as a sterile, lyophilized, white powder in vials. Each vial contains 10,000 International Units of asparaginase Erwinia chrysanthemi, and the following inactive ingredients: glucose monohydrate (5.0 mg), sodium chloride (0.5 mg). The availability of non-lyophilized formulations removes the need for rehydration before administration to a subject, e.g., a human. To that end, Oncaspar®, an alternative, non-lyophilized treatment option, is supplied as a clear, colorless, preservative-free, isotonic sterile solution in phosphate-buffered saline, pH 7.3. Each milliliter contains 750±150 International Units of pegaspargase, dibasic sodium phosphate, USP (5.58 mg), monobasic sodium phosphate, USP, (1.20 mg) and sodium chloride, USP (8.50 mg) in water for injection, USP. In addition, ASPARLAS®, another non-lyophilized alternative, is supplied as a clear, colorless, preservative-free, isotonic sterile solution in phosphate-buffered saline, pH 7.3, that requires dilution prior to intravenous infusion. Each vial of ASPARLAS® contains 3,750 units in 5 mL of solution. Each milliliter contains 750 units of calaspargase pegol-mknl; dibasic sodium phosphate, USP (5.58 mg); monobasic sodium phosphate, USP (1.20 mg); and sodium chloride, USP (8.50 mg) in water for injection, USP. However, though non-lyophilized formulations (such as Oncaspar® and ASPARLAS®) exist, there are issues with stability: for example, the existing formulations must be used within 48 hours if stored at room temperature.

Thus, there is a need in the art for non-lyophilized, stable, aqueous formulations of L-asparaginase.

### BRIEF SUMMARY OF THE INVENTION

Provided herein are aqueous, non-lyophilized formulations of L-asparaginase. In some aspects, such formulations may find use in the treatment of one or more diseases, disorders, or conditions treatable by asparagine depletion, including, for example, cancers such as Acute Lymphoblastic Leukemia (ALL) and Lymphoblastic Lymphoma (LBL), including relapsed ALL and relapsed LBL.

In one aspect, provided herein is an aqueous, non-lyophilized formulation, comprising:
(i) an L-asparaginase, wherein the L-asparaginase comprises four monomer units, wherein each monomer unit has an amino acid sequence that is at least about 70% identical to SEQ ID NO: 1; and
(ii) one or more stabilizers, or one or more buffers, or any combination thereof.

In some embodiments, the one or more stabilizers comprise one or more disaccharides, one or more sorbitols, one or more amino acids, or any combination thereof. In some embodiments, the one or more disaccharides comprise trehalose, sucrose, or any combination thereof. In some embodiments, the one or more disaccharides comprise trehalose. In some embodiments, the one or more buffers are substantially free of amino acid. In some embodiments, the one or more buffers comprise a phosphate buffer, an acetate buffer, or any combination thereof. In some embodiments, the one or more buffers comprise a phosphate buffer. In some embodiments, the one or more buffers comprise sodium phosphate. In some embodiments, the sodium phosphate is sodium phosphate dibasic anhydrous, sodium phosphate monobasic monohydrate, or a combination thereof. In some embodiments, the formulation further comprises sodium chloride. In some embodiments, the formulation further comprises one or more excipients. In some embodiments, the one or more excipients comprise polysorbate 80, polysorbate 20, poloxamer 188, or any combination thereof. In some embodiments, the formulation has a pH of between about 4.0 and about 8.5. In some embodiments, the L-asparaginase is present at a concentration of about 20 mg/mL. In some embodiments, the L-asparaginase is non-PEGylated and non-PASylated.

In some embodiments, the formulation comprises less than about 0.6% low-molecular-weight (LMW) species after storage at 40° C. for two months. In some embodiments, the formulation comprises less than about 0.6% low-molecular-weight (LMW) species after storage at 37° C. for one week. In some embodiments, the formulation comprises less than 2% high-molecular-weight (HMW) species after storage at 40° ° C. for two months.

In one aspect, provided herein is an aqueous, non-lyophilized formulation, comprising:
(i) an L-asparaginase, wherein the L-asparaginase comprises four monomer units, wherein each monomer unit has an amino acid sequence that is at least about 95% identical to SEQ ID NO: 1;
(ii) one or more disaccharides, wherein the one or more disaccharides comprise trehalose, sucrose, or any combination thereof; and
(iii) one or more buffers, wherein the one or more buffers are substantially free of amino acid, wherein the formulation comprises less than about 5% low-molecular-weight (LMW) species after storage at 37° ° C. for one week.

In another aspect, provided herein is a method of treating a disease, condition, or disorder that is treatable by asparagine depletion in a subject in need thereof, comprising administering to the subject any of the formulations described herein.

In another aspect, provided herein is a unit dosage form, comprising: (i) any of the formulations described herein; and (ii) one or more pharmaceutically acceptable excipients.

In another aspect, provided herein is a kit, comprising: (i) any of the formulations described herein; and (ii) instructions for treating a disease, condition, or disorder that is treatable by asparagine depletion in a subject in need thereof.

### DETAILED DESCRIPTION

### Definitions

“Subject” refers to mammals, and includes humans and non-human mammals. Examples of individuals include, but are not limited to mice, rats, hamsters, guinea pigs, pigs, rabbits, cats, dogs, goats, sheep, cows, and humans. In some embodiments, subject refers to a human.

The articles “a” and “an” as used herein and in the appended claims are used to refer to one or to more than one (i.e., to at least one) of the grammatical object of the article unless the context clearly indicates otherwise. By way of example, “a buffer” means one buffer or more than one buffer.

As used herein, “about” a parameter or value includes and describes that parameter or value per se. For example, “about X” includes and describes X per se. As used herein, the term “about” modifying, for example, the dimensions, volumes, quantity of an ingredient in a composition, concentrations, process temperature, process time, yields, flow rates, pressures, and like values, and ranges thereof, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 10, 9, 8, 7, 6, 5, 4, 3, 2, or 1 percent or less of the stated reference value.

As used herein, the terms “treatable by depletion of asparagine” and “treatable by asparagine depletion” refers to a disease, condition, or disorder wherein the cells involved in or responsible for the disease, condition, or disorder either lack or have a reduced ability to synthesize L-asparagine. Depletion or deprivation of L-asparagine can be partial or substantially complete (e.g., to levels that are undetectable using methods and apparatus that are known in the art).

The term “comprising the sequence of SEQ ID NO: 1” means that the amino-acid sequence of the protein may not be strictly limited to SEQ ID NO: I but may contain additional amino acids.

“Low-molecular-weight species” or “LMW species”, as used herein, refers to species that have a molecular weight that is lower than that of L-asparaginase. In some embodiments, the LMW species have a molecular weight that is less than about 100%, less than about 90%, less than about 80%, less than about 70%, less than about 60%, less than about 50%, less than about 45%, less than about 40%, less than about 35%, less than about 30%, less than about 25%, less than about 20%, less than about 15%, less than about 10%, less than about 5%, less than about 2%, or less than about 1% of the molecular weight of L-asparaginase. In some embodiments, LMW species are formed by the degradation or decomposition of L-asparaginase, e.g., through hydrolysis of one or more covalent bonds in the L-asparaginase.

“High-molecular-weight species” or “HMW species”, as used herein, refers to species that have a molecular weight that is greater than that of L-asparaginase. In some embodiments, the HMW species have a molecular weight that is greater than about 100%, greater than about 150%, greater than about 200%, greater than about 250%, greater than about 300%, greater than about 350%, greater than about 400%, greater than about 450%, or greater than about 500% of the molecular weight of L-asparaginase.

“Trehalose” refers to a disaccharide having the following structure:

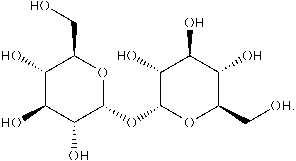 [SMILES: OC[C@H]1O[C@H](O[C@H]2O[C@H](CO)[C@@H](O)[C@H](O)[C@H]2O)[C@H](O)[C@@H](O)[C@@H]1O]

“Sucrose” refers to a disaccharide having the following structure:

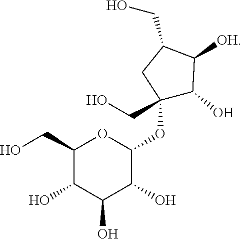 [SMILES: OC[C@H]1C[C@@](CO)(O[C@H]2O[C@H](CO)[C@@H](O)[C@H](O)[C@H]2O)[C@@H](O)[C@@H]1O]

“Polysorbate 80” or “PS-80” refers to an excipient having the following structure:

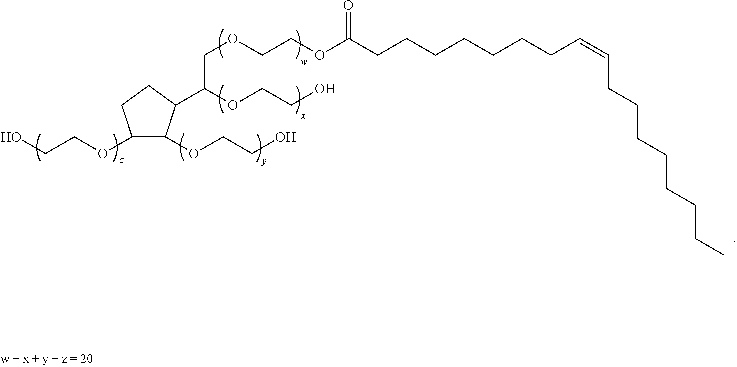

“PEGylated”, as used herein, is used to describe a structure or moiety to which PEG (polyethylene glycol) is conjugated or attached. Any suitable number of PEG units may be attached in any suitable way (e.g., through one or more covalent bonds) and at any suitable location of the structure or moiety in question. For example, a “PEGylated L-asparaginase” indicates an I-asparaginase to which one or more PEG units have been conjugated or attached. Accordingly, a “non-PEGylated” moiety is one to which PEG has not been conjugated or attached.

“PASylation”, as used herein, is used to describe a structure or moiety to which one or more polypeptide units comprising proline, alanine, or serine, or any combination thereof, is conjugated attached. Any suitable number of such polypeptide units may be attached in any suitable way (e.g., through one or more covalent bonds) and at any suitable location of the structure or moiety in question. For example, a “PASylated L-asparaginase” indicates an L-asparaginase to which one or more polypeptide units comprising proline, alanine, or serine, or any combination thereof, have been conjugated or attached. Accordingly, a “non-PASylated” moiety is one to which one or more polypeptide units comprising proline, alanine, or serine, or any combination thereof, has not been conjugated or attached.

“SEQ ID NO: 1” is as follows:

ADKLPNIVILATGGTIAGSAATGTQTTGYKAGALGVDTLINAVPEVKKL
ANVKGEQFSNMASENMTGDVVLKLSQRVNELLARDDVDGVVITHGTDTV
EESAYFLHLTVKSDKPVVFVAAMRPATAISADGPMNLLEAVRVAGDKQS
RGRGVMVVLNDRIGSARYITKTNASTLDTFKANEEGYLGVIIGNRIYYQ
NRIDKLHTTRSVFDVRGLTSLPKVDILYGYQDDPEYLYDAAIQHGVKGI
VYAGMGAGSVSVRGIAGMRKAMEKGVVVIRSTRTGNGIVPPDEELPGLV
SDSLNPAHARILLMLALTRTSDPKVIQEYFHTY.

### Formulations

In one aspect, provided herein is an aqueous, non-lyophilized formulation, comprising: (i) an L-asparaginase, wherein the L-asparaginase comprises four monomer units, wherein each monomer unit has an amino acid sequence that is at least about 70% identical to SEQ ID NO: 1; and (ii) one or more stabilizers, or one or more buffers, or any combination thereof.

In some embodiments, the L-asparaginase formulation comprises one or more stabilizers. In some embodiments, the one or more stabilizers comprise one or more disaccharides, one or more sorbitols, one or more amino acids, or any combination thereof. In some embodiments, the L-asparaginase formulation comprises one or more disaccharides. In some embodiments, the one or more disaccharides comprise trebalose, sucrose, or any combination thereof. In some embodiments, the L-asparaginase formulation comprises one or more buffers. In some embodiments, the one or more buffers, wherein the one or more buffers are substantially free of amino acid. In some embodiments, the L-asparaginase formulation comprises one or more stabilizers and one or more buffers. In some embodiments, the L-asparaginase formulation comprises less than about 0.6% low-molecular-weight (LMW) species after storage at 40° ° C. for two months. In some embodiments, the L-asparaginase formulation comprises less than 2% high-molecular-weight (HMW) species after storage at 40° C. for two months. In some embodiments, the L-asparaginase formulation comprises less than about 0.6% low-molecular-weight (LMW) species and less than 2% high-molecular-weight (HMW) species after storage at 40° ° C. for two months. In some embodiments, the L-asparaginase formulation comprises less than about 0.6% low-molecular-weight (LMW) species after storage at 37° ° C. for one week.

In one aspect, provided herein is an aqueous, non-lyophilized formulation, comprising: (i) an L-asparaginase, wherein the L-asparaginase comprises four monomer units, wherein each monomer unit has an amino acid sequence that is at least about 95% identical to SEQ ID NO: 1; (ii) one or more disaccharides, wherein the one or more disaccharides comprise trehalose, sucrose, or any combination thereof; and (iii) one or more buffers, wherein the one or more buffers are substantially free of amino acid, wherein the formulation comprises less than about 5% low-molecular-weight (LMW) species after storage at 37° C. for one week.

In one aspect, a L-asparaginase in accordance with the disclosure provided herein is an recombinant L-asparaginase. In a further aspect, a L-asparaginase in accordance with the invention described herein is an enzyme with L-asparagine aminohydrolase activity. Such a L-asparaginase's enzymatic activity may include not only deamidation of asparagine to aspartic acid and ammonia, but also deamidation of glutamine to glutamic acid and ammonia.

In some embodiments, a L-asparaginase as disclosed herein is active as a multimer. In some embodiments, the L-asparaginase is an active enzyme as a tetramer. A tetramer is composed of four subunits (also known as monomers). In some embodiments, a L-asparaginase is a tetramer consisting of four identical 35 kD subunits. In some embodiments, the L-asparaginase is a non-disulfide bonded tetrameric therapeutic protein. In a particular embodiment, each of the subunits or monomers of a multimeric L-asparaginase comprises the amino acid sequence of SEQ ID NO: 1.

In a particular embodiment, each of the subunits or monomers of a tetrameric Lasparaginase comprises the amino acid sequence of SEQ ID NO: 1. In another embodiment, the L-asparaginase is from Erwinia chrysanthemi NCPPB 1066 (Genbank Accession No. CAA32884, incorporated herein by reference in its entirety), either with or without signal peptides and/or leader sequences.

In some embodiments, the L-asparaginase is composed of multiple subunits, for example, four subunits or monomers (tetramer). A corresponding modified protein may then, e g., consist of 1 to 20 (or more) peptides conjugated to each of the monomers of that tetramer. In some embodiments, the L-asparaginase comprises a monomer and 1, 2, 3, 4, 5, 6, 7, 8, 9, 10, 11, 12, 13, 14, 15, 16, 17, 18, 19, and 20 (or more) peptides conjugated to each of the L-asparaginase monomers. In a specific embodiment, the L-asparaginase is a multimer comprising multiple subunits or monomers, such as a tetramer, and each of the monomers in that tetramer is conjugated to 1 peptide, resulting in a tetramer comprising 4 conjugated peptides, one for each monomer. In some embodiments, the L-asparaginase is a tetramer comprising 1-4 peptides conjugated to each of the monomers.

In some embodiments, the L-asparaginase is a tetramer comprising 4-20 peptides conjugated to each of the L-monomers. In some embodiments, the Lasparaginase is a tetramer comprising 6-18 peptides conjugated to each of the monomers. In some embodiments, the L-asparaginase is a tetramer comprising 6-18 peptides conjugated to each of the monomers. In some embodiments, the L-asparaginase is a tetramer comprising 10-15 peptides conjugated to each of the monomers.

In one aspect, the invention relates to a modified protein having a L-asparaginase and multiple chemically attached peptide sequences. In a further aspect the length of the peptide sequences are from about 10 to about 100, from about 15 to about 60 or from about 20 to about 40.

Fragments of L-asparaginase, preferably fragments of the L-asparaginase of SEQ ID NO: 1, may be of use in the presently described invention. The term “a fragment of L-asparaginase” (e.g., a fragment of the L-asparaginase of SEQ ID NO: 1) means that the sequence of the L-asparaginase may include fewer amino-acids than in the L-asparaginases exemplified herein (e.g., the L-asparaginase of SEQ ID NO: 1) but still enough amino-acids to confer L-aminohydrolase activity. For example, a “fragment of L-asparaginase” is a fragment that is/consists of at least about 150 or 200 contiguous amino acids of one of the L-asparaginases exemplified herein (e.g. the L-asparaginase of SEQ ID NO: 1) (for example, about 150, 160, 170, 180, 190, 200, 210, 220, 230, 240, 250, 260, 270, 280, 290, 300, 310, 320, 321, 322, 323, 324, 325, or 326 contiguous amino acids) and/or wherein said fragment has up to 50 amino acids deleted from the N-terminus of said L-asparaginases exemplified herein (e.g. the L-asparaginase of SEQ ID NO: 1) (e.g. up to 1, 2, 3, 4, 5, 6, 7, 8, 9, 10, 15, 20, 25, 30, 35, 40, 45, or 50) and/or has up to up to 75 or 100 amino acids deleted from the C-terminus of said L-asparaginases exemplified herein (e.g., the Lasparaginase of SEQ ID NO: 1) (e.g. up to 1, 2, 3, 4, 5, 6, 7, 8, 9, 10, 15, 20, 25, 30, 35, 40, 45, 50, 55, 60, 70, 75, 80, 85, 90, 95 or 100) and/or has deleted amino acids at both the N-terminus and the C-terminus of said L-asparaginases exemplified herein (e.g., the L-asparaginase of SEQ ID NO: 1), wherein the total number of amino acids deleted can be up to 125 or 150 amino acids.

Indeed, a person skilled in the art will understand how to select and design homologous proteins retaining substantially their L-asparaginase activity. Typically, a Nessler assay is used for the determination of L-asparaginase activity according to a method described by Mashburn and Wriston (Mashburn, L., and Wriston, J. (1963) “Tumor Inhibitory Effect of L Asparaginase,” Biochem Biophys Res Commun 12, 50, incorporated herein by reference in its entirety).

It is well known in the art that a polypeptide can be modified by substitution, insertion, deletion and/or addition of one or more amino-acids while retaining its enzymatic activity. The term “one or more amino acids” in this context can refer to one, two, three, four, five, six, seven, eight, nine, ten or more amino acids. For example, substitution of one amino-acid at a given position by a chemically equivalent amino-acid that does not affect the functional properties of a protein is common Substitutions may be defined as exchanges within one of the following groups. Small aliphatic, non-polar or slightly polar residues: Ala, Ser, Thr, Pro, Gly; Polar, negatively charged residues and their amides: Asp, Asn, Glu, Gln; Polar, positively charged residues: His, Arg, Lys, Large aliphatic, non-polar residues: Met, Leu, Ile, Val, Cys; Large aromatic residues: Phe, Tyr, Trp.

Thus, changes that result in the substitution of one negatively charged residue for another (such as glutamic acid for aspartic acid) or one positively charged residue for another (such as lysine for arginine) can be expected to produce a functionally equivalent product.

The positions where the amino-acids are modified and the number of amino-acids that may be modified in the amino-acid sequence are not particularly limited. The skilled artisan is able to recognize the modifications that can be introduced without affecting the activity of the protein. For example, modifications in the N- or C-terminal portion of a protein may be expected not to alter the activity of a protein under certain circumstances. With respect to asparaginases, in particular, much characterization has been done, particularly with respect to the sequences, structures, and the residues forming the active catalytic site. This provides guidance with respect to residues that can be modified without affecting the activity of the enzyme. All known Lasparaginases from bacterial sources have common structural features. All are homotetramers with four active sites between the N- and C-terminal domains of two adjacent monomers (Aghaipour (2001) Biochemistry 40, 5655-5664, incorporated herein by reference in its entirety). All have a high degree of similarity in their tertiary and quaternary structures (Papageorgiou (2008) FEBS J. 275, 4306-4316, incorporated herein by reference in its entirety). The sequences of the catalytic sites of L-asparaginases are highly conserved between Erwinia chrysanthemi, Erwinia carotovora, and E. coli L-asparaginase II (Id). The active site flexible loop contains amino acid residues 14-33, and structural analysis show that Thr15, Thr95, Ser62, Glu63, Asp96, and Alal20 contact the ligand (Id). Aghaipour et al. have conducted a detailed analysis of the four active sites of Erwinia chrysanthemi L-asparaginase by examining high resolution crystal structures of the enzyme complexed with its substrates (Aghaipour (2001) Biochemistry 40, 5655-5664). Kotzia et al. provide sequences for L-asparaginases from several species and subspecies of Erwinia and, even though the proteins have only about 75-77% identity between Erwinia chrysanthemi and Erwinia carotovora, they each still have L-asparaginase activity (Kotzia (2007) J. Biotechnol. 127, 657-669). Moola et al performed epitope mapping studies of Erwinia chrysanthemi 3937 L-asparaginase and were able to retain enzyme activity even after mutating various antigenic sequences in an attempt to reduce immunogenicity of the asparaginase (Moola (1994) Biochem. J. 302, 921-927). In view of the extensive characterization that has been performed on L-asparaginases, one of skill in the art could determine how to make fragments and/or sequence substitutions while still retaining enzyme activity.

More specifically, fragments of the protein of SEQ ID NO: 1 are also comprised within the definition of the protein used in the L-asparaginase of the invention. The term “a fragment of SEQ ID NO: 1” means that the sequence of the polypeptide may include fewer amino-acids than the full-length SEQ ID NO: 1 but retains enough of the protein to confer Laminohydrolase activity. In some embodiments, a L-asparaginase has at least about 80% homology or identity with the protein comprising SEQ ID NO: 1.

In some embodiments, the L-asparaginase of the formulation comprises four monomer units, wherein each monomer unit has an amino acid sequence that is at least about 95% identical to SEQ ID NO: 1. In some embodiments, each monomer unit has an amino acid sequences that is at least about 70%, about 71%, about 72%, about 73%, about 74%, about 75%, about 76%, about 77%, about 78%, about 79%, about 80%, about 81%, about 82%, about 83%, or about 84% identical to SEQ ID NO: 1. In some embodiments, each monomer unit has an amino acid sequences that is at least about 85%, about 86%, about 87%, about 88%, about 89%, about 90% about 91%, about 92%, about 93%, about 94%, about 95%, about 96%, about 97%, about 98%, or about 99% identical to SEQ ID NO: 1

In some embodiments, the L-asparaginase of the formulation is present at a concentration of about 20 mg/mL.

In some embodiments, the L-asparaginase of the formulation is non-PEGylated. In some embodiments, the L-asparaginase of the formulation is non-PASylated. In some embodiments, the L-asparaginase of the formulation is non-PEGylated and non-PASylated.

In some embodiments, in conjunction with embodiments above or below, the L-asparaginase formulation comprises one or more stabilizers. In some embodiments, in conjunction with embodiments above or below, the one or more stabilizers comprise one or more disaccharides, one or more sorbitols, one or more amino acids, or any combination thereof. In some embodiments, in conjunction with embodiments above or below, the L-asparaginase formulation comprises one or more buffers. In some embodiments, in conjunction with embodiments above or below, the one or more buffers comprise acetate, glutamate, citrate, histidine, succinate, phosphate, hydroxymethylaminomethane (e.g., Tris), or any combination thererof. In some embodiments, in conjunction with embodiments above or below, the L-asparaginase formulation comprises one or more surfactants. In some embodiments, in conjunction with embodiments above or below, the one or more surfactants comprise polysorbate 80 (e.g., Tween 80), polysorbate 20 (e.g., Tween 20), poloxamer 188, or any combination thereof. In some embodiments, in conjunction with embodiments above or below, the L-asparaginase formulation comprises one or more polyol (e.g., mannitol and sorbitol), one or more disaccharides (e.g., sucrose and trehalose), and one or more polysaccharides (e.g., dextran 40). In some embodiments, in conjunction with embodiments above or below, the L-asparaginase formulation comprises sodium chloride. In some embodiments, in conjunction with embodiments above or below, the L-asparaginase formulation comprises one or more amino acids (e.g., proline, glycine, and arginie).

In some embodiments, in conjunction with embodiments above or below, the formulation comprises one or more stabilizers at a concentration of between about 50 mM and about 300 mM. In some embodiments, the formulation comprises one or more stabilizers at a concentration of between about 75 mM and about 300 mM, between about 100 mM and about 300 mM, between about 125 mM and about 300 mM, between about 150 mM and about 300 mM, between about 175 mM and about 300 mM, between about 200 mM and about 300 mM, between about 225 mM and about 300 mM, between about 250 mM and about 300 mM, or between about 275 mM and about 300 mM. In some embodiments, the formulation comprises one or more stabilizers at a concentration of between about 50 mM and about 275 mM, between about 50 mM and about 250 mM, between about 50 mM and about 225 mM, between about 50 mM and about 200 mM, between about 50 mM and about 175 mM, between about 50 mM and about 150 mM, between about 50 mM and about 125 mM, between about 50 mM and about 100 mM, or between about 50 mM and about 75 mM. In some embodiments, the formulation comprises one or more stabilizers at a concentration of between about 150 mM and about 275 mM. In some embodiments, the formulation comprises one or more stabilizers at a concentration of between about 150 mM and about 200 mM, between about 150 mM and about 190 mM, between about 150 mM and about 180 mM, between 150 mM and about 170 mM, or between about 160 mM and 150 mM. In some embodiments, the formulation comprises one or more stabilizers at a concentration of between about 160 mM and about 200 mM, between about 170 mM and about 200 mM, between about 180 mM and about 200 mM, or between about 190 mM and about 200 mM. In some embodiments, the formulation comprises one or more stabilizers at a concentration of about 170 mM.

In some embodiments, the one or more disaccharides of the formulation comprise trehalose. In other embodiments, the one or more disaccharides of the formulation comprise sucrose. In some embodiments, the one or more disaccharides of the formulation comprise a combination of trehalose and sucrose.

In some embodiments, the formulation comprises one or more disaccharides at a concentration of between about 50 mM and about 300 mM. In some embodiments, the formulation comprises one or more disaccharides at a concentration of between about 75 mM and about 300 mM, between about 100 mM and about 300 mM, between about 125 mM and about 300 mM, between about 150 mM and about 300 mM, between about 175 mM and about 300 mM, between about 200 mM and about 300 mM, between about 225 mM and about 300 mM, between about 250 mM and about 300 mM, or between about 275 mM and about 300 mM. In some embodiments, the formulation comprises one or more disaccharides at a concentration of between about 50 mM and about 275 mM, between about 50 mM and about 250 mM, between about 50 mM and about 225 mM, between about 50 mM and about 200 mM, between about 50 mM and about 175 mM, between about 50 mM and about 150 mM, between about 50 mM and about 125 mM, between about 50 mM and about 100 mM, or between about 50 mM and about 75 mM. In some embodiments, the formulation comprises one or more disaccharides at a concentration of between about 150 mM and about 275 mM. In some embodiments, the formulation comprises one or more disaccharides at a concentration of between about 150 mM and about 200 mM, between about 150 mM and about 190 mM, between about 150 mM and about 180 mM, between 150 mM and about 170 mM, or between about 160 mM and 150 mM. In some embodiments, the formulation comprises one or more disaccharides at a concentration of between about 160 mM and about 200 mM, between about 170 mM and about 200 mM, between about 180 mM and about 200 mM, or between about 190 mM and about 200 mM. In some embodiments, the formulation comprises one or more disaccharides at a concentration of about 170 mM.

In some embodiments, the formulation comprises trebalose at a concentration of between about 50 mM and about 300 mM. In some embodiments, the formulation comprises one or more disaccharides at a concentration of between about 75 mM and about 300 mM, between about 100 mM and about 300 mM, between about 125 mM and about 300 mM, between about 150 mM and about 300 mM, between about 175 mM and about 300 mM, between about 200 mM and about 300 mM, between about 225 mM and about 300 mM, between about 250 mM and about 300 mM, or between about 275 mM and about 300 mM. In some embodiments, the formulation comprises trehalose at a concentration of between about 50 mM and about 275 mM, between about 50 mM and about 250 mM, between about 50 mM and about 225 mM, between about 50 mM and about 200 mM, between about 50 mM and about 175 mM, between about 50 mM and about 150 mM, between about 50 mM and about 125 mM, between about 50 mM and about 100 mM, or between about 50 mM and about 75 mM. In some embodiments, the formulation comprises trehalose at a concentration of between about 150 mM and about 275 mM. In some embodiments, the formulation comprises trehalose at a concentration of between about 150 mM and about 200 mM, between about 150 mM and about 190 mM, between about 150 mM and about 180 mM, between 150 mM and about 170 mM, or between about 160 mM and 150 mM. In some embodiments, the formulation comprises trehalose at a concentration of between about 160 mM and about 200 mM, between about 170 mM and about 200 mM, between about 180 mM and about 200 mM, or between about 190 mM and about 200 mM. In some embodiments, the formulation comprises trehalose at a concentration of about 170 mM.

In some embodiments, the one or more buffers of the formulation are substantially free of amino acid. In some embodiments, the one or more buffers of the formulation are substantially free of histidine or arginine, or both. In some embodiments, the one or more buffers of the formulation are substantially free of histidine. In some embodiments, the one or more buffers of the formulation comprise a phosphate buffer, an acetate buffer, or any combination thereof. In some embodiments, the one or more buffers comprise a phosphate buffer. In some embodiments, the one or more buffers comprise an acetate buffer. In some embodiments, the one or more buffers comprise a combination of a phosphate buffer and an acetate buffer. In some embodiments, the one or more buffers comprise an acetate salt of an alkali metal. In some embodiments, the one or more buffers comprise a phosphate salt of an alkali metal. In some embodiments, the one or more buffers comprise sodium phosphate.

In some embodiments, formulation comprises one or more buffers at a concentration of between about 0.5 mM and between about 50 mM. In some embodiments, formulation comprises one or more buffers at a concentration of between about 1 mM and about 50 mM, between about 2.5 mM and about 50 mM, between about 5 mM and about 50 mM, between about 10 mM and about 50 mM, between about 15 mM and about 50 mM, between about 20 mM and about 50 mM, between about 25 mM and about 50 mM, between about 30 mM and about 50 mM, between about 35 mM and about 50 mM, between about 40 mM and about 50 mM, or between about 45 mM and about 50 mM. In some embodiments, formulation comprises one or more buffers at a concentration of between about 0.5 mM and about 45 mM, between about 0.5 mM and about 40 mM, between about 0.5 mM and about 35 mM, between about 0.5 mM and about 30 mM, between about 0.5 mM and 25 mM, between about 0.5 mM and about 20 mM, between about 0.5 mM and about 15 mM, between about 0.5 mM and about 10 mM, between about 0.5 mM and about 5 mM, between about 0.5 M and about 2.5 mM, or between 0.5 mM and about 1 mM. In some embodiments, formulation comprises one or more buffers at a concentration of between about 10 mM and about 30 mM. In some embodiments, formulation comprises one or more buffers at a concentration of between about 15 mM and about 25 mM. In some embodiments, formulation comprises one or more buffers at a concentration of about 20 mM.

In some embodiments, the formulation comprises sodium phosphate at a concentration of between about 0.5 mM and between about 50 mM. In some embodiments, the formulation comprises sodium phosphate at a concentration of between about 1 mM and about 50 mM, between about 2.5 mM and about 50 mM, between about 5 mM and about 50 mM, between about 10 mM and about 50 mM, between about 15 mM and about 50 mM, between about 20 mM and about 50 mM, between about 25 mM and about 50 mM, between about 30 mM and about 50 mM, between about 35 mM and about 50 mM, between about 40 mM and about 50 mM, or between about 45 mM and about 50 mM. In some embodiments, the formulation comprises sodium phosphate at a concentration of between about 0.5 mM and about 45 mM, between about 0.5 mM and about 40 mM, between about 0.5 mM and about 35 mM, between about 0.5 mM and about 30 mM, between about 0.5 mM and 25 mM, between about 0.5 mM and about 20 mM, between about 0.5 mM and about 15 mM, between about 0.5 mM and about 10 mM, between about 0.5 mM and about 5 mM, between about 0.5 M and about 2.5 mM, or between 0.5 mM and about 1 mM. In some embodiments, the formulation comprises sodium phosphate at a concentration of between about 10 mM and about 30 mM. In some embodiments, the formulation comprises sodium phosphate at a concentration of between about 15 mM and about 25 mM. In some embodiments, the formulation comprises sodium phosphate at a concentration of about 20 mM.

In some embodiments, the formulation further comprises an alkali metal salt. In some embodiments, the formulation further comprises a halide salt. In some embodiments, the formulation further comprises a halide salt of an alkali metal. In some embodiments, the formulation further comprises sodium chloride. In some embodiments, the formulation further comprises sodium chloride at a concentration of between about 25 mM and about 150 mM. In some embodiments, the formulation further comprises sodium chloride at a concentration of between about 30 mM and about 150 mM, between about 40 mM and about 150 mM, between about 50 mM and about 150 mM, between about 60 mM and about 150 mM, between about 70 mM and about 150 mM, between about 80 mM and about 150 mM, between about 90 mM and about 150 mM, between about 100 mM and about 150 mM, between about 110 mM and about 150 mM, between about 120 mM and about 150 mM, between about 130 mM and about 150 mM, or between about 140 mM and about 150 mM. In some embodiments, the formulation comprises sodium chloride at a concentration of between about 25 mM and about 140 mM, between about 25 mM and 130 mM, between about 25 mM and about 120 mM, between about 25 mM and about 110 mM, between about 25 mM and about 100 mM, between about 25 mM and about 90 mM, between about 25 mM and about 80 mM, between about 25 mM and about 70 mM, between about 25 mM and about 60 mM, between about 25 mM and about 50 mM, between about 25 mM and about 40 mM, or between about 25 mM and 30 mM. In some embodiments, the formulation comprises sodium chloride at a concentration of between about 30 mM and about 70 mM. In some embodiments, the formulation comprises sodium chloride at a concentration of between about 40 mM and about 60 mM. In some embodiments, the formulation comprises sodium chloride at a concentration of about 50 mM.

In some embodiments, the formulation further comprises one or more excipients. In some embodiments, the one or more excipients comprise one or more surfactants. In some embodiments, the one or more excipients comprise one or more emulsifiers. In some embodiments, the one or more excipients are derived from sorbitan. In some embodiments, the one or more excipients comprise polysorbate 80 (also known as PS-80). In some embodiments, the one or more excipients comprise polysorbate 20. In some embodiments, the one or more excipients comprise a combination of polysorbate 80 and polysorbate 20.

In some embodiments, the formulation comprises one or more excipients at a concentration of no more than about 0.3% (w/v). In some embodiments, the formulation comprises one or more excipients at a concentration of no more than about 0.28% (w/v). In some embodiments, the formulation comprises one or more excipients at a concentration of no more than about 0.26% (w/v). In some embodiments, the formulation comprises one or more excipients at a concentration of no more than about 0.24% (w/v). In some embodiments, the formulation comprises one or more excipients at a concentration of no more than about 0.22% (w/v). In some embodiments, the formulation further comprises one or more excipients at a concentration of between about 0.004% (w/v) and about 0.3% (w/v). In some embodiments, the formulation further comprises one or more excipients at a concentration of between about 0.01% (w/v) and about 0.3% (w/v).

In some embodiments, the formulation further comprises one or more excipients at a concentration of between about 0.004% (w/v) and about 0.2% (w/v). In some embodiments, the formulation further comprises one or more excipients at a concentration of between about 0.004% (w/v) and about 0.18% (w/v), between about 0.004% (w/v) and about 0.16% (w/v), between about 0.004% (w/v) and about 0.14% (w/v), between about 0.004% (w/v) and about 0.12% (w/v), between about 0.004% (w/v) and about 0.1% (w/v), between about 0.004% (w/v) and about 0.08% (w/v), between about 0.004% (w/v) and about 0.06% (w/v), between about 0.004% (w/v) and about 0.04% (w/v), between about 0.004% (w/v) and about 0.03%, between about 0.004% (w/v) and about 0.025% (w/v), between about 0.004% (w/v) and about 0.02% (w/v), between about 0.004% (w/v) and about 0.015% (w/v), or between about 0.004% (w/v) and about 0.01% (w/v). In some embodiments, the formulation further comprises one or more excipients at a concentration of between about 0.01% (w/v) and about 0.2% (w/v), between about 0.015% (w/v) and about 0.2% (w/v), between about 0.02% (w/v) and about 0.2% (w/v), between about 0.025% (w/v) and about 0.2% (w/v), between about 0.03% (w/v) and about 0.2% (w/v), between about 0.04% (w/v) and about 0.2% (w/v), between about 0.05% (w/v) and about 0.2% (w/v), between about 0.06% (w/v) and about 0.2% (w/v), between about 0.07% (w/v) and about 0.2% (w/v), between about 0.08% (w/v) and about 0.2% (w/v), between about 0.09% (w/v) and about 0.2% (w/v), between about 0.1% (w/v) and about 0.2% (w/v), between about 0.12% (w/v) and about 0.2% (w/v), between about 0.14% (w/v) and about 0.2% (w/v), between about 0.16% (w/v) and about 0.2% (w/v), or between about 0.18% (w/v) and 0.2% (w/v). In some embodiments, the formulation further comprises one or more excipients at a concentration of between about 0.01% (w/v) and about 0.03% (w/v). In some embodiments, the formulation further comprises one or more excipients at a concentration of between about 0.015% (w/v) and about 0.025% (w/v). In some embodiments, the formulation further comprises one or more excipients at a concentration of about 0.02% (w/v).

In some embodiments, the formulation further comprises polysorbate 80 at a concentration of between about 0.004% (w/v) and about 0.2% (w/v). In some embodiments, the formulation further comprises polysorbate 80 at a concentration of between about 0.004% (w/v) and about 0.18% (w/v), between about 0.004% (w/v) and about 0.16% (w/v), between about 0.004% (w/v) and about 0.14% (w/v), between about 0.004% (w/v) and about 0.12% (w/v), between about 0.004% (w/v) and about 0.1% (w/v), between about 0.004% (w/v) and about 0.08% (w/v), between about 0.004% (w/v) and about 0.06% (w/v), between about 0.004% (w/v) and about 0.04% (w/v), between about 0.004% (w/v) and about 0.03%, between about 0.004% (w/v) and about 0.025% (w/v), between about 0.004% (w/v) and about 0.02% (w/v), between about 0.004% (w/v) and about 0.015% (w/v), or between about 0.004% (w/v) and about 0.01% (w/v). In some embodiments, the formulation further comprises polysorbate 80 at a concentration of between about 0.01% (w/v) and about 0.2% (w/v), between about 0.015% (w/v) and about 0.2% (w/v), between about 0.02% (w/v) and about 0.2% (w/v), between about 0.025% (w/v) and about 0.2% (w/v), between about 0.03% (w/v) and about 0.2% (w/v), between about 0.04% (w/v) and about 0.2% (w/v), between about 0.05% (w/v) and about 0.2% (w/v), between about 0.06% (w/v) and about 0.2% (w/v), between about 0.07% (w/v) and about 0.2% (w/v), between about 0.08% (w/v) and about 0.2% (w/v), between about 0.09% (w/v) and about 0.2% (w/v), between about 0.1% (w/v) and about 0.2% (w/v), between about 0.12% (w/v) and about 0.2% (w/v), between about 0.14% (w/v) and about 0.2% (w/v), between about 0.16% (w/v) and about 0.2% (w/v), or between about 0.18% (w/v) and 0.2% (w/v). In some embodiments, the formulation further comprises polysorbate 80 at a concentration of between about 0.01% (w/v) and about 0.03% (w/v). In some embodiments, the formulation further comprises polysorbate 80 at a concentration of between about 0.015% (w/v) and about 0.025% (w/v). In some embodiments, the formulation further comprises polysorbate 80 at a concentration of about 0.02% (w/v).

In some embodiments, the formulation provided herein has a pH of between about 4.0 and about 8.5. In some embodiments, the formulation has a pH of between about 4.5 and about 8.5, between about 5.0 and about 8.5, between about 5.5 and about 8.5, between about 6.0 and about 8.5, between about 6.5 and about 8.5, between about 7.0 and about 8.5, between about 7.5 and about 8.5, or between about 8.0 and about 8.5. In some embodiments, the formulation has a pH of between about 4.5 and about 8.0, between about 4.5 and about 7.5, between about 4.5 and about 7.0, between about 4.5 and about 6.5, between about 4.5 and about 6.0, between about 4.5 and about 5.5, or between about 4.5 and about 5.0. In some embodiments, the formulation provided herein has a pH of between about 4.0 and about 8.0, between about 4.0 and about 7.5, between about 4.0 and about 7.0, between about 4.0 and about 6.5, between about 4.0 and about 6.0, between about 4.0 and about 5.5, between about 4.0 and about 5.0, or between about 4.0 and about 4.5. In some embodiments, the formulation provided herein has a pH of between about 5.5 and about 8.5. In some embodiments, the formulation provided herein has a pH of between about 6.0 and about 8.0. In some embodiments, the formulation provided herein has a pH of between 6.5 and about 7.5. In some embodiments, the formulation provided herein has a pH of around 7.0

In some embodiments, in conjunction with embodiments above or below, provided herein is an aqueous, non-lyophilized formulation, comprising: (i) an L-asparaginase, wherein the L-asparaginase comprises four monomer units, wherein each monomer unit has an amino acid sequence that is at least about 70% identical to SEQ ID NO: 1; and (ii) one or more stabilizers, or one or more buffers, or any combination thereof. In some embodiments, the L-asparaginase formulation comprises one or more stabilizers. In some embodiments, the one or more stabilizers comprise one or more disaccharides, one or more sorbitols, one or more amino acids, or any combination thereof. In some embodiments, the L-asparaginase formulation comprises one or more disaccharides. In some embodiments, the one or more disaccharides comprise trehalose, sucrose, or any combination thereof. In some embodiments, the L-sparaginase formulation comprises one or more buffers. In some embodiments, the one or more buffers, wherein the one or more buffers are substantially free of amino acid. In some embodiments, the L-asparaginase formulation comprises less than about 0.6% low-molecular-weight (LMW) species after storage at 40° C. for two months. In some embodiments, the L-asparaginase formulation comprises less than 2% high-molecular-weight (HMW) species after storage at 40° C. for two months. In some embodiments, the L-asparaginase formulation comprises less than about 0.6% low-molecular-weight (LMW) species and less than 2% high-molecular-weight (HMW) species after storage at 40° ° C. for two months. In some embodiments, the L-asparaginase formulation comprises less than about 0.6% low-molecular-weight (LMW) species after storage at 37° ° C. for one week. In some embodiments, the formulation has a pH of between about 4.0 and about 8.5. In some embodiments, the formulation has a pH of about 7.0.

In some embodiments, in conjunction with embodiments above or below, (i) the L-asparaginase comprises four monomer units, wherein each monomer unit has an amino acid sequence that is at least about 70% identical to SEQ ID NO: 1, and is present at a concentration of around 20 mg/mL; (ii) one or more stabilizers (e.g., disaccharides, sorbitols, amino acids, or any combination thereof) at a concentration between about 50 mM and about 300 mM; (iii) one or more buffers (e.g., acetate, glutamate, citrate, histidine, succinate, phosphate, hydroxymethylaminomethane, or combination thereof) at a concentration of between about 0.5 mM and about 50 mM; (iv) sodium chloride, wherein the sodium chloride is present at a concentration of between about 25 mM and about 150 mM; and (v) one or more surfactants (e.g., polysorbate 80, polysorbate 20, poloxamer 188, or any combination thereof) at a concentration of between about 0.004% (w/v) and about 0.3% (w/v). In some embodiments, in conjunction with embodiments above or below, the formulation has a pH of between about 4.0 and about 8.5. In some embodiments, in conjunction with embodiments above or below, the formulation has a pH of about 7.0.

In some embodiments, in conjunction with embodiments above or below, (i) the L-asparaginase comprises four monomer units, wherein each monomer unit has an amino acid sequence that is at least about 70% identical to SEQ ID NO: 1, and is present at a concentration of around 20 mg/mL; (ii) trehalose, wherein the trehalose is present at a concentration between about 50 mM and about 300 mM; (iii) sodium phosphate, wherein the sodium phosphate (e.g., dibasic anhydrous, monobasic monohydrate, or combination thereof) is present at a concentration of between about 0.5 mM and about 50 mM; (iv) sodium chloride, wherein the sodium chloride is present at a concentration of between about 25 mM and about 150 mM; and (v) polysorbate 80, wherein the polysorbate 80 is present at a concentration of between about 0.004% (w/v) and about 0.2% (w/v).

In some embodiments, in conjunctions with embodiments above or below, the L-asparaginase can be formulated in a unit dosage form. The term “unit dosage forms” refers to physically discrete units suitable as unitary dosages for human subjects and other mammals, each unit containing a predetermined quantity of active ingredient calculated to produce the desired therapeutic effect, in association with a suitable pharmaceutical excipient. In some embodiments, a unit dosage can be administered every 12 hours, every 24 hours, every 48 hours, or every 72 hours. In some embodiments, a unit dosage is administered every 48 hours. In some embodiments, a unit dosage contain from about 5 mg to about 50 mg (e.g., about 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, and 49 mg) of the L-asparaginase.

In some embodiments, provided herein is an aqueous, non-lyophilized formulation, comprising: (i) an L-asparaginase, wherein the L-asparaginase comprises four monomer units, wherein each monomer unit has an amino acid sequence that is at least about 95% identical to SEQ ID NO: 1; (ii) one or more disaccharides, wherein the one or more disaccharides comprise trehalose, sucrose, or any combination thereof; (iii) one or more buffers, wherein the one or more buffers are substantially free of amino acid; and (iv) sodium chloride, wherein the formulation comprises less than about 5% low-molecular-weight (LMW) species after storage at 37° C. for one week. In some embodiments, the formulation has a pH of between about 4.0 and about 8.5. In some embodiments, the formulation has a pH of about 7.0.

In some embodiments, provided herein is an aqueous, non-lyophilized formulation, comprising: (i) an L-asparaginase, wherein the L-asparaginase comprises four monomer units, wherein each monomer unit has an amino acid sequence that is at least about 95% identical to SEQ ID NO: 1; (ii) one or more disaccharides, wherein the one or more disaccharides comprise trehalose, sucrose, or any combination thereof; (iii) one or more buffers, wherein the one or more buffers are substantially free of amino acid; and (iv) one or more excipients, wherein the formulation comprises less than about 5% low-molecular-weight (LMW) species after storage at 37° ° C. for one week.

In some embodiments, provided herein is an aqueous, non-lyophilized formulation, comprising: (i) an L-asparaginase, wherein the L-asparaginase comprises four monomer units, wherein each monomer unit has an amino acid sequence that is at least about 95% identical to SEQ ID NO: 1; (ii) one or more disaccharides, wherein the one or more disaccharides comprise trehalose, sucrose, or any combination thereof; (iii) one or more buffers, wherein the one or more buffers are substantially free of amino acid; (iv) sodium chloride; and (v) one or more excipients, wherein the formulation comprises less than about 5% low-molecular-weight (LMW) species after storage at 37° C. for one week. In some embodiments, the formulation has a pH of between about 4.0 and about 8.5. In some embodiments, the formulation has a pH of about 7.0.

In some embodiments, provided herein is an aqueous, non-lyophilized formulation, comprising: (i) an L-asparaginase, wherein the L-asparaginase comprises four monomer units, wherein each monomer unit has an amino acid sequence that is at least about 95% identical to SEQ ID NO: 1; (ii) one or more disaccharides, wherein the one or more disaccharides comprise trehalose, sucrose, or any combination thereof; (iii) one or more buffers, wherein the one or more buffers are substantially free of amino acid; and (iv) polysorbate 80, wherein the formulation comprises less than about 5% low-molecular-weight (LMW) species after storage at 37° C. for one week. In some embodiments, the formulation has a pH of between about 4.0 and about 8.5. In some embodiments, the formulation has a pH of about 7.0.

In some embodiments, provided herein is an aqueous, non-lyophilized formulation, comprising: (i) an L-asparaginase, wherein the L-asparaginase comprises four monomer units, wherein each monomer unit has an amino acid sequence that is at least about 95% identical to SEQ ID NO: 1; (ii) one or more disaccharides, wherein the one or more disaccharides comprise trehalose, sucrose, or any combination thereof; (iii) one or more buffers, wherein the one or more buffers are substantially free of amino acid; (iv) sodium chloride; and (v) polysorbate 80, wherein the formulation comprises less than about 5% low-molecular-weight (LMW) species after storage at 37° C. for one week. In some embodiments, the formulation has a pH of between about 4.0 and about 8.5. In some embodiments, the formulation has a pH of about 7.0.

In some embodiments, provided herein is an aqueous, non-lyophilized formulation, comprising: (i) an L-asparaginase, wherein the L-asparaginase comprises four monomer units, wherein each monomer unit has an amino acid sequence that is at least about 95% identical to SEQ ID NO: 1; (ii) trehalose; (iii) sodium phosphate; (iv) sodium chloride; and (v) polysorbate 80, wherein the formulation comprises less than about 5% low-molecular-weight (LMW) species after storage at 37° ° C. for one week. In some embodiments, the formulation has a pH of between about 4.0 and about 8.5. In some embodiments, the formulation has a pH of about 7.0.

In some embodiments, provided herein is an aqueous, non-lyophilized formulation, comprising: (i) an L-asparaginase, wherein the L-asparaginase comprises four monomer units, wherein each monomer unit has an amino acid sequence that is at least about 95% identical to SEQ ID NO. 1, and wherein the L-asparaginase is present at a concentration of around 20 mg/mL; (ii) trehalose, wherein the trehalose is present at a concentration between about 50 mM and about 300 mM; (iii) sodium phosphate, wherein the sodium phosphate is present at a concentration of between about 0.5 mM and about 50 mM; (iv) sodium chloride, wherein the sodium chloride is present at a concentration of between about 25 mM and about 150 mM; and (v) polysorbate 80, wherein the polysorbate 80 is present at a concentration of between about 0.004% (w/v) and about 0.2% (w/v), wherein the formulation comprises less than about 5% low-molecular-weight (LMW) species after storage at 37° ° C. for one week. In some embodiments, the formulation has a pH of between about 4.0 and about 8.5. In some embodiments, the formulation has a pH of about 7.0.

In some embodiments, provided herein is an aqueous, non-lyophilized formulation, comprising: (i) an L-asparaginase, wherein the L-asparaginase comprises four monomer units, wherein each monomer unit has an amino acid sequence that is at least about 95% identical to SEQ ID NO: 1, and wherein the L-asparaginase is present at a concentration of around 20 mg/mL; (ii) trehalose, wherein the trehalose is present at a concentration of about 170 mM; (iii) sodium phosphate, wherein the sodium phosphate is present at a concentration of about 20 mM; (iv) sodium chloride, wherein the sodium chloride is present at a concentration of about 50 mM; and (v) polysorbate 80, wherein the polysorbate 80 is present at a concentration of about 0.02% (w/v), wherein the formulation comprises less than about 5% low-molecular-weight (LMW) species after storage at 37° ° C. for one week. In some embodiments, the formulation has a pH of between about 4.0 and about 8.5. In some embodiments, the formulation has a pH of about 7.0.

In some embodiments, provided herein is a formulation as described elsewhere herein, wherein the formulation comprises less than about 5% low-molecular-weight (LMW) species after storage at 37° C. for one week. In some embodiments, the formulation comprises less than about 4.5% low-molecular-weight (LMW) species after storage at 37° C. for one week. In some embodiments, the formulation comprises less than about 4% low-molecular-weight (LMW) species after storage at 37° C. for one week. In some embodiments, the formulation comprises less than about 3.5% low-molecular-weight (LMW) species after storage at 37° C. for one week. In some embodiments, the formulation comprises less than about 3% low-molecular-weight (LMW) species after storage at 37° C. for one week. In some embodiments, the formulation comprises less than about 2.5% low-molecular-weight (LMW) species after storage at 37° ° C. for one week. In some embodiments, the formulation comprises less than about 2% low-molecular-weight (LMW) species after storage at 37° C. for one week. the formulation comprises less than about 1.5% low-molecular-weight (LMW) species after storage at 37° C. for one week. In some embodiments, the formulation comprises less than about 1% low-molecular-weight (LMW) species after storage at 37° C. for one week. In some embodiments, the formulation comprises less than about 0.5% low-molecular-weight (LMW) species after storage at 37° ° C. for one week.

It is to be understood that each component and/or feature of the formulations described herein may be present in combination with any other component and/or feature of the formulations described herein, the same as if each and every combination of the components and/or features described herein were individually and specifically listed.

### Methods of Treatment

The L-asparaginase formulations of the present disclosure can be used in the treatment of a disease in a subject (for example, a human), wherein the disease is treatable by the depletion of asparagine or the administration of asparaginase. In some embodiments, the human subject has, prior to administration of the L-asparaginase, experienced silent inactivation of the E. Coli-derived asparaginase. In some embodiments, the subject has, prior to administration of the L-asparaginase, experienced an allergic reaction to the E. Coli-derived asparaginase. In some embodiments, the subject has, prior to administration of the L-asparaginase, experienced anaphylaxis to the E. Coli-derived asparaginase. Non-limiting examples of objective signs of allergy or hypersensitivity include testing “antibody positive” for an asparaginase enzyme.

In some embodiments, the L-asparaginase formulations of the present disclosure are useful in the treatment or the manufacture of a medicament for use in the treatment of acute lymphoblastic leukemia (ALL). The incidence of relapse in ALL patients following treatment with L-asparaginase remains high, with approximately 10-25% of pediatric ALL patients having early relapse (e.g., some during maintenance phase at 30-36 months post-induction). If a patient treated with E. coli-derived L-asparaginase has a relapse, subsequent treatment with E. coli preparations could lead to a “vaccination” effect, whereby the E. coli preparation has increased immunogenicity during the subsequent administrations. In one embodiment, the L-asparaginase of the invention may be used in a method of treating patients with relapsed ALL who were previously treated with other asparaginase preparations, in particular those who were previously treated with E. coli-derived asparaginases.

Diseases or disorders that the L-asparaginase formulations of the present disclosure are useful in treating include, but are not limited to, the following: malignancies, or cancers, including but not limited to, hematalogic malignancies, lymphoma, non-Hodgkin's lymphoma, NK lymphoma, pancreatic cancer, Hodgkin's disease, large cell immunoblastic lymphoma, acute promyelocytic leukemia, acute myelocytic leukemia, acute myelomonocytic leukemia, acute monocytic leukemia, acute T-cell leukemia, acute myeloid leukemia (AML), biphenotypic B-cell myelomonocytic Leukemia, chronic lymphocytic leukemia, lymphosarcoma, reticulosarcoma, and melanosarcoma, and diffuse large B-cell lymphoma (DLBCL). Other diseases or disorders that the L-asparaginase formulations useful in treating are cancers including, but not limited to, renal cell carcinoma, renal cell adenocarcinoma, glioblastoma including glioblastoma multiforma and glioblastoma astrocytoma, medulloblastoma, rhabdomyosarcoma, malignant melanoma, epidermoid carcinoma, squamous cell carcinoma, lung carcinoma including large cell lung carcinoma and small cell lung carcinoma, endometrial carcinoma, ovarian adenocarcinoma, ovarian tetratocarcinoma, cervical adenocarcinoma, breast carcinoma, breast adenocarcinoma, breast ductal carcinoma, pancreatic adenocarcinoma, pancreatic ductal carcinoma, colon carcinoma, colon adenocarcinoma, colorectal adenocarcinoma, bladder transitional cell carcinoma, bladder papilloma, prostate carcinoma, osteosarcoma, epitheloid carcinoma of the bone, prostate carcinoma, and thyroid cancer. The cancer may be a solid cancer, for example lung cancer or breast cancer. Representative non-malignant hematologic diseases which respond to asparagine depletion include immune system-mediated blood diseases, including, but not limited to, infectious diseases such as those caused by HIV infection (i.e., AIDS). Nonhematologic diseases associated with asparagine dependence include autoimmune diseases, for example, rheumatoid arthritis, collagen vascular diseases, AIDS, osteoarthritis, Issac's syndrome, psoriasis, insulin dependent diabetes mellitus, multiple sclerosis, sclerosing panencephalitis, systemic lupus erythematosus (SLE), rheumatic fever, inflammatory bowel disease (e.g., ulcerative colitis and Crohn's disease), primary billiary cirrhosis, chronic active hepatitis, glomerulonephritis, myasthenia gravis, pemphigus vulgaris, and Graves' disease. Cells suspected of causing disease can be tested for asparagine dependence in any suitable in vitro or in vivo assay, e.g., an in vitro assay wherein the growth medium lacks asparagine.

In some embodiments, in conjunction with embodiments above or below, the disease, condition, or disorder that is treatable by asparagine depletion in a subject in need thereof comprises WNT mutated colorectal cancer (CRC). In some embodiments, in conjunction with embodiments above or below, the disease, condition, or disorder that is treatable by asparagine depletion in a subject in need thereof comprises relapse remitting (R/R) acute myeloid leukemia (AML).

Diseases or disorders that the L-asparaginase formulations of the present disclosure are useful in treating include, but are not limited to, sarcoma, breast cancer, metastatic breast cancer, liver cancer, stomach cancer, colorectal cancer, and head and neck cancer.

### Pharmaceutical Compositions and Co-Administration

In some embodiments, treatment with a L-asparaginase formulation of the present disclosure is co-administered with a multi-agent chemotherapeutic regimen. In some embodiments, treatment with a L-asparaginase formulation of the present disclosure is co-administered with one or more other chemotherapeutic agents as part of a multi-agent chemotherapeutic regimen. In some embodiments, treating patients with a L-asparaginase formulation of the present disclosure in addition to other agents helps to ensure availability of an asparaginase for patients who have developed hypersensitivity to E. coli derivedasparaginase.

Examples of agents that may be part of a multi-agent chemotherapeutic regimen with a L-asparaginase of the present disclosure include, but are not limited to: cytarabine, vincristine, daunorubicin, methotrexate, leuvocorin, doxorubicin, anthracycline, corticosteroids and glucocortiods (including but not limited to prednisone, prednisolone, and/or dexamethasone), cyclophosphamide, 6-mercaptopurine, venetoclax, and etoposide. In some embodiments, the multi-agent chemotherapeutic regimen is the L-asparaginase and one additional chemotherapeutic agent. In some embodiments, the multi-agent chemotherapeutic regimen is the L-asparaginase and two or more additional chemotherapeutic agents. As an example, patients with ALL will be co-administered the L-asparaginase of the present disclosure along with a multi-agent chemotherapy during 3 chemotherapy phases including induction, consolidation or intensification, and maintenance. In a specific example, the L-asparaginase formulation of the present disclosure is co-administered with an asparagine synthetase inhibitor (e.g., such as set forth in WO 2007/103290, which is herein incorporated by reference in its entirety). In another specific example, the L-asparaginase formulation of the present disclosure is not co-administered with an asparagine synthetase inhibitor, but is co-administered with other chemotherapy drugs. In another specific example, the L-asparaginase formulation of the present disclosure is co-administered with an asparagine synthetase inhibitor and other chemotherapy drugs. The L-asparaginase formulation of the present disclosure can be co-administered before, after, or simultaneously with other compounds as part of a multi-agent chemotherapy regimen. In a particular embodiment, the L-asparaginase of the present disclosure comprises a protein recombinantly produced in Pseudomonas fluorescens and, more specifically, the L-asparaginase comprising the sequence of SEQ ID NO: 1.

The formulations described herein can be administered to a patient as a pharmaceutical composition using standard techniques. Techniques and formulations generally may be found in Remington's Pharmaceutical Sciences, 22nd edition, Mack Publishing, 2015 (herein incorporated by reference).

Suitable dosage forms, in part, depend upon the use or the route of entry, for example, oral, transdermal, transmucosal, or by injection (parenteral). Such dosage forms should allow the therapeutic agent to reach a target cell or otherwise have the desired therapeutic effect. For example, pharmaceutical compositions injected into the blood stream preferably are soluble. The pharmaceutical compositions according to the present disclosure can be formulated as pharmaceutically acceptable salts and complexes thereof. Pharmaceutically acceptable salts are non-toxic salts present in the amounts and concentrations at which they are administered. The preparation of such salts can facilitate pharmaceutical use by altering the physical characteristics of the compound without preventing it from exerting its physiological effect. Useful alterations in physical properties include lowering the melting point to facilitate transmucosal administration and increasing solubility to facilitate administering higher concentrations of the drug. The pharmaceutically acceptable salt of a modified protein as described herein may be present as a complex, as those in the art will appreciate. Pharmaceutically acceptable salts include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate, and quinate. Pharmaceutically acceptable salts can be obtained from acids, including hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid. Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, supra. Such salts can be prepared using the appropriate corresponding bases. Pharmaceutically acceptable carriers and/or excipients can also be incorporated into a pharmaceutical composition according to the invention to facilitate administration of the particular asparaginase. Examples of carriers suitable for use in the practice of the invention include calcium carbonate, calcium phosphate, various sugars such as lactose, glucose, or sucrose, or types of starch, cellulose derivatives, gelatin, vegetable oils, polyethylene glycols, and physiologically compatible solvents. Examples of physiologically compatible solvents include sterile solutions of water for injection (WFI), saline solution and dextrose.

Pharmaceutical compositions according to the invention can be administered by different routes, including intravenous, intraperitoneal, subcutaneous, intramuscular, oral, topical (transdermal), or transmucosal administration. For oral administration, for example, the compounds can be formulated into conventional oral dosage forms such as capsules, tablets, and liquid preparations such as syrups, elixirs, and concentrated drops. Alternatively, injection (parenteral administration) may be used, e.g., intramuscular, intravenous, intraperitoneal, and subcutaneous injection. For injection, pharmaceutical compositions are formulated in liquid solutions, preferably in physiologically compatible buffers or solutions, such as saline solution, Hank's solution, or Ringer's solution. In a specific aspect, the L-asparaginase formulation is administered intramuscularly. In a specific aspect, the L-asparaginase formulation is administered intravenously. In a specific aspect, the L-asparaginase formulation is administered subcutaneously.

Systemic administration can also be accomplished by transmucosal or transdermal means. For transmucosal or transdermal administration, penetrants appropriate to the barrier to be permeated are used in the formulation. Such penetrants are well known in the art, and include, for example, for transmucosal administration, bile salts, and fusidic acid derivatives. In addition, detergents may be used to facilitate permeation. Transmucosal administration, for example, may be through nasal sprays, inhalers (for pulmonary delivery), rectal suppositories, or vaginal suppositories. For topical administration, compounds can be formulated into ointments, salves, gels, or creams, as is well known in the art.

### Dosing

In some embodiments, a dose is an amount administered to the human subject over a certain time and frequency. In some embodiments, the dose of L-asparaginase formulation will be given to a human subject with hypersensitivity only when the hypersensitivity subsides.

In an exemplary embodiment, a L-asparaginase formulation is administered to a human subject in an amount from about 10 mg/m² to 100 mg/m². In an exemplary embodiment, a L-asparaginase formulation is administered intramuscularly every other day over a period of 5 consecutive days followed by a rest period of 2 consecutive days, wherein the amount is about 25 mg/m². In an exemplary embodiment, a L-asparaginase formulation is administered intravenously every other day over a period of 5 consecutive days followed by a rest period of 2 consecutive days, wherein the amount is about 37.5 mg/m². In an exemplary embodiment, a L-asparaginase formulation is administered intravenously every other day over a period of 5 consecutive days followed by a rest period of 2 consecutive days, wherein the amount is about 50 mg/m².

In further exemplary embodiments and in accordance with any of the discussion herein regarding dosing, the L-asparaginase formulation administered in such doses is not conjugated to a polymer such as a PEG moiety and/or is not conjugated to a peptide comprising solely alanine and/or proline residues.

### 1. Dose Amount

The amounts of the L-asparaginase formulation of the present disclosure that are to be delivered will depend on many factors, for example, the IC50, EC50, the biological half-life of the compound, the age, size, weight, and physical condition of the patient, and the disease or disorder to be treated. The importance of these and other factors to be considered are well known to those of ordinary skill in the art. Generally, the amount of the L-asparaginase formulation of the present disclosure will be administered at a range from about 1 milligram per square meter of the surface area of the patient's body (mg/m²) to 1,000/m², with a dosage range of about 10 mg/m² to about 100 mg/m² to treat disease, including but not limited to ALL or LBL. Of course, other dosages and/or treatment regimens may be employed, as determined by the attending physician.

In some embodiments, the method comprises administering the recombinant L-asparaginase of the present disclosure at an amount from about 10 mg/m² and about 100 mg/m². In some embodiments, the method comprises administering the L-asparaginase formulation of the present disclosure at an amount from 10 mg/m² and 100 mg/m². In some embodiments, the L-asparaginase formulation of the present disclosure is administered in an amount of about 10, 15, 20, 25, 30, 35, 40, 45, 50, 55, 60, 65, 70, 75, 80, 85, 95, or 95 mg/m² or an equivalent amount thereof (for example on a protein content basis). In a more specific embodiment, the L-asparaginase formulation of the present disclosure is administered at an amount selected from the group consisting of about 10, 20, 30, 40, 50, 60, 70, 80, 90, and about 100 mg/m². In another specific embodiment, the L-asparaginase formulation of the present disclosure is administered at a dose more than or equal to about 1, 10, 15, 20, 25, 30, 35, 40, 45, 50, 55, 60, 65, 70, 75, 80, 85, 95, 100, 200, or 300 mg/m². In another specific embodiment, the L-asparaginase formulation of the present disclosure is administered at a dose less than or equal to about 300, 200 100, 95, 80, 75, 70, 65, 60, 55, 50, 45, 40, 35, 30, 25, 20, 15, 10, 5, or 1 mg/m². In an exemplary embodiment, the L-asparaginase formulation of the present disclosure is administered in an amount between about 12 mg/m² and about 90 mg/m². In another exemplary embodiment, the recombinant L-asparaginase of the present disclosure is administered in an amount between about 20 mg/m² and about 80 mg/m². In another exemplary embodiment, the L-asparaginase formulation of the present disclosure is administered in an amount between about 25 mg/m² and about 70 mg/m². In an exemplary embodiment, the L-asparaginase formulation of the present disclosure is administered in an amount between about 25 mg/m² and about 80 mg/m². In an exemplary embodiment, the L-asparaginase formulation of the present disclosure is administered in an amount between about 37.5 mg/m² and about 80 mg/m². In an exemplary embodiment, the L-asparaginase formulation of the present disclosure is administered in an amount between about 37.5 mg/m² and about 65 mg/m². In an exemplary embodiment, the recombinant L-asparaginase of the present disclosure is administered in an amount between about 25 mg/m² and about 37.5 mg/m². In an exemplary embodiment, the L-asparaginase formulation of the present disclosure is administered in an amount between about 25 mg/m² and about 100 mg/m². In an exemplary embodiment, the L-asparaginase formulation of the present disclosure is administered in an amount between about 25 mg/m² and about 65 mg/m². In an exemplary embodiment, the L-asparaginase formulation of the present disclosure is administered in an amount between about 25 mg/m² and about 80 mg/m².

In some embodiments, the method comprises administering the recombinant L-asparaginase of the present disclosure at an amount from about 25 mg/m² and about 50 mg/m². In some embodiments, the method comprises administering the L-asparaginase formulation of the present disclosure at an amount from 25 mg/m² and 50 mg/m². In some embodiments, the L-asparaginase formulation of the present disclosure is administered in an amount of about 25, 26, 27, 28, 29, 30, 31, 32, 33, 34, 35, 36, 37, 38, 39, 40, 41, 42, 43, 44, 45, 46, 47, 48, 49, or 50 mg/m² or an equivalent amount thereof (for example on a protein content basis).

In an exemplary embodiment, the L-asparaginase formulation of the present disclosure is administered in an amount between about 20 mg/m² and about 30 mg/m². In an exemplary embodiment, the L-asparaginase formulation of the present disclosure is administered in an amount between about 22.5 mg/m² and about 28.5 mg/m². In an exemplary embodiment, the L-asparaginase formulation of the present disclosure is administered in an amount between about 23 mg/m² and about 27 mg/m². In an exemplary embodiment, the recombinant L-asparaginase of the present disclosure is administered in an amount between about 24 mg/m² and about 26 mg/m². In an exemplary embodiment, the L-asparaginase formulation of the present disclosure is administered in an amount between about 24.5 mg/m² and about 25.5 mg/m².

In an exemplary embodiment, the L-asparaginase formulation of the present disclosure is administered in an amount between about 40 mg/m² and about 60 mg/m². In an exemplary embodiment, the L-asparaginase formulation of the present disclosure is administered in an amount between about 42.5 mg/m² and about 58.5 mg/m². In an exemplary embodiment, the L-asparaginase formulation of the present disclosure is administered in an amount between about 43 mg/m² and about 57 mg/m². In an exemplary embodiment, the recombinant L-asparaginase of the present disclosure is administered in an amount between about 44 mg/m² and about 56 mg/m². In an exemplary embodiment, the L-asparaginase formulation of the present disclosure is administered in an amount between about 45 mg/m² and about 55 mg/m². In an exemplary embodiment, the L-asparaginase formulation of the present disclosure is administered in an amount between about 46 mg/m² and about 54 mg/m². In an exemplary embodiment, the L-asparaginase formulation of the present disclosure is administered in an amount between about 47.5 mg/m² and about 52.5 mg/m². In an exemplary embodiment, the L-asparaginase formulation of the present disclosure is administered in an amount between about 48 mg/m² and about 52 mg/m². In an exemplary embodiment, the recombinant L-asparaginase of the present disclosure is administered in an amount between about 49 mg/m² and about 51 mg/m². In an exemplary embodiment, the L-asparaginase formulation of the present disclosure is administered in an amount between about 49.5 mg/m² and about 50.5 mg/m².

In an exemplary embodiment, the L-asparaginase formulation of the present disclosure is administered in an amount between about 30 mg/m² and about 75 mg/m². In an exemplary embodiment, the L-asparaginase formulation of the present disclosure is administered in an amount between about 35 mg/m² and about 70 mg/m². In an exemplary embodiment, the L-asparaginase formulation of the present disclosure is administered in an amount between about 40 mg/m² and about 65 mg/m². In an exemplary embodiment, the recombinant L-asparaginase of the present disclosure is administered in an amount between about 45 mg/m² and about 60 mg/m². In an exemplary embodiment, the L-asparaginase formulation of the present disclosure is administered in an amount between about 50 mg/m² and about 55 mg/m².

In an exemplary embodiment, the L-asparaginase formulation of the present disclosure is administered in an amount between about 40 mg/m² and about 75 mg/m². In an exemplary embodiment, the L-asparaginase formulation of the present disclosure is administered in an amount between about 45 mg/m² and about 70 mg/m². In an exemplary embodiment, the L-asparaginase formulation of the present disclosure is administered in an amount between about 50 mg/m² and about 65 mg/m². In an exemplary embodiment, the recombinant L-asparaginase of the present disclosure is administered in an amount between about 55 mg/m² and about 60 mg/m².

In an exemplary embodiment, the L-asparaginase formulation of the present disclosure is administered in an amount between about 40 mg/m² and about 60 mg/m². In an exemplary embodiment, the L-asparaginase formulation of the present disclosure is administered in an amount between about 45 mg/m² and about 55 mg/m². In an exemplary embodiment, the L-asparaginase formulation of the present disclosure is administered in an amount between about 47.5 mg/m² and about 50 mg/m².

In an exemplary embodiment, the L-asparaginase formulation of the present disclosure is administered in an amount between about 30 mg/m² and about 35 mg/m².

In an exemplary embodiment, the L-asparaginase formulation of the present disclosure is administered in an amount between about 30 mg/m² and about 95 mg/m². In an exemplary embodiment, the L-asparaginase formulation of the present disclosure is administered in an amount between about 35 mg/m² and about 90 mg/m². In an exemplary embodiment, the L-asparaginase formulation of the present disclosure is administered in an amount between about 40 mg/m² and about 85 mg/m². In an exemplary embodiment, the L-asparaginase formulation of the present disclosure is administered in an amount between about 45 mg/m² and about 80 mg/m². In an exemplary embodiment, the L-asparaginase formulation of the present disclosure is administered in an amount between about 50 mg/m² and about 75 mg/m². In an exemplary embodiment, the L-asparaginase formulation of the present disclosure is administered in an amount between about 55 mg/m² and about 70 mg/m². In an exemplary embodiment, the L-asparaginase formulation of the present disclosure is administered in an amount between about 60 mg/m² and about 65 mg/m².

In an exemplary embodiment, the L-asparaginase formulation of the present disclosure is administered in an amount between about 30 mg/m² and about 60 mg/m². In an exemplary embodiment, the L-asparaginase formulation of the present disclosure is administered in an amount between about 35 mg/m² and about 55 mg/m². In an exemplary embodiment, the L-asparaginase formulation of the present disclosure is administered in an amount between about 40 mg/m² and about 50 mg/m². In an exemplary embodiment, the L-asparaginase formulation of the present disclosure is administered in an amount between about 42.5 mg/m² and about 57.5 mg/m².

In an exemplary embodiment, the L-asparaginase formulation of the present disclosure is administered in an amount between about 30 mg/m² and about 75 mg/m². In an exemplary embodiment, the L-asparaginase formulation of the present disclosure is administered in an amount between about 35 mg/m² and about 70 mg/m². In an exemplary embodiment, the L-asparaginase formulation of the present disclosure is administered in an amount between about 40 mg/m² and about 65 mg/m². In an exemplary embodiment, the L-asparaginase formulation of the present disclosure is administered in an amount between about 45 mg/m² and about 60 mg/m². In an exemplary embodiment, the L-asparaginase formulation of the present disclosure is administered in an amount between about 50 mg/m² and about 55 mg/m².

In some embodiments, the L-asparaginase formulation of the present disclosure is administered intramuscularly in an amount of between about 10 mg/m² and about 50 mg/m². In some embodiments, the L-asparaginase formulation of the present disclosure is administered intramuscularly in an amount of between about 12.5 mg/m² and about 47.5 mg/m². In some embodiments, the L-asparaginase formulation of the present disclosure is administered intramuscularly in an amount of between about 15 mg/m² and about 45 mg/m². In some embodiments, the L-asparaginase formulation of the present disclosure is administered intramuscularly in an amount of between about 20 mg/m² and about 42.5 mg/m². In some embodiments, the L-asparaginase formulation of the present disclosure is administered intramuscularly in an amount of between about 22.5 mg/m² and about 40 mg/m². In some embodiments, the L-asparaginase formulation of the present disclosure is administered intramuscularly in an amount of between about 24 mg/m² and about 39 mg/m². In some embodiments, the L-asparaginase formulation of the present disclosure is administered intramuscularly in an amount of between about 27 mg/m² and about 37.5 mg/m². In some embodiments, the L-asparaginase formulation of the present disclosure is administered intramuscularly in an amount of between about 30 mg/m² and about 45 mg/m². In some embodiments, the L-asparaginase formulation of the present disclosure is administered intramuscularly in an amount of about 25 mg/m². In some embodiments, the recombinant L-asparaginase of the present disclosure is administered intramuscularly in an amount of 25 mg/m². In an exemplary embodiment, the L-asparaginase formulation of the present disclosure is administered intramuscularly in an amount between about 25 mg/m² and about 80 mg/m². In an exemplary embodiment, the L-asparaginase formulation of the present disclosure is administered intramuscularly in an amount between about 37.5 mg/m² and about 80 mg/m². In an exemplary embodiment, the L-asparaginase formulation of the present disclosure is administered intramuscularly in an amount between about 37.5 mg/m² and about 65 mg/m². In an exemplary embodiment, the L-asparaginase formulation of the present disclosure is administered intramuscularly in an amount between about 25 mg/m² and about 37.5 mg/m². In an exemplary embodiment, the L-asparaginase formulation of the present disclosure is administered intramuscularly in an amount between about 30 mg/m² and about 75 mg/m². In an exemplary embodiment, the L-asparaginase formulation of the present disclosure is administered intramuscularly in an amount between about 35 mg/m² and about 70 mg/m². In an exemplary embodiment, the L-asparaginase formulation of the present disclosure is administered intramuscularly in an amount between about 40 mg/m² and about 65 mg/m². In an exemplary embodiment, the L-asparaginase formulation of the present disclosure is administered intramuscularly in an amount between about 45 mg/m² and about 60 mg/m². In an exemplary embodiment, the L-asparaginase formulation of the present disclosure is administered intramuscularly in an amount between about 50 mg/m² and about 55 mg/m². In an exemplary embodiment, the L-asparaginase formulation of the present disclosure is administered intramuscularly in an amount between about 40 mg/m² and about 75 mg/m². In an exemplary embodiment, the L-asparaginase formulation of the present disclosure is administered intramuscularly in an amount between about 45 mg/m² and about 70 mg/m². In an exemplary embodiment, the L-asparaginase formulation of the present disclosure is administered intramuscularly in an amount between about 50 mg/m² and about 65 mg/m². In an exemplary embodiment, the L-asparaginase formulation of the present disclosure is administered intramuscularly in an amount between about 55 mg/m² and about 60 mg/m². In an exemplary embodiment, the L-asparaginase formulation of the present disclosure is administered intramuscularly in an amount between about 40 mg/m² and about 60 mg/m². In an exemplary embodiment, the L-asparaginase formulation of the present disclosure is administered intramuscularly in an amount between about 45 mg/m² and about 55 mg/m². In an exemplary embodiment, the L-asparaginase formulation of the present disclosure is administered intramuscularly in an amount between about 47.5 mg/m² and about 50 mg/m². In an exemplary embodiment, the L-asparaginase formulation of the present disclosure is administered intramuscularly in an amount between about 30 mg/m² and about 35 mg/m².

In some embodiments, the L-asparaginase formulation of the present disclosure is administered intravenously in an amount of between about 10 mg/m² and about 95 mg/m². In some embodiments, the L-asparaginase formulation of the present disclosure is administered intravenously in an amount of between about 20 mg/m² and about 60 mg/m². In some embodiments, the L-asparaginase formulation of the present disclosure is administered intravenously in an amount of between about 22.5 mg/m² and about 57.5 mg/m². In some embodiments, the L-asparaginase formulation of the present disclosure is administered intravenously in an amount of between about 25 mg/m² and about 55 mg/m². In some embodiments, the L-asparaginase formulation of the present disclosure is administered intravenously in an amount of between about 27.5 mg/m² and about 47.5 mg/m². In some embodiments, the L-asparaginase formulation of the present disclosure is administered intravenously in an amount of between about 30 mg/m² and about 45 mg/m². In some embodiments, the L-asparaginase formulation of the present disclosure is administered intravenously in an amount of between about 32.5 mg/m² and about 42.5 mg/m². In some embodiments, the L-asparaginase formulation of the present disclosure is administered intravenously in an amount of between about 21.5 mg/m² and about 38.5 mg/m². In some embodiments, the L-asparaginase formulation of the present disclosure is administered intravenously in an amount of between about 36 mg/m² and about 45 mg/m². In some embodiments, the L-asparaginase formulation of the present disclosure is administered intravenously in an amount of about 37.5 mg/m². In some embodiments, the L-asparaginase formulation of the present disclosure is administered intravenously in an amount of 37.5 mg/m². In some embodiments, the L-asparaginase formulation of the present disclosure is administered intravenously in an amount of 50 mg/m².

In an exemplary embodiment, the L-asparaginase formulation of the present disclosure is administered intravenously in an amount between about 25 mg/m² and about 37.5 mg/m². In an exemplary embodiment, the L-asparaginase formulation of the present disclosure is administered intravenously in an amount between about 25 mg/m² and about 100 mg/m². In an exemplary embodiment, the L-asparaginase formulation of the present disclosure is administered intravenously in an amount between about 25 mg/m² and about 65 mg/m². In an exemplary embodiment, the L-asparaginase formulation of the present disclosure is administered intravenously in an amount between about 25 mg/m² and about 80 mg/m². In an exemplary embodiment, the L-asparaginase formulation of the present disclosure is administered intravenously in an amount between about 30 mg/m² and about 35 mg/m². In an exemplary embodiment, the L-asparaginase formulation of the present disclosure is administered intravenously in an amount between about 30 mg/m² and about 95 mg/m². In an exemplary embodiment, the L-asparaginase formulation of the present disclosure is administered intravenously in an amount between about 35 mg/m² and about 90 mg/m². In an exemplary embodiment, the L-asparaginase formulation of the present disclosure is administered intravenously in an amount between about 40 mg/m² and about 85 mg/m². In an exemplary embodiment, the L-asparaginase formulation of the present disclosure is administered intravenously in an amount between about 45 mg/m² and about 80 mg/m². In an exemplary embodiment, the L-asparaginase formulation of the present disclosure is administered intravenously in an amount between about 50 mg/m² and about 75 mg/m². In an exemplary embodiment, the L-asparaginase formulation of the present disclosure is administered intravenously in an amount between about 55 mg/m² and about 70 mg/m². In an exemplary embodiment, the L-asparaginase formulation of the present disclosure is administered intravenously in an amount between about 60 mg/m² and about 65 mg/m². In an exemplary embodiment, the L-asparaginase formulation of the present disclosure is administered intravenously in an amount between about 30 mg/m² and about 60 mg/m². In an exemplary embodiment, the L-asparaginase formulation of the present disclosure is administered intravenously in an amount between about 35 mg/m² and about 55 mg/m². In an exemplary embodiment, the L-asparaginase formulation of the present disclosure is administered intravenously in an amount between about 40 mg/m² and about 50 mg/m². In an exemplary embodiment, the L-asparaginase formulation of the present disclosure is administered intravenously in an amount between about 42.5 mg/m² and about 57.5 mg/m². In an exemplary embodiment, the L-asparaginase formulation of the present disclosure is administered intravenously in an amount between about 30 mg/m² and about 75 mg/m². In an exemplary embodiment, the L-asparaginase formulation of the present disclosure is administered intravenously in an amount between about 35 mg/m² and about 70 mg/m². In an exemplary embodiment, the L-asparaginase formulation of the present disclosure is administered intravenously in an amount between about 40 mg/m² and about 65 mg/m². In an exemplary embodiment, the L-asparaginase formulation of the present disclosure is administered intravenously in an amount between about 45 mg/m² and about 60 mg/m². In an exemplary embodiment, the L-asparaginase formulation of the present disclosure is administered intravenously in an amount between about 50 mg/m² and about 55 mg/m².

In another embodiment, the method comprises administering a recombinant L-asparaginase of the present disclosure that elicits a lower immunogenic response in a patient compared to a non-recombinant L-asparaginase.

### 2. Dose Frequency

In a specific embodiment, treatment will be administered at a dose ranging from about 1 mg/m² to about 1000 mg/m², typically about 10 mg/m² to about 100 mg/m², at a schedule ranging from about three a week to about once a month, typically once per week or once every other week, as a single agent (e.g., monotherapy) or as part of a combination of chemotherapy drugs, including, but not limited to glucocorticoids, corticosteroids, anticancer compounds or other agents, including, but not limited to methotrexate, dexamethasone, prednisone, prednisolone, vincristine, cyclophosphamide, and anthracycline.

The L-asparaginase formulation of the present disclosure can be administered before, after, or simultaneously with other compounds as part of a multi-agent chemotherapy regimen. In a particular embodiment, the L-asparaginase formulation of the present disclosure comprises a protein recombinantly produced in Pseudomonas fluorescens, and more specifically, the L-asparaginase formulation comprises a sequence according to SEQ ID NO:1. In some embodiments, the L-asparaginase formulation of the present disclosure is administered at a dose that depletes L-asparagine to undetectable levels using methods and apparatus known in the art for a period of about 3 days to about 10 days (e.g., 3, 4, 5, 6, 7, 8, 9, or 10 days) for a single dose.

In some embodiments, the L-asparaginase formulation of the present disclosure is administered three times a week. In some embodiments, the L-asparaginase formulation of the present disclosure is administered every other day over a period of 5 consecutive days followed by a rest period of 2 consecutive days. In some embodiments, the L-asparaginase formulation of the present disclosure is administered on Monday, Wednesday, and Friday of the same week. In some embodiments, the L-asparaginase formulation of the present disclosure is administered three times a week for at least one to three weeks. In some embodiments, the L-asparaginase formulation of the present disclosure is administered every other day over a period of 5 consecutive days followed by a rest period of 2 consecutive days for about one to three weeks. In some embodiments, the L-asparaginase formulation of the present disclosure is administered on Monday, Wednesday, and Friday of the week for about one to three weeks.

In some embodiments, the L-asparaginase formulation of the present disclosure is administered three times a week for about two weeks. In some embodiments, the L-asparaginase formulation of the present disclosure is administered every other day over a period of 5 consecutive days followed by a rest period of 2 consecutive days for about two weeks. In some embodiments, the L-asparaginase formulation of the present disclosure is administered on Monday, Wednesday, and Friday of the same week for about two weeks.

In some embodiments, the L-asparaginase formulation of the present disclosure is administered three times a week for two weeks. In some embodiments, the L-asparaginase formulation of the present disclosure is administered every other day over a period of 5 consecutive days followed by a rest period of 2 consecutive days for two weeks. In some embodiments, the L-asparaginase formulation of the present disclosure is administered on Monday, Wednesday, and Friday of the same week for two weeks.

In some embodiments, the L-asparaginase formulation of the present disclosure is administered three times a week, continuing until the patient no longer has a disease that is treatable by depletion of asparagine. In some embodiments, the L-asparaginase formulation of the present disclosure is administered every other day over a period of 5 consecutive days followed by a rest period of 2 consecutive days, continuing until the patient no longer has a disease that is treatable by depletion of asparagine. In some embodiments, the recombinant L-asparaginase of the present disclosure is administered on Monday, Wednesday, and Friday of the same week, continuing until the patient no longer has a disease that is treatable by depletion of asparagine.

In some embodiments, the L-asparaginase formulation of the present disclosure is administered three times a week, continuing until the patient decides to end or postpone treatment. In some embodiments, the L-asparaginase formulation of the present disclosure is administered every other day over a period of 5 consecutive days followed by a rest period of 2 consecutive days, continuing until the patient decides to end or postpone treatment. In some embodiments, the L-asparaginase formulation of the present disclosure is administered on Monday, Wednesday, and Friday of the same week, continuing until the patient decides to end or postpone treatment.

In some embodiments, the L-asparaginase formulation of the present disclosure is administered about every 48 hours. In some embodiments, the L-asparaginase formulation of the present disclosure is administered every 40 to 58 hours. In some embodiments, the L-asparaginase formulation of the present disclosure is administered about every 42 to 56 hours. In some embodiments, the L-asparaginase formulation of the present disclosure is administered about every 44 to 52 hours. In some embodiments, the L-asparaginase formulation of the present disclosure is administered about every 46 to 50 hours. In some embodiments, the recombinant L-asparaginase of the present disclosure is administered about every 72 hours. In some embodiments, the L-asparaginase formulation of the present disclosure is administered every 64 to 80 hours. In some embodiments, the L-asparaginase formulation of the present disclosure is administered about every 66 to 78 hours. In some embodiments, the L-asparaginase formulation of the present disclosure is administered about every 68 to 76 hours. In some embodiments, the L-asparaginase formulation of the present disclosure is administered about every 70 to 74 hours.

In some embodiments, L-asparaginase formulation of the present disclosure is administered as a second line therapy with patients who are hypersensitive to an E. coli-derived L-asparaginase, and/or may have had a previous hypersensitivity to an Erwinia chrysanthemi-derived L-asparaginase.

In some embodiments, the L-asparaginase formulation is administered to the human subject as a substitute for a dose of a long-acting E. coli-derived asparaginase. In some embodiments, six doses of the L-asparaginase formulation are administered to the human subject as a substitute for one dose of the long-acting E. coli-derived asparaginase. In some embodiments, seven doses of the L-asparaginase formulation are administered to the human subject as a substitute for one dose of the long-acting E. coli-derived asparaginase. In some embodiments, the long-acting E. coli-derived asparaginase is pegaspargase. In a further embodiment, the six separate doses may occur over a period of about two weeks. In another further embodiment, the seven separate doses may occur over a period of about two weeks.

In some embodiments, a dose regimen for the L-asparaginase formulation comprises a cycle, wherein the cycle comprises a first dose, a second dose, and a third dose, wherein the cycle is optionally repeatable, and wherein the first dose, second dose, and third dose are administered about 48-72 hours apart.

In some embodiments, dose amounts may vary within the cycle.

In some embodiments, a dose regimen for the L-asparaginase formulation comprises a cycle, wherein the cycle is optionally repeatable, and wherein the cycle comprises administration of the L-asparaginase formulation every other day over a period of five consecutive days followed by a rest period of two consecutive days, wherein the first dose of the cycle is 25 mg/m², the second dose of the cycle is 25 mg/m² and the third dose of the cycle is 50 mg/m², followed by the rest period of two consecutive days.

In some embodiments, a dose regimen for the L-asparaginase formulation comprises a cycle, wherein the cycle is optionally repeatable, and wherein the cycle comprises administration of the L-asparaginase formulation every other day over a period of five consecutive days followed by a rest period of two consecutive days, wherein the first dose of the cycle is 25 mg/m², the second dose of the cycle is 25 mg/m² and the third dose of the cycle is 37.5 mg/m², followed by the rest period of two consecutive days.

In some embodiments, a dose regimen for the L-asparaginase formulation comprises a cycle, wherein the cycle is optionally repeatable, and wherein the cycle comprises administration of the L-asparaginase formulation every other day over a period of five consecutive days followed by a rest period of two consecutive days, wherein the first dose of the cycle is 37.5 mg/m², the second dose of the cycle is 37.5 mg/m² and the third dose of the cycle is 37.5 mg/m², followed by the rest period of two consecutive days.

In some embodiments, a dose regimen for the L-asparaginase formulation comprises a cycle, wherein the cycle is optionally repeatable, and wherein the cycle comprises administration of the L-asparaginase formulation every other day over a period of five consecutive days followed by a rest period of two consecutive days, wherein the first dose of the cycle is 37.5 mg/m², the second dose of the cycle is 25 mg/m² and the third dose of the cycle is 37.5 in mg/m², followed by the rest period of two consecutive days.

In some embodiments, a dose regimen for the L-asparaginase formulation comprises a cycle, wherein the cycle is optionally repeatable, and wherein the cycle comprises administration of the L-asparaginase formulation every other day over a period of five consecutive days followed by a rest period of two consecutive days, wherein the first dose of the cycle is 37.5 mg/m², the second dose of the cycle is 25 mg/m² and the third dose of the cycle is 25 mg/m², followed by the rest period of two consecutive days. In some embodiments, the first dose of the cycle is administered on a Monday, the second dose of the cycle is given on a Wednesday, and the third dose of the cycle is given on a Friday.

In some embodiments, a dose regimen for the L-asparaginase formulation comprises a cycle, where the cycle is optionally repeatable, and where the cycle comprises administration of the L-asparaginase formulation every other day over a period of five consecutive days followed by a rest period of two consecutive days, where the first dose of the cycle is 25 mg/m², the second dose of the cycle is 25 mg/m² and the third dose of the cycle is 50 mg/m³, followed by the rest period of two consecutive days. In some embodiments, the first dose of the cycle is administered on a Monday, the second dose of the cycle is given on a Wednesday, and the third dose of the cycle is given on a Friday.

In some embodiments, the present disclosure provides a method for depleting asparaginase in a human subject to treat Acute Lymphoblastic Leukemia (ALL) or Lymphoblastic Lymphoma (LBL), the method comprising: on Mondays, Wednesdays, and Fridays, administering intramuscularly about 25 mg/m² of L-asparaginase to the human subject, such that the human subject receives a total of seven doses of L-asparaginase over a two week period.

In some embodiments, the present disclosure provides a method for depleting asparaginase in a human subject to treat Acute Lymphoblastic Leukemia (ALL) or Lymphoblastic Lymphoma (LBL), the method comprising: (a) on Mondays and Wednesdays, administering intramuscularly about 25 mg/m² of L-asparaginase to the human subject, and (b) on Fridays, administering intramuscularly about 50 mg/m² of L-asparaginase to the human subject; such that the human subject receives a total of six doses of L-asparaginase over a two week period.

The dose regimen may encompass any number of cycles for any number of weeks or until any endpoint that is specified herein.

In some embodiments, the L-asparaginase formulation of the present disclosure is administered intravenously in an amount of (i) 25 mg/m² on Monday, (ii) 25 mg/m² on Wednesday, and (iii) 50 mg/m² on Friday.

In some embodiments, the L-asparaginase formulation of the present disclosure is administered intravenously in an amount of 25 mg/m² every 48 hours. In some embodiments, the L-asparaginase formulation of the present disclosure is administered intravenously in an amount of 37.5 mg/m² every 48 hours. In some embodiments, the L-asparaginase formulation of the present disclosure is administered intravenously in an amount of 50 mg/m² every 48 hours.

In some embodiments, the L-asparaginase formulation of the present disclosure is administered intramuscularly in an amount of 25 mg/m² every 48 hours. In some embodiments, the L-asparaginase formulation of the present disclosure is administered intramuscularly in an amount of 37.5 mg/m² every 48 hours. In some embodiments, the L-asparaginase formulation of the present disclosure is administered intramuscularly in an amount of 50 mg/m² every 48 hours.

In some embodiments, in conjunction with embodiments above or below, the L-asparaginase formulation of the present disclosure is administered (i) intravenously in an amount of 25 mg/m² on Monday, (ii) intravenously in an amount of 25 mg/m² on Wednesday, and (iii) intramuscularly in an amount of 50 mg/m² on Friday.

For example, a dosing regimen may comprise administration of six doses (e.g., a first dose, a second dose, a third dose, a fourth dose, a fifth dose, and a sixth dose) of the L-asparaginase formulation over a period of two weeks, wherein during week one, (i) a first dose is administered intravenously or intramuscularly in an amount of 25 mg/m² or 37.5 mg/m² on Monday, (ii) a second dose is administered intravenously or intramuscularly in an amount of 25 mg/m² 37.5 mg/m² on Wednesday, and (iii) a third dose is administered intravenously or intramuscularly in an amount of 25 mg/m² 37.5 mg/m² on Friday; and during week two, (iv) a fourth dose is administered intravenously in an amount of 25 mg/m² on Monday, (v) a fifth dose is administered intravenously in an amount of 25 mg/m² on Wednesday, and (vi) a sixth dose is administered intramuscularly in an amount of 50 mg/m² on Friday.

In some embodiments, a dosing regimen comprises administration of six doses of the L-asparaginase formulation over a period of two weeks, wherein during week one, (i) a first dose is administered intravenously in an amount of 25 mg/m² on Monday, (ii) a second dose is administered intravenously in an amount of 25 mg/m² on Wednesday, and (iii) a third dose is administered intravenously in an amount of 25 mg/m² on Friday; and during week two, (iv) a fourth dose is administered intravenously in an amount of 25 mg/m² on Monday, (v) a fifth dose is administered intravenously in an amount of 25 mg/m² on Wednesday, and (vi) a sixth dose is administered intramuscularly in an amount of 50 mg/m² on Friday.

In some embodiments, a dosing regimen comprises administration of six doses of the L-asparaginase formulation over a period of two weeks, wherein during week one, (i) a first dose is administered intramuscularly in an amount of 25 mg/m² on Monday, (ii) a second dose is administered intramuscularly in an amount of 25 mg/m² on Wednesday, and (iii) a third dose is administered intramuscularly in an amount of 25 mg/m² on Friday; and during week two, (iv) a fourth dose is administered intravenously in an amount of 25 mg/m² on Monday, (v) a fifth dose is administered intravenously in an amount of 25 mg/m² on Wednesday, and (vi) a sixth dose is administered intramuscularly in an amount of 50 mg/m² on Friday.

In some embodiments, a dosing regimen comprises administration of six doses of the L-asparaginase formulation over a period of two weeks, wherein during week one, (i) a first dose is administered intravenously in an amount of 37.5 mg/m² on Monday, (ii) a second dose is administered intravenously in an amount of 37.5 mg/m² on Wednesday, and (iii) a third dose is administered intravenously in an amount of 37.5 mg/m² on Friday; and during week two, (iv) a fourth dose is administered intravenously in an amount of 25 mg/m² on Monday, (v) a fifth dose is administered intravenously in an amount of 25 mg/m² on Wednesday, and (vi) a sixth dose is administered intramuscularly in an amount of 50 mg/m² on Friday.

In some embodiments, a dosing regimen comprises administration of six doses of the L-asparaginase formulation over a period of two weeks, wherein during week one, (i) a first dose is administered intramuscularly in an amount of 37.5 mg/m² on Monday, (ii) a second dose is administered intramuscularly in an amount of 37.5 mg/m² on Wednesday, and (iii) a third dose is administered intramuscularly in an amount of 37.5 mg/m² on Friday; and during week two, (iv) a fourth dose is administered intravenously in an amount of 25 mg/m² on Monday, (v) a fifth dose is administered intravenously in an amount of 25 mg/m² on Wednesday, and (vi) a sixth dose is administered intramuscularly in an amount of 50 mg/m² on Friday.

In some embodiments, a dosing regimen comprise administration of six doses of the L-asparaginase formulation over a period of two weeks, wherein during week one, (i) a first dose is administered intravenously in an amount of 25 mg/m² on Monday, (ii) a second dose is administered intravenously in an amount of 25 mg/m² on Wednesday, and (iii) a third dose is administered intravenously in an amount of 50 mg/m² on Friday; and during week two, (iv) a fourth dose is administered intravenously in an amount of 25 mg/m² on Monday, (v) a fifth dose is administered intravenously in an amount of 25 mg/m² on Wednesday, and (vi) a sixth dose is administered intramuscularly in an amount of 50 mg/m² on Friday.

### Kits

In one aspect, provided herein is a kit, comprising: (i) any one of the formulations described herein; and (ii) instructions for treating a disease, condition, or disorder that is treatable by asparagine depletion in a subject in need thereof.

In some embodiments, the kit comprises a formulation, comprising: (i) an L-asparaginase, wherein the L-asparaginase comprises four monomer units, wherein each monomer unit has an amino acid sequence that is at least about 70% identical to SEQ ID NO: 1, and (ii) one or more stabilizers, or one or more buffers, or any combination thereof. In some embodiments, the L-asparaginase formulation comprises less than about 0.6% low-molecular-weight (LMW) species after storage at 40° ° C. for two months. In some embodiments, the L-asparaginase formulation comprises less than 2% high-molecular-weight (HMW) species after storage at 40° C. for two months. In some embodiments, the L-asparaginase formulation comprises less than about 0.6% low-molecular-weight (LMW) species and less than 2% high-molecular-weight (HMW) species after storage at 40° C. for two months. In some embodiments, the L-asparaginase formulation comprises less than about 0.6% low-molecular-weight (LMW) species after storage at 37° ° C. for one week.

In some embodiments, the kit comprises a formulation, comprising (i) an L-asparaginase, wherein the L-asparaginase comprises four monomer units, wherein each monomer unit has an amino acid sequence that is at least about 95% identical to SEQ ID NO: 1; (ii) one or more disaccharides, wherein the one or more disaccharides comprise trehalose, sucrose, or any combination thereof; and (iii) one or more buffers, wherein the one or more buffers are substantially free of amino acid, wherein the formulation comprises less than about 5% low-molecular-weight (LMW) species after storage at 37° ° C. for one week.

In some embodiments, the kit comprises a formulation, comprising (i) an L-asparaginase, wherein the L-asparaginase comprises four monomer units, wherein each monomer unit has an amino acid sequence that is at least about 70% identical to SEQ ID NO: 1, and wherein the L-asparaginase is present at a concentration of around 20 mg/mL; (ii) trehalose, wherein the trehalose is present at a concentration of about 170 mM; (iii) sodium phosphate, wherein the sodium phosphate is present at a concentration of about 20 mM; (iv) sodium chloride, wherein the sodium chloride is present at a concentration of about 50 mM; and (v) polysorbate 80, wherein the polysorbate 80 is present at a concentration of about 0.02% (w/v), wherein the formulation comprises less than about 0.6% low-molecular-weight (LMW) species after storage at 37° ° C. for one week. In some embodiments, the formulation has a pH of between about 4.0 and about 8.5. In some embodiments, the formulation has a pH of about 7.0.

In some embodiments, the kit comprises a formulation, comprising (i) an L-asparaginase, wherein the L-asparaginase comprises four monomer units, wherein each monomer unit has an amino acid sequence that is at least about 95% identical to SEQ ID NO: 1, and wherein the L-asparaginase is present at a concentration of around 20 mg/ml; (ii) trehalose, wherein the trehalose is present at a concentration of about 170 mM; (iii) sodium phosphate, wherein the sodium phosphate is present at a concentration of about 20 mM; (iv) sodium chloride, wherein the sodium chloride is present at a concentration of about 50 mM; and (v) polysorbate 80, wherein the polysorbate 80 is present at a concentration of about 0.02% (w/v), wherein the formulation comprises less than about 5% low-molecular-weight (LMW) species after storage at 37° C. for one week. In some embodiments, the formulation has a pH of between about 4.0 and about 8.5. In some embodiments, the formulation has a pH of about 7.0.

In some embodiments of the foregoing, the kits comprise instructions for treating cancer. In some embodiments, the kits comprise instructions for treating acute lymphoblastic leukemia (ALL). In some embodiments, the ALL is relapsed. In some embodiments, the kits comprise instructions for treating lymphoblastic lymphoma (LBL). In some embodiments, the LBL is relapsed.

### Examples

### Nessler Activity

Asparaginase activity was measured by Nessler assay, in which the measurement of asparaginase activity is based on an endpoint limit assay in which the enzyme is incubated at +37° ° C. under saturating L-asparagine concentration for 15 minutes. The reaction is stopped by addition of Nessler's reagent and the amount of ammonia produced during the reaction is assessed colorimetrically (at 450 nm) using a calibration curve constructed from known quantities of ammonium sulfate.

### Size Exclusion Chromatography (SEC)

The column was conditioned with mobile phase (50 mM phosphate, 200 mM NaCl, pH 7.0) at 1 mL/min for 1 hour before injections. BioRad Gel Filtration Standards were diluted 10-fold in blue dextran for a total volume of 1 mL. Samples and reference standards were diluted to 1 mg/mL in mobile phase and injected after multiple injections of mobile phase blanks to clear the column.

Note that, in the various Tables in the Examples below, the entry “ND” indicates that a particular data point was not determined.

### Example 1: Baseline Buffer/Excipient Screen

The effects of pH and buffer type from six buffer/pH combinations in the range of 4.5-8.0 were assessed on stability of L-asparaginase. An additional set of formulations with 6 different excipients in either 20 mM histidine buffer at pH 7.0 or 20 mM sodium phosphate at pH 8.0 were prepared as an initial excipient screen (Table 1). L-asparaginase (1.0 mL of Lot RM-LAP-P03/P05/P06 Pool of Bulks at 20.9 mg/mL) was buffer exchanged using centrifugal ultrafiltration devices (Amicon Ultra-4 10k MWCO) with 100 mL of the corresponding formulation buffer. Following the final buffer-exchange, the protein concentration was measured, and the samples were adjusted to approximately 20 mg/mL. The formulated samples were split into two aliquots, and a one week stability study was performed with the formulated RC at 37° C. and 5° C. The formulations were analyzed in parallel at the end of the one week storage.

TABLE 1

| | | | Mea- | Measured |
|---|---|---|---|---|
| | | | sured | Concentration |
| Buffer | pH | Excipient | pH | (mg/mL) |
| Acetate (20 mM) | 4.5 | N/A | 4.6 | 22 |
| | 5.5 | | 5.6 | 22 |
| Succinate (20 mM) | 4.5 | | 4.6 | 18 |
| | 5.5 | | 5.6 | 19 |
| | 6.5 | | 6.5 | 20 |
| Sodium Citrate | 5.0 | | 5.1 | 22 |
| (20 mM) | 6.0 | | 6.0 | 22 |
| | 7.0 | | 6.9 | 20 |
| Histidine (20 mM) | 6.0 | | 6.0 | 21 |
| | 7.0 | | 7.1 | 20 |
| Phosphate (20 mM) | 6.5 | | 6.6 | 21 |
| | 7.5 | | 7.4 | 19 |
| | 8.0 | | 7.9 | 18 |
| Tris (20 mM) | 8.5 | | 8.5 | 19 |
| 20 mM Histidine | 7.0 | 3% (w/v) Proline | 7.1 | 19 |
| | | 150 mM NaCl | 6.9 | 20 |
| | | 150 mM Arginine | 6.9 | 18 |
| | | 250 mM Trehalose | 7.1 | 19 |
| | | 250 mM Sorbitol | 7.1 | 22 |
| | | 250 mM Sucrose | 6.96 | 21 |
| 20 mM Phosphate | 8.0 | 150 mM Lysine | 7.97 | 22 |
| | | 150 mM NaCl | 7.89 | 22 |
| | | 150 mM Arginine | 7.88 | 18 |
| | | 250 mM Trehalose | 7.90 | 18 |
| | | 250 mM Sorbitol | 7.87 | 22 |
| | | 250 mM Sucrose | 7.85 | 19 |

The purpose of the excipient screen was to evaluate acetate, succinate, citrate, histidine, phosphate, and tris buffers from pH 4.5 to 8.5. Additionally, the excipients proline, lysine, sodium chloride, arginine, trehalose, sorbitol, and sucrose in histidine buffer at pH 7.0 or phosphate buffer at pH 8.0 were evaluated for the influence of excipients. SEC and the Nessler activity assay were used to assess the stability of RC in the presence of these excipients in the selected buffers.

The purity of 26 formulations was assessed by SEC-HPLC. Results are summarized in Table 2 and Table 3. Little difference was observed in the SEC profiles between the 5° C. and 37° C. conditions after 1 week storage for all formulations. However, differences were observed between the different formulations. The main peak purities had a range of 66.4-98.3%. The histidine buffer formulation all had purity levels between 66.4% and 74.0%, while the remaining formulations had purity levels between 90.6% and 98.3%. In the histidine, succinate, citrate, phosphate with lysine, and phosphate with arginine buffers, a LMW species was observed at ˜11.6 minutes ranging from 1.3-32.4%. The formulations in acetate, phosphate, and tris buffers showed between 0.2-0.6% LMW species. Formulations in phosphate buffer with sodium chloride, trehalose, sorbitol, and sucrose showed similar levels of LMW species to the formulations without excipients.

TABLE 2

| | | | Average | Average | Average |
|---|---|---|---|---|---|
| | | | HMW | Main Peak | LMW |
| | | Condition | Percentage | Percentage | Percentage |
| Buffer | pH | (° C.) | (%) | (%) | (%) |
| 20 mM Acetate | 4.5 | 5 | 1.8 | 97.7 | 0.5 |
| | | 37 | 1.5 | 97.9 | 0.6 |
| | 5.5 | 5 | 1.7 | 97.7 | 0.6 |
| | | 37 | 1.7 | 97.8 | 0.5 |
| 20 mM | 4.5 | 5 | 1.7 | 96.8 | 1.5 |
| Succinate | | 37 | 1.5 | 96.9 | 1.6 |
| | 5.5 | 5 | 1.8 | 96.8 | 1.3 |
| | | 37 | 1.6 | 96.0 | 2.4 |
| | 6.5 | 5 | 1.8 | 96.8 | 1.4 |
| | | 37 | 1.7 | 96.3 | 2.0 |
| 20 mM Sodium | 5.0 | 5 | 1.8 | 95.3 | 2.8 |
| Citrate | | 37 | 1.6 | 95.5 | 2.9 |
| | 6.0 | 5 | 1.8 | 95.2 | 3.1 |
| | | 37 | 1.6 | 95.2 | 3.2 |
| | 7.0 | 5 | 1.8 | 95.7 | 2.5 |
| | | 37 | 1.7 | 95.7 | 2.6 |
| 20 mM | 6.0 | 5 | 1.3 | 71.7 | 27.0 |
| Histidine | | 37 | 1.1 | 72.7 | 26.1 |
| | 7.0 | 5 | 1.3 | 70.7 | 28.0 |
| | | 37 | 1.0 | 72.2 | 26.8 |
| 20 mM | 6.5 | 5 | 1.8 | 97.9 | 0.3 |
| Phosphate | | 37 | 1.6 | 98.1 | 0.2 |
| | 7.5 | 5 | 1.8 | 97.8 | 0.4 |
| | | 37 | 1.6 | 98.2 | 0.2 |
| | 8.0 | 5 | 1.8 | 97.8 | 0.3 |
| | | 37 | 1.5 | 98.2 | 0.3 |
| 20 mM Tris | 8.5 | 5 | 1.8 | 97.9 | 0.3 |
| | | 37 | 1.5 | 98.3 | 0.2 |

TABLE 3

| | | | | Average | |
|---|---|---|---|---|---|
| | | | Average | Main | Average |
| | | | HMW | Peak | LMW |
| | | Condition | Percentage | Percentage | Percentage |
| Buffer | Excipient | (° C.) | (%) | (%) | (%) |
| 20 mM | 3% (w/v) | 5 | 1.2 | 67.9 | 30.9 |
| Histidine, | Proline | 37 | 1.0 | 68.4 | 30.5 |
| pH 7.0 | 150 mM | 5 | 1.4 | 69.0 | 29.6 |
| | NaCl | 37 | 1.1 | 69.7 | 29.2 |
| | 150 mM | 5 | 1.3 | 66.4 | 32.4 |
| | Arginine | 37 | 1.1 | 66.9 | 32.0 |
| | 250 mM | 5 | 1.2 | 68.2 | 30.7 |
| | Trehalose | 37 | 1.0 | 68.7 | 30.3 |
| | 250 mM | 5 | 1.3 | 71.1 | 27.6 |
| | Sorbitol | 37 | 1.0 | 74.0 | 25.0 |
| | 250 mM | 5 | 1.2 | 71.4 | 27.4 |
| | Sucrose | 37 | 1.1 | 71.8 | 27.2 |
| 20 mM | 150 mM | 5 | 1.7 | 95.9 | 2.4 |
| Phosphate, | Lysine | 37 | 1.4 | 96.4 | 2.2 |
| pH 8.0 | 150 mM | 5 | 1.9 | 97.8 | 0.3 |
| | NaCl | 37 | 1.5 | 98.3 | 0.2 |
| | 150 mM | 5 | 1.7 | 90.6 | 7.7 |
| | Arginine | 37 | 1.3 | 91.2 | 7.5 |
| | 250 mM | 5 | 1.8 | 97.9 | 0.3 |
| | Trehalose | 37 | 1.8 | 98.0 | 0.2 |
| | 250 mM | 5 | 1.9 | 97.8 | 0.3 |
| | Sorbitol | 37 | 1.7 | 98.1 | 0.2 |
| | 250 mM | 5 | 1.8 | 97.9 | 0.3 |
| | Sucrose | 37 | 1.6 | 98.1 | 0.3 |

Based on the highest purity and lowest LMW species percentage, acetate and phosphate buffer were selected for further evaluation. Additionally, trehalose, sorbitol, and sodium chloride were chosen as the excipients to be evaluated in the DOE to assess their impact on formulation stability under more stringent stress conditions.

### Example 2: DOE

A statistical design of experiment (DOE) approach considering one numeric factor (pH) and one categorical factor (excipient) was used to evaluate the effects of pH and excipient for acetate and phosphate buffer in the range of 4.5-5.5 and 7.0-8.0, respectively, on L-asparaginase. In order to accommodate the non-overlapping ranges of the two buffer systems, acetate and phosphate, two independent DOEs using a linear one-factor design were performed for each buffer system in parallel. The experimental design was generated using Stat Ease, Inc. Design-Expert® Version 9.0.6.2. Significance of the responses were evaluated at 95% confidence level.

An additional set of off-design formulations evaluated the effect of the three selected excipients using Tris buffer at pH 8.5 and the effect of addition of PS-80 to acetate pH 5.3 and phosphate pH 7.5 DOEs with sorbitol and trehalose. The formulations were exposed to a thermal stress, 40° C. for 2 weeks, and a single Freeze/Thaw cycle.

The Nessler activity assay for formulations exposed to a single freeze/thaw cycle or stored for 2 weeks at 40° ° C. had activities that ranged from 600 to 1270 μmol/min·mgE (Table 4 and Table 5). No significant loss of activity of the formulations was observed the thermal or freeze/thaw stress, and it is undetermined if the formulations with increased activity is a result of the formulation buffer or the variability of the Nessler Activity assay. The formulations were analyzed by RP-HPLC. By RP-HPLC, all formulations in both acetate and phosphate buffer were determined to be 100% main peak after a single freeze/thaw stress, and 98% main peak and 2% minor peak after 2 weeks at 40° C.

TABLE 4

| | | | Enzyme Activity | Enzyme Activity |
|---|---|---|---|---|
| | | | (μmol/min · mgE) | (μmol/min · mgE) |
| Buffer | pH | Excipient | Freeze/Thaw | 40° C. 75% RH |
| 20 mM | 7.00 | 250 mM | 726 | 831 |
| Phosphate | | Sorbitol | 639 | 600 |
| | 7.50 | | 773 | 1270 |
| | 8.00 | | 718 | 748 |
| | | | 784 | 883 |
| | 7.00 | 250 mM | 675 | 862 |
| | | Trehalose | 759 | 1104 |
| | 7.50 | | 744 | 723 |
| | 8.00 | | 800 | 800 |
| | | | 747 | 1149 |
| | 7.00 | 150 mM | 788 | 990 |
| | | NaCl | 853 | 880 |
| | 7.50 | | 873 | 985 |
| | 8.00 | | 740 | 878 |
| | | | 771 | 758 |
| 20 mM | 4.50 | 250 mM | 808 | 948 |
| Acetate | | Sorbitol | 663 | 932 |
| | 5.00 | | 809 | 796 |
| | 5.50 | | 719 | 732 |
| | | | 812 | 887 |
| | 4.50 | 250 mM | 867 | 789 |
| | | Trehalose | 718 | 800 |
| | 5.00 | | 722 | 676 |
| | 5.50 | | 691 | 879 |
| | | | 753 | 851 |
| | 4.50 | 150 mM | 837 | 826 |
| | | NaCl | 903 | 821 |
| | 5.00 | | 1001 | 763 |
| | 5.50 | | 806 | 826 |
| | | | 822 | 841 |

TABLE 5

| | | | | Enzyme Activity | Enzyme Activity |
|---|---|---|---|---|---|
| | | | | (μmol/min · mgE) | (μmol/min · mgE) |
| Buffer | pH | Excipient | Surfactant | Freeze/Thaw | 40° C. 75% RH |
| 20 mM Tris | 8.50 | 250 mM Trehalose | — | 702 | 835 |
| | | 250 mM Sorbitol | | 824 | 771 |
| | | 150 mM NaCl | | 830 | 709 |
| 20 mM Phosphate | 7.50 | 250 mM Sorbitol | 0.02% PS- | 744 | |
| | | 250 mM Trehalose | 80 | 673 | |
| 20 mM Acetate | 5.25 | 250 mM Sorbitol | | 618 | |
| | | 250 mM Trehalose | | 921 | |

All formulations and stress conditions were analyzed by SEC-HPLC. Results are shown in Table 6. After thermal stress, the purity in phosphate buffered formulations ranged from 88.70% to 99.31%, the HMW peak ranged from 0.66% to 1.00%, the LMW shoulder ranged from none detected to 10.98%, and the LMW peak was none detected. At pH 7.0, all three excipients show similar SEC profiles after thermal stress, and the main peak percentage was 99% for each excipient.

TABLE 6

| | | | HMW | Main | LMW | LMW |
|---|---|---|---|---|---|---|
| | | | Peak | Peak | Shoulder | Peak |
| PH | Excipient | Condition | (%) | (%) | (%) | (%) |
| 7.0 | 250 mM | Freeze/Thaw | 0.68 | 99.32 | ND | ND |
| | Sorbitol | 40° C., 2 W | 0.99 | 99.01 | ND | ND |
| | | Freeze/Thaw | 0.71 | 99.29 | ND | ND |
| | | 40° C., 2 W | 1.00 | 99.00 | ND | ND |
| 7.5 | | Freeze/Thaw | 0.66 | 99.34 | ND | ND |
| | | 40° C., 2 W | 0.89 | 99.11 | ND | ND |
| 8.0 | | Freeze/Thaw | 0.71 | 99.29 | ND | ND |
| | | 40°C., 2 W | 0.98 | 90.33 | 8.68 | ND |
| | | Freeze/Thaw | 0.66 | 99.34 | ND | ND |
| | | 40° C., 2 W | 0.94 | 90.12 | 8.94 | ND |
| 7.0 | 250 mM | Freeze/Thaw | 0.66 | 99.34 | ND | ND |
| | Trehalose | 40° C., 2 W | 0.76 | 99.24 | ND | ND |
| | | Freeze/Thaw | 0.66 | 98.05 | ND | 1.28 |
| | | 40° C., 2 W | 0.75 | 99.25 | ND | ND |
| 7.5 | | Freeze/Thaw | 0.68 | 98.47 | ND | 0.85 |
| | | 40° C., 2 W | 0.80 | 92.44 | 6.76 | ND |
| 8.0 | | Freeze/Thaw | 0.68 | 99.32 | ND | ND |
| | | 40° C., 2 W | 0.82 | 91.09 | 8.09 | ND |
| | | Freeze/Thaw | 0.68 | 98.44 | ND | 0.88 |
| | | 40° C., 2 W | 0.81 | 91.36 | 7.83 | ND |
| 7.0 | 150 mM | Freeze/Thaw | 0.68 | 99.32 | ND | ND |
| | NaCl | 40° C., 2 W | 0.69 | 99.31 | ND | ND |
| | | Freeze/Thaw | 0.69 | 99.31 | ND | ND |
| | | 40° C., 2 W | 0.73 | 99.27 | ND | ND |
| 7.5 | | Freeze/Thaw | 0.68 | 99.32 | ND | ND |
| | | 40° C., 2 W | 0.73 | 92.00 | 7.27 | ND |
| 8.0 | | Freeze/Thaw | 0.68 | 99.32 | ND | ND |
| | | 40° C., 2 W | 0.72 | 88.29 | 10.98 | ND |
| | | Freeze/Thaw | 0.66 | 99.34 | ND | ND |
| | | 40° C., 2 W | 0.66 | 88.70 | 10.64 | ND |

The decrease in main peak purity and increase in LMW shoulder percentage were fit to a linear model dependent on pH only with the maximum main peak purity observed at pH 7.0 (Table 7). The off-design formulations at pH 8.5 also show formation of significant quantities of LMW shoulder after thermal stress (Table 8). The formation of HIMW species was found to be dependent on excipient, but not pH, with sorbitol having the overall largest percentage of HMW species.

TABLE 7

| | Sum of | | Mean | F | p-value |
|---|---|---|---|---|---|
| Source | Squares | df | Square | Value | Prob > F |
| Model | 253.83 | 1 | 253.83 | 83.28 | <0.0001 |
| A-pH | 253.83 | 1 | 253.83 | 83.28 | <0.0001 |
| Residual | 39.62 | 13 | 3.05 | | |

TABLE 8

| | | | | | HMW | Main | LMW | LMW |
|---|---|---|---|---|---|---|---|---|
| | | | | | Peak | Peak | Shoulder | Peak |
| Buffer | PH | Excipient | Surfactant | Condition | (%) | (%) | (%) | (%) |
| 20 mM | 8.5 | 250 mM | — | Freeze/Thaw | 0.66 | 99.34 | ND | ND |
| Tris | | Trehalose | | 40° C., 2 W | 0.71 | 90.41 | 8.88 | ND |
| | | 250 mM | | Freeze/Thaw | 0.67 | 99.33 | ND | ND |
| | | Sorbitol | | 40° C., 2 W | 0.79 | 90.18 | 9.04 | ND |
| | | 150 mM | | Freeze/Thaw | 0.68 | 99.32 | ND | ND |
| | | NaCl | | 40°C., 2 W | 0.64 | 86.75 | 12.61 | ND |
| 20 mM | 7.5 | 250 mM | 0.02% | Freeze/Thaw | 0.91 | 99.09 | ND | ND |
| Phosphate | | Sorbitol | PS-80 | | | | | |
| | | 250 mM | | Freeze/Thaw | 0.89 | 99.11 | ND | ND |
| | | Trehalose | | | | | | |
| 20 mM | 5.3 | 250 mM | | Freeze/Thaw | 0.69 | 97.62 | ND | 1.69 |
| Acetate | | Sorbitol | | | | | | |
| | | 250 mM | | Freeze/Thaw | 0.6 | 97.77 | ND | 1.63 |
| | | Trehalose | | | | | | |

Under freeze/thaw stress for the sodium phosphate formulations, the SEC-HPLC main peak purity range from 98.05% to 99.34%, the HMW peak ranged from 0.66% to 0.71%, and the LMW peak ranged from none detected to 1.28%. No trends were found for SEC under freeze/thaw stress, but the LMW peak was only observed in trehalose formulations.

Under freeze/thaw stress for the sodium acetate formulations, the SEC-HPLC main peak purity range from 97.69% to 98.07%, the HMW peak ranged from 0.54% to 0.69%, and the LMW peak ranged from 1.34% to 1.62%. Results are show in Table 9. No trends were found for SEC under freeze/thaw stress. Under thermal stress, the acetate formulations purity ranged from 97.65% to 98.02%, the HMW peak ranged from 0.46% to 0.68%, and the LMW peak ranged from 1.52% to 1.82%. The small differences in HMW percentage were found to be dependent on pH and excipient with higher HMW species at pH 5.5 and more for sodium chloride over trehalose or sorbitol.

TABLE 9

| | | | HMW | Main | LMW | LMW |
|---|---|---|---|---|---|---|
| | | | Peak | Peak | Shoulder | Peak |
| pH | Excipient | Condition | (%) | (%) | (%) | (%) |
| 4.5 | 250 mM | Freeze/Thaw | 0.61 | 98.05 | ND | 1.34 |
| | Sorbitol | 40° C., 2 W | 0.49 | 97.84 | ND | 1.67 |
| | | Freeze/Thaw | 0.58 | 98.07 | ND | 1.35 |
| | | 40° C., 2 W | 0.46 | 97.94 | ND | 1.60 |
| 5.0 | | Freeze/Thaw | 0.54 | 98.06 | ND | 1.41 |
| | | 40° C., 2 W | 0.49 | 97.85 | ND | 1.66 |
| 5.5 | | Freeze/Thaw | 0.56 | 98.00 | ND | 1.44 |
| | | 40° C.. 2 W | 0.57 | 97.83 | ND | 1.59 |
| | | Freeze/Thaw | 0.55 | 98.01 | ND | 1.44 |
| | | 40º C., 2 W | 0.56 | 97.91 | ND | 1.53 |
| 4.5 | 250 mM | Freeze/Thaw | 0.59 | 98.06 | ND | 1.35 |
| | Trehalose | 40° C., 2 W | 0.47 | 97.85 | ND | 1.68 |
| | | Freeze/Thaw | 0.58 | 98.05 | ND | 1.37 |
| | | 40° C., 2 W | 0.46 | 98.02 | ND | 1.52 |
| 5.0 | | Freeze/Thaw | 0.55 | 98.02 | ND | 1.43 |
| | | 40° C., 2 W | 0.49 | 97.79 | ND | 1.71 |
| 5.5 | | Freeze/Thaw | 0.56 | 98.00 | ND | 1.44 |
| | | 40° C., 2 W | 0.54 | 97.82 | ND | 1.64 |
| | | Freeze/Thaw | 0.55 | 98.03 | ND | 1.42 |
| | | 40° C., 2 W | 0.51 | 97.86 | ND | 1.63 |
| 4.5 | 150 mM | Freeze/Thaw | 0.63 | 97.94 | ND | 1.42 |
| | NaCl | 40° C., 2 W | 0.54 | 97.88 | ND | 1.58 |
| | | Freeze/Thaw | 0.63 | 97.90 | ND | 1.46 |
| | | 40° C., 2 W | 0.53 | 97.65 | ND | 1.82 |
| 5.0 | | Freeze/Thaw | 0.67 | 97.90 | ND | 1.43 |
| | | 40° C., 2 W | 0.61 | 97.76 | ND | 1.63 |
| 5.5 | | Freeze/Thaw | 0.68 | 97.83 | ND | 1.49 |
| | | 40° C., 2 W | 0.68 | 97.74 | ND | 1.58 |
| | | Freeze/Thaw | 0.69 | 97.69 | ND | 1.62 |
| | | 40° C., 2 W | 0.67 | 97.72 | ND | 1.60 |

All formulations and stress conditions were analyzed by IEX-HPLC. After thermal stress, the phosphate buffer formulations showed a main peak purity range from 52.9% to 63.1%, the acidic species from 9.3% to 18.8%, and basic species from 25.2% to 30.1%. The amount of acidic species increased, while the main peak decreased with increasing pH. The main peak percentage was also dependent on the excipient type. Results are shown in Table 10 and Table 11.

TABLE 10

| | | | HMW | Main | LMW | LMW |
|---|---|---|---|---|---|---|
| | | | Peak | Peak | Shoulder | Peak |
| pH | Excipient | Condition | (%) | (%) | (%) | (%) |
| 4.5 | 250 mM | Freeze/Thaw | 0.61 | 98.05 | ND | 1.34 |
| | Sorbitol | 40° C., 2 W | 0.49 | 97.84 | ND | 1.67 |
| | | Freeze/Thaw | 0.58 | 98.07 | ND | 1.35 |
| | | 40° C., 2 W | 0.46 | 97.94 | ND | 1.60 |
| 5.0 | | Freeze/Thaw | 0.54 | 98.06 | ND | 1.41 |
| | | 40° C., 2 W | 0.49 | 97.85 | ND | 1.66 |
| 5.5 | | Freeze/Thaw | 0.56 | 98.00 | ND | 1.44 |
| | | 40° C., 2 W | 0.57 | 97.83 | ND | 1.59 |
| | | Freeze/Thaw | 0.55 | 98.01 | ND | 1.44 |
| | | 40° C., 2 W | 0.56 | 97.91 | ND | 1.53 |
| 4.5 | 250 mM | Freeze/Thaw | 0.59 | 98.06 | ND | 1.35 |
| | Trehalose | 40° C., 2 W | 0.47 | 97.85 | ND | 1.68 |
| | | Freeze/Thaw | 0.58 | 98.05 | ND | 1.37 |
| | | 40° C., 2 W | 0.46 | 98.02 | ND | 1.52 |
| 5.0 | | Freeze/Thaw | 0.55 | 98.02 | ND | 1.43 |
| | | 40º C., 2 W | 0.49 | 97.79 | ND | 1.71 |
| 5.5 | | Freeze/Thaw | 0.56 | 98.00 | ND | 1.44 |
| | | 40° C., 2 W | 0.54 | 97.82 | ND | 1.64 |
| | | Freeze/Thaw | 0.55 | 98.03 | ND | 1.42 |
| | | 40° C., 2 W | 0.51 | 97.86 | ND | 1.63 |
| 4.5 | 150 mM | Freeze/Thaw | 0.63 | 97.94 | ND | 1.42 |
| | NaCl | 40° C., 2 W | 0.54 | 97.88 | ND | 1.58 |
| | | Freeze/Thaw | 0.63 | 97.90 | ND | 1.46 |
| | | 40° C., 2 W | 0.53 | 97.65 | ND | 1.82 |
| 5.0 | | Freeze/Thaw | 0.67 | 97.90 | ND | 1.43 |
| | | 40° C., 2 W | 0.61 | 97.76 | ND | 1.63 |
| 5.5 | | Freeze/Thaw | 0.68 | 97.83 | ND | 1.49 |
| | | 40° C., 2 W | 0.68 | 97.74 | ND | 1.58 |
| | | Freeze/Thaw | 0.69 | 97.69 | ND | 1.62 |
| | | 40° C., 2 W | 0.67 | 97.72 | ND | 1.60 |

TABLE 11

| | | | Acidic | Main | Basic | Main |
|---|---|---|---|---|---|---|
| | | | Species | Peak | Species | Peak RT |
| Buffer | pH | Excipient | (%) | (%) | (%) | (min) |
| 20 mM | 7.0 | 250 mM | 13.5 | 57.5 | 29.0 | 18.6 |
| Phosphate | | Sorbitol | 13.3 | 57.2 | 29.5 | 18.6 |
| | 7.5 | | 15.3 | 55.7 | 29.0 | 18.6 |
| | 8.0 | | 18.6 | 53.2 | 28.2 | 18.6 |
| | | | 18.8 | 52.9 | 28.3 | 18.6 |
| | 7.0 | 250 mM | 10.6 | 59.3 | 30.1 | 18.7 |
| | | Trehalose | 10.3 | 59.9 | 29.9 | 18.7 |
| | 7.5 | | 12.8 | 57.8 | 29.4 | 18.6 |
| | 8.0 | | 15.0 | 55.7 | 29.3 | 18.6 |
| | | | 17.7 | 57.0 | 25.2 | 18.6 |
| | 7.0 | 150 mM | 9.5 | 63.1 | 27.3 | 18.7 |
| | | NaCl | 9.3 | 62.1 | 28.6 | 18.6 |
| | 7.5 | | 11.9 | 59.2 | 28.9 | 18.7 |
| | 8.0 | | 13.8 | 56.6 | 29.6 | 18.7 |
| | | | 15.5 | 56.1 | 28.4 | 18.7 |
| 20 mM | 4.5 | 250 mM | 6.5 | 54.6 | 38.8 | 18.7 |
| Acetate | | Sorbitol | 6.2 | 54.1 | 39.7 | 18.7 |
| | 5.0 | | 6.1 | 54.0 | 39.9 | 18.7 |
| | 5.5 | | 6.6 | 55.2 | 38.2 | 18.7 |
| | | | 6.5 | 55.3 | 38.2 | 18.7 |
| | 4.5 | 250 mM | 6.4 | 53.6 | 40.0 | 18.7 |
| | | Trehalose | 6.3 | 54.5 | 39.2 | 19.3 |
| | 5.0 | | 6.2 | 55.2 | 38.6 | 19.3 |
| | 5.5 | | 6.2 | 56.3 | 37.5 | 19.3 |
| | | | 5.9 | 53.3 | 40.8 | 19.2 |
| | 4.5 | 150 mM | 7.3 | 51.4 | 41.3 | 19.2 |
| | | NaCl | 7.1 | 51.3 | 41.6 | 19.2 |
| | 5.0 | | 6.2 | 52.1 | 41.8 | 19.2 |
| | 5.5 | | 6.3 | 56.4 | 37.2 | 19.2 |
| | | | 6.2 | 54.1 | 39.7 | 19.2 |

At pH 7.0, the main peak percentage was 62.1-63.1%, 59.3-59.9%, and 57.2-57.5% for sodium chloride, trehalose, and sorbitol, respectively. No trend was observed for the amount of basic species under thermal stress. A similar trend for pH was observed for the main peak and acidic species percentages for these formulations under freeze/thaw stress (Table 12), but little difference was observed between the excipients.

TABLE 12

| | | | Acid | Mean | Basic | Main |
|---|---|---|---|---|---|---|
| | | | Species | Peak | Species | Peak |
| Buffer | pH | Excipient | (%) | (%) | (%) | (RT) |
| 20 mM | 7.0 | 250 mM | 7.8 | 69.5 | 22.7 | 19.2 |
| Phosphate | | Sorbitol | 7.5 | 69.4 | 23.1 | 19.2 |
| | 7.5 | | 8.6 | 68.5 | 22.9 | 19.2 |
| | 8.0 | | 10.4 | 67.4 | 22.2 | 19.2 |
| | | | 10.0 | 67.2 | 22.8 | 19.2 |
| | 7.0 | 250 mM | 7.5 | 69.1 | 23.4 | 19.2 |
| | | Trehalose | 7.3 | 69.5 | 23.2 | 19.2 |
| | 7.5 | | 9.2 | 64.1 | 26.7 | 19.3 |
| | 8.0 | | 9.6 | 65.3 | 25.1 | 19.3 |
| | | | 10.9 | 67.4 | 21.7 | 19.3 |
| | 7.0 | 150 mM | 6.9 | 68.6 | 24.5 | 19.3 |
| | | NaCl | 7.1 | 68.6 | 24.3 | 19.3 |
| | 7.5 | | 8.6 | 66.9 | 24.5 | 19.2 |
| | 8.0 | | 8.7 | 66.8 | 24.5 | 19.2 |
| | | | 10.1 | 66.4 | 23.5 | 19.3 |
| 20 mM | 4.5 | 250 mM | 5.5 | 67.7 | 26.8 | 19.2 |
| Acetate | | Sorbitol | 5.5 | 67.7 | 26.7 | 19.2 |
| | 5.0 | | 5.7 | 68.6 | 25.7 | 19.2 |
| | 5.5 | | 5.8 | 69.0 | 25.3 | 19.2 |
| | | | 5.8 | 69.4 | 24.8 | 19.2 |
| | 4.5 | 250 mM | 5.6 | 69.7 | 24.7 | 19.2 |
| | | Trehalose | 5.6 | 69.7 | 24.7 | 19.2 |
| | 5.0 | | 5.5 | 70.2 | 24.3 | 19.2 |
| | 5.5 | | 5.5 | 70.4 | 24.1 | 19.2 |
| | | | 5.7 | 69.8 | 24.6 | 19.2 |
| | 4.5 | 150 mM | 5.4 | 70.4 | 24.1 | 19.2 |
| | | NaCl | 5.5 | 70.4 | 24.1 | 19.2 |
| | 5.0 | | 5.5 | 70.5 | 24.0 | 19.2 |
| | 5.5 | | 5.6 | 70.6 | 23.9 | 19.2 |
| | | | 5.6 | 70.4 | 24.0 | 19.2 |

After thermal stress, the acetate buffer formulations purity ranged from 51.3% to 56.4%, the acidic species from 5.9% to 7.3%, and basic species from 37.2% to 41.8% (Table 11 above). The main peak decreased with decreasing pH, and the main peak percentage was also dependent on the excipient type. At pH 5.0, the main peak percentage was 54.0%, 55.2%, and 52.1% for sorbitol, trehalose, and sodium chloride respectively. No trend was observed for the amount of basic species observed under thermal stress. Under freeze/thaw stress in sodium acetate buffers, a similar trend was observed with pH and excipient relative to the thermal stress, although the difference between formulations are relatively small (Table 12 above). The IEX-HPLC results for the off-design formulations are found in Table 13 and Table 14.

TABLE 13

| | | | | Acid | Main | Basic | Main |
|---|---|---|---|---|---|---|---|
| | | | | Species | Peak | Species | Peak |
| Buffer | PH | Excipient | Condition | (%) | (%) | (%) | (RT) |
| 20 mM | 8.5 | 250 mM | 40° C., 2 W | 13.2 | 57.8 | 29.0 | 19.2 |
| Tris | | Trehalose | | | | | |
| | | 250 mM | | 14.2 | 57.5 | 28.3 | 19.2 |
| | | Sorbitol | | | | | |
| | | 250 mM | | 13.4 | 61.6 | 24.9 | 18.6 |
| | | NaCl | | | | | |

TABLE 14

| | | | | Acid | Mean | Basic | Main |
|---|---|---|---|---|---|---|---|
| | | | Sur- | Species | Peak | Species | Peak |
| Buffer | PH | Excipient | factant | (%) | (%) | (%) | (RT) |
| 20 mM | 8.5 | 250 mM | — | 7.5 | 69.6 | 22.8 | 19.2 |
| Tris | | Trehalose | | | | | |
| | | 250 mM | | 7.0 | 70.1 | 22.9 | 19.2 |
| | | Sorbitol | | | | | |
| | | 250 mM | | 7.8 | 69.7 | 22.5 | 19.4 |
| | | NaCl | | | | | |
| 20 mM | 7.5 | 250 mM | 0.02% | 9.2 | 68.1 | 22.7 | 19.4 |
| Phosphate | | Sorbitol | PS-80 | | | | |
| | | 250 | | 8.6 | 68.8 | 22.6 | 19.4 |
| | | Trehalose | | | | | |
| 20 mM | 5.25 | 250 mM | | 6.2 | 69.6 | 24.2 | 19.5 |
| Acetate | | Sorbitol | | | | | |
| | | 250 | | 6.2 | 69.3 | 24.5 | 19.5 |
| | | Trehalose | | | | | |

All formulations were colorless and clear after vialing. After freeze/thaw stress all formulations were colorless and slightly opalescent (Table 15, Table 16, and Table 17). Under thermal stress for the sodium phosphate formulations, product related precipitate was observed at pH 8.0 for sorbitol, pH 7.5 and 8.0 for trehalose, and in all sodium chloride formulations. For the sodium acetate formulations, particulate was observed in all formulations except 250 mM sorbitol at pH 4.5 and 250 mM trehalose at pH 5.5.

TABLE 15

| | | Freeze/Thaw | | | 40° C./75% RH | | |
|---|---|---|---|---|---|---|---|
| pH | Excipient | Color | Clarity | Particles | Color | Clarity | Particles |
| 7.0 | 250 mM | Colorless | Slightly | No Visible | Colorless | Slightly | No Visible |
| | Sorbitol | | Opalescent | Particulates | | Opalescent | Particulates |
| | | Colorless | Slightly | No Visible | Colorless | Slightly | No Visible |
| | | | Opalescent | Particulates | | Opalescent | Particulates |
| 7.5 | | Colorless | Slightly | No Visible | Colorless | Slightly | No Visible |
| | | | Opalescent | Particulates | | Opalescent | Particulates |
| 8.0 | | Colorless | Slightly | No Visible | Colorless | Slightly | No Visible |
| | | | Opalescent | Particulates | | Opalescent | Particulates |
| | | Colorless | Slightly | No Visible | Colorless | Slightly | YES |
| | | | Opalescent | Particulates | | Opalescent | |
| 7.0 | 250 mM | Colorless | Slightly | No Visible | Colorless | Slightly | No Visible |
| | Trehalose | | Opalescent | Particulates | | Opalescent | Particulates |
| | | Colorless | Slightly | No Visible | Colorless | Slightly | No Visible |
| | | | Opalescent | Particulates | | Opalescent | Particulates |
| 7.5 | | Colorless | Slightly | No Visible | Colorless | Slightly | YES |
| | | | Opalescent | Particulates | | Opalescent | |
| 8.0 | | Colorless | Slightly | No Visible | Colorless | Slightly | YES |
| | | | Opalescent | Particulates | | Opalescent | |
| | | Colorless | Slightly | No Visible | Colorless | Slightly | YES |
| | | | Opalescent | Particulates | | Opalescent | |
| 7.0 | 150 mM | Colorless | Slightly | No Visible | Colorless | Slightly | YES |
| | NaCl | | Opalescent | Particulates | | Opalescent | |
| | | Colorless | Slightly | No Visible | Colorless | Slightly | YES |
| | | | Opalescent | Particulates | | Opalescent | |
| 7.5 | | Colorless | Slightly | No Visible | Colorless | Partly | YES |
| | | | Opalescent | Particulates | | Cloudy | |
| 8.0 | | Colorless | Slightly | No Visible | Colorless | Partly | YES |
| | | | Opalescent | Particulates | | Cloudy | |
| | | Colorless | Slightly | No Visible | Colorless | Partly | YES |
| | | | Opalescent | Particulates | | Cloudy | |

TABLE 16

| | | Freeze/Thaw | | | 40° C./75% RH | | |
|---|---|---|---|---|---|---|---|
| pH | Excipient | Color | Clarity | Particles | Color | Clarity | Particles |
| 4.5 | 250 mM | Colorless | Slightly | No Visible | Colorless | Slightly | No Visible |
| | Sorbitol | | Opalescent | Particulates | | Opalescent | Particulates |
| | | Colorless | Slightly | No Visible | Colorless | Slightly | No Visible |
| | | | Opalescent | Particulates | | Opalescent | Particulates |
| 5.0 | | Colorless | Slightly | No Visible | Colorless | Slightly | YES |
| | | | Opalescent | Particulates | | Opalescent | |
| 5.5 | | Colorless | Slightly | No Visible | Colorless | Slightly | YES |
| | | | Opalescent | Particulates | | Opalescent | |
| | | Colorless | Slightly | No Visible | Colorless | Slightly | YES |
| | | | Opalescent | Particulates | | Opalescent | |
| 4.5 | 250 mM | Colorless | Slightly | No Visible | Colorless | Slightly | YES |
| | Trehalose | | Opalescent | Particulates | | Opalescent | |
| | | Colorless | Slightly | No Visible | Colorless | Slightly | No Visible |
| | | | Opalescent | Particulates | | Opalescent | Particulates |
| 5.0 | | Colorless | Slightly | No Visible | Colorless | Slightly | YES |
| | | | Opalescent | Particulates | | Opalescent | |
| 5.5 | | Colorless | Slightly | No Visible | Colorless | Slightly | No Visible |
| | | | Opalescent | Particulates | | Opalescent | Particulates |
| | | Colorless | Slightly | No Visible | Colorless | Slightly | No Visible |
| | | | Opalescent | Particulates | | Opalescent | Particulates |
| 4.5 | 150 mM | Colorless | Slightly | No Visible | Colorless | Cloudy | Precipitate |
| | NaCl | | Opalescent | Particulates | | | |
| | | Colorless | Slightly | No Visible | Colorless | Cloudy | Precipitate |
| | | | Opalescent | Particulates | | | |
| 5.0 | | Colorless | Slightly | No Visible | Colorless | Partly Cloudy | Precipitate |
| | | | Opalescent | Particulates | | | |
| 5.5 | | Colorless | Slightly | No Visible | Colorless | Slightly | YES |
| | | | Opalescent | Particulates | | Opalescent | |
| | | Colorless | Slightly | No Visible | Colorless | Slightly | YES |
| | | | Opalescent | Particulates | | Opalescent | |

TABLE 17

| | | | | Freeze/Thaw | | | 40° C./75% RH | | |
|---|---|---|---|---|---|---|---|---|---|
| Buffer | pH | Excipient | Surfactant | Color | Clarity | Particles | Colar | Clarity | Particles |
| 20 mM | 8.5 | 250 mM | — | Colorless | Slightly | No Visible | Colorless | Partly | No Visible |
| Tris | | Trehalose | | | Opalescent | Particulates | | Cloudy | Particulates |
| | | 250 mM | | Colorless | Slightly | No Visible | Colorless | Partly | No Visible |
| | | Sorbitol | | | Opalescent | Particulates | | Cloudy | Particulates |
| | | 150 mM | | Colorless | Slightly | No Visible | Colorless | Partly | YES |
| | | NaCl | | | Opalescent | Particulates | | Cloudy | |
| 20 mM | 7.5 | 250 mM | 0.02% | Colorless | Slightly | No Visible | | | |
| Phosphate | | Sorbitol | PS-80 | | Opalescent | Particulates | | | |
| | | 250 mM | | Coloriess | Slightly | No Visible | | | |
| | | Trehalose | | | Opalescent | Particulates | | | |
| 20 mM | 5.3 | 250 mM | | Colorless | Slightly | No Visible | | | |
| Acetate | | Sorbitol | | | Opalescent | Particulates | | | |
| | | 250 mM | | Colorless | Slightly | No Visible | | | |
| | | Trehalose | | | Opalescent | Particulates | | | |

The analytical data for the sodium phosphate buffered formulations under thermal stress was selected for further analysis due to the degree of difference between the formulations, and the client's historical experience with RC in phosphate buffers. The results from the SEC, IEX, and appearance testing showed a clear trend on pH for formulation stability, with the excipient showing a lesser effect for the formulations at pH 7.0.

The numerical optimization was performed for the sodium phosphate formulations under thermal stress using Stat-Ease, Inc. Design-Expert® Version 9.0.6.2 in order to maximize the SEC and IEX main peak percentages and minimize the formation of LMW and HMW species in SEC and acidic and basic species in IEX. The numerical optimization was performed on the analytical results from the 20 mM sodium phosphate DOE after 2 weeks of thermal stress. The formulation optimization was performed to find the optimal formulation without any constraints on the pH or excipient. The optimization parameters and results are found in Table 18, Table 19, Table 20, and Table 21. When the presence of particulate in the formulations used as a response and all results were given equal importance, the optimal formulation was calculated to be 20 mM sodium phosphate, 250 mM trehalose at pH 7.2. If the appearance of particulate in the formulation is not factor as a constraint in the optimization and all results were given the same importance, the optimal formulation was calculated to be 20 mM sodium phosphate, 150 mM sodium chloride at pH 7.0. Based on the DOE and off-DOE formulations, the 20 mM sodium phosphate, 250 mM trehalose, pH 7.0 at 20 mg/mL RC was selected for further testing in the surfactant study.

TABLE 18

| | | Lower | Upper | Lower | Upper | |
|---|---|---|---|---|---|---|
| Name | Goal | Limit | Limit | Weight | Weight | Importance |
| A: pH | is in range | 7 | 8 | 1 | 1 | 3 |
| B: Excipient | is in range | Sorbitol | NaCl | 1 | 1 | 3 |
| SEC (HMW) | minimize | 0.66 | 1 | 1 | 1 | 3 |
| SEC (Main) | maximize | 88.29 | 99.31 | 1 | 1 | 3 |
| SEC (LMW) | minimize | 0 | 10.98 | 1 | 1 | 3 |
| IEX acidic % | minimize | 9.2587 | 18.7732 | 1 | 1 | 3 |
| IEX % Main Peak | maximize | 52.9391 | 63.1144 | 1 | 1 | 3 |
| IEX Basic % | minimize | 25.2444 | 30.0589 | 1 | 1 | 3 |

TABLE 19

| | | | | | | | IEX % | IEX | |
|---|---|---|---|---|---|---|---|---|---|
| | | | SEC % | SEC % | SEC % | IEX % | Main | Basic | Desira- |
| Number | pH | Excipient | HMW | Main | LMW | Acidic | Peak | % | bility |
| 1 | 7.0 | NaCl | 0.705 | 99.167 | 0.016 | 9.25 | 62.55 | 28.07 | 0.833 |
| 2 | 7.3 | Trehalose | 0.788 | 96.394 | 2.788 | 12.20 | 58.57 | 29.32 | 0.521 |

TABLE 20

| | | Lower | Upper | Lower | Upper | |
|---|---|---|---|---|---|---|
| Name | Goal | Limit | Limit | Weight | Weight | Importance |
| A: pH | is in range | 7 | 8 | 1 | 1 | 3 |
| B: Excipient | is in range | Sorbitol | NaCl | 1 | 1 | 3 |
| SEC (HMW) | minimize | 0.66 | 1 | 1 | 1 | 3 |
| SEC (Main) | maximize | 88.29 | 99.31 | 1 | 1 | 3 |
| SEC (LMW) | minimize | 0 | 10.98 | 1 | 1 | 3 |
| IEX acidic % | minimize | 9.2587 | 18.7732 | 1 | 1 | 3 |
| IEX % Main Peak | maximize | 52.9391 | 63.1144 | 1 | 1 | 3 |
| IEX Basic % | minimize | 25.2444 | 30.0589 | 1 | 1 | 3 |
| Particulate | minimize | 0 | 1 | 1 | 1 | 3 |

TABLE 21

| | | | | | | | IEX % | | | |
|---|---|---|---|---|---|---|---|---|---|---|
| | | | SEC % | SEC % | SEC % | IEX % | Main | IEX % | | |
| Number | pH | Excipient | HMW | Main | LMW | Acidic | Peak | Basic | Particulate | Desirability |
| 1 | 7.2 | Trehalose | 0.788 | 97.037 | 2.145 | 11.82 | 53.78 | 29.51 | 0.332 | 0.536 |
| 2 | 7.0 | Sorbitol | 0.959 | 99.167 | 0.016 | 13.16 | 57.44 | 29.31 | −0.050 | 0.467 |

### Example 3: Surfactant Study

A surfactant screening study was performed to evaluate the impact of PS-80 concentration on observed opalescence after freeze/thaw or agitation stress. L-asparaginase was formulated in 20 mM sodium phosphate at pH 6.5, 7.0, and 7.5 with 250 mM trehalose at PS-80 concentrations between 0.00% and 0.06% w/v (Table 22). As an alternative formulation, 20 mM sodium acetate, 250 mM Trehalose at pH 5.5 with 0.00% and 0.04% w/v PS-80 was included.

TABLE 22

| | | | | Time | Concentration |
|---|---|---|---|---|---|
| Buffer | Excipient | pH | PS-80 (%) | Point/Condition | (mg/mL) |
| 20 mM | 250 mM | 7.0 | 0.00% | T0 | 20 |
| Sodium | Trehalose | | | 1 F/T | 21 |
| Phosphate | | | | 3 F/T | 21 |
| | | | | 0 H Agitation | 21 |
| | | | | Control | |
| | | | | 24 H Agitation | 21 |
| | | | | 48 H Agitation | 21 |
| | | | 0.02% | T0 | 21 |
| | | | | 1 F/T | 21 |
| | | | | 3 F/T | 21 |
| | | | | 0 H Agitation | 21 |
| | | | | Control | |
| | | | | 24 H Agitation | 21 |
| | | | | 48 H Agitation | 21 |
| | | | 0.04% | T0 | 22 |
| | | | | 1 F/T | 22 |
| | | | | 3 F/T | 22 |
| | | | | 0 H Agitation | 22 |
| | | | | Control | |
| | | | | 24 H Agitation | 22 |
| | | | | 48 H Agitation | 22 |
| | | | 0.06% | T0 | 22 |
| | | | | 1 F/T | 21 |
| | | | | 3 F/T | 22 |
| | | | | 0 H Agitation | 22 |
| | | | | Control | |
| | | | | 24 H Agitation | 22 |
| | | | | 48 H Agitation | 22 |
| | | 6.5 | 0.04% | T0 | 20 |
| | | | | 1 F/T | 20 |
| | | | | 3 F/T | 20 |
| | | | | 0 H Agitation | 19 |
| | | | | Control | |
| | | | | 24 H Agitation | 20 |
| | | | | 48 H Agitation | 20 |
| | | 7.5 | | T0 | 21 |
| | | | | 1 F/T | 21 |
| | | | | 3 F/T | 21 |
| | | | | 0 H Agitation | 21 |
| | | | | Control | |
| | | | | 24 H Agitation | 21 |
| | | | | 48 H Agitation | 21 |
| 20 mM | 250 mM | 5.5 | 0.00% | T0 | 20 |
| Sodium | Trehalose | | | 1 F/T | 20 |
| Acetate | | | | 3 F/T | 20 |
| | | | | 0 H Agitation | 20 |
| | | | | Control | |
| | | | | 24 H Agitation | 20 |
| | | | | 48 H Agitation | 20 |
| | | | 0.04% | T0 | 21 |
| | | | | 1 F/T | 21 |
| | | | | 3 F/T | 21 |
| | | | | 0 H Agitation | 21 |
| | | | | Control | |
| | | | | 24 H Agitation | 21 |
| | | | | 48 H Agitation | 21 |

L-asparaginase (at 22 mg/mL, 1760 mg) was dialyzed into the corresponding formulation buffers without PS-80 using 10K MWCO Slide-A-Lyzer G2 dialysis cassettes. Dialysis was performed at room temperature with a minimum formulation buffer to sample volume of 40:1. Four dialysis buffer exchanges were performed. Following dialysis, the material was removed from the cassettes, and the concentration and pH were measured. The material was then split and PS-80 was added to the appropriate concentration using 10% (w/v) PS-80 solution in H₂O. After mixing, each of the formulations will be aliquoted equally into six 2 mL vials.

One vial from each formulation was held at 5° C. for the initial time point testing (8 total) during the freeze-thaw and agitation evaluation. One and three freeze-thaw cycles were performed independently on separate vials of each formulation. Each freeze-thaw cycle was frozen at −75±10° C. for a minimum of 2 hours and thawed at room temperature for a minimum of 2 hours and until completely thawed. Three vials from each formulation were required for the agitation evaluation (24 total). One vial was agitated on a rotator at 60 rpm at ambient temperature for 24 hours, one vial for 48 hours under the same conditions, and one vial was placed next to the rotator for the duration of the study as a control.

The surfactant study was performed to assess the impact on PS-80 on the development of opalescence under freeze/thaw or agitation stress for RC formulations in 20 mM phosphate, 250 mM trehalose at pH 6.5, 7.0, and 7.5 with 0.00%, 0.02%, 0.04%, and 0.06% PS-80 (Table 22). Two additional formulations were added to the study as potential buffer alternatives: 20 mM acetate, 250 mM trehalose at pH 5.5 with 0.00% and 0.04% PS-80. The formulations were then exposed to 1 and 3 freeze/thaw cycles at −75° C. and stressed with agitation for 24, and 48 hours at room temperature. Controls for each formulation were held at 5° C. for the freeze/thaw stress and room temperature for 48 hours without agitation.

By appearance testing, all control and stressed samples were colorless and slightly opalescence without product related particulate. There was no observed difference in the degree of opalescence between the control samples and the freeze/thaw and agitation samples or the different levels of PS-80. The protein concentration was also assessed for controls and stressed formulations. The protein concentration was between 19 and 22 mg/ml (Table 22). No significant changes or trends were observed upon freeze/thaw or agitation stress.

The DLS analysis showed that all samples had a hydrodynamic radius between 4.1 and 4.5 with a percent polydispersity between 10.7% and 19.4% (Table 23 and Table 24). No significant differences or trends were observed in the DLS relative to formulation or stress condition.

TABLE 23

| | PS-80 | Time | Radius (nm) | % Pd |
|---|---|---|---|---|
| pH | (%) | Point/Condition | Average | Average |
| 7.0 | 0.00% | T0 | 4.2 | 15.5 |
| | | 1 F/T | 4.3 | 18.4 |
| | | 3 F/T | 4.3 | 16.7 |
| | | 48 H Agitation | 4.4 | 17.6 |
| | | Control | | |
| | | 24 H Agitation | 4.3 | 14.8 |
| | | 48 H Agitation | 4.4 | 16.6 |
| | 0.02% | T0 | 4.4 | 15.4 |
| | | 1 F/T | 4.4 | 15.6 |
| | | 3 F/T | 4.3 | 14.4 |
| | | 48 H Agitation | 4.5 | 19.4 |
| | | Control | | |
| | | 24 H Agitation | 4.4 | 16.7 |
| | | 48 H Agitation | 4.4 | 14.5 |
| 7.0 | 0.04% | T0 | 4.5 | 16.3 |
| | | 1 F/T | 4.3 | 12.2 |
| | | 3 F/T | 4.3 | 16.3 |
| | | 48 H Agitation | 4.4 | 17.2 |
| | | Control | | |
| | | 24 H Agitation | 4.4 | 15.4 |
| | | 48 H Agitation | 4.3 | 14.7 |
| | 0.06% | T0 | 4.4 | 16.6 |
| | | 1 F/T | 4.4 | 14.2 |
| | | 3 F/T | 4.4 | 17.3 |
| | | 48 H Agitation | 4.4 | 14.9 |
| | | Control | | |
| | | 24 H Agitation | 4.4 | 14.0 |
| | | 48 H Agitation | 4.5 | 17.3 |
| 6.5 | 0.04% | T0 | 4.3 | 16.1 |
| | | 1 F/T | 4.2 | 11.6 |
| | | 3 F/T | 4.3 | 13.1 |
| | | 48 H Agitation | 4.3 | 10.7 |
| | | Control | | |
| | | 24 H Agitation | 4.3 | 13.7 |
| | | 48 H Agitation | 4.3 | 12.9 |
| 7.5 | | T0 | 4.2 | 17.1 |
| | | 1 F/T | 4.3 | 15.7 |
| | | 3 F/T | 4.3 | 13.6 |
| | | 48 H Agitation | 4.3 | 14.3 |
| | | Control | | |
| | | 24 H Agitation | 4.3 | 12.6 |
| | | 48 H Agitation | 4.3 | 14.6 |

TABLE 24

| | PS-80 | Time | Radius (nm) | % Pd |
|---|---|---|---|---|
| pH | (%) | Point/Condition | Average | Average |
| 5.5 | 0.00% | T0 | 4.3 | 14.6 |
| | | 1 F/T | 4.4 | 14.2 |
| | | 3 F/T | 4.3 | 14.0 |
| | | 48 H Agitation | 4.1 | 14.4 |
| | | Control | | |
| | | 24 H Agitation | 4.1 | 15.6 |
| | | 48 H Agitation | 4.2 | 15.2 |
| | 0.04% | T0 | 4.3 | 16.6 |
| | | 1 F/T | 4.3 | 15.0 |
| | | 3 F/T | 4.4 | 15.5 |
| | | 48 H Agitation | 4.3 | 16.3 |
| | | Control | | |
| | | 24 H Agitation | 4.4 | 16.5 |
| | | 48 H Agitation | 4.4 | 16.3 |

The SEC-HPLC results show that all formulations, regardless of stress, in 20 mM phosphate buffer had similar profiles with the main peak percentage of 99.3% and a HMW peak of 0.7% before and after stress conditions (Table 25). Similarly, the SECHPLC of the 20 mM acetate formulations showed 0.7% HMW peak, between 97.9% to 98.1% main peak, and between 1.3% to 1.4% LMW peak (Table 26).

TABLE 25

| | PS-80 | | HMW Peak | RC Peak | LMW Peak |
|---|---|---|---|---|---|
| pH | (%) | Condition | (%) | (%) | (%) |
| 7.0 | 0.00% | T0 | 0.71 | 99.29 | ND |
| | | 1 F/T | 0.70 | 99.30 | ND |
| | | 3 F/T | 0.70 | 99.30 | ND |
| | | 48 H Agitation | 0.69 | 99.31 | ND |
| | | Control | | | |
| | | 24 H Agitation | 0.71 | 99.29 | ND |
| | | 48 H Agitation | 0.71 | 99.29 | ND |
| | 0.02% | T0 | 0.72 | 99.28 | ND |
| | | 1 F/T | 0.71 | 99.29 | ND |
| | | 3 F/T | 0.72 | 99.28 | ND |
| | | 48 H Agitation | 0.72 | 99.29 | ND |
| | | Control | | | |
| | | 24 H Agitation | 0.72 | 99.28 | ND |
| | | 48 H Agitation | 0.72 | 99.28 | ND |
| 7.0 | 0.04% | T0 | 0.71 | 99.29 | ND |
| | | 1 F/T | 0.71 | 99.29 | ND |
| | | 3 F/T | 0.71 | 99.29 | ND |
| | | 48 H Agitation | 0.71 | 99.29 | ND |
| | | Control | | | |
| | | 24 H Agitation | 0.70 | 99.30 | ND |
| | | 48 H Agitation | 0.71 | 99.29 | ND |
| | 0.06% | T0 | 0.72 | 99.28 | ND |
| | | 1 F/T | 0.72 | 99.28 | ND |
| | | 3 F/T | 0.73 | 99.27 | ND |
| | | 48 H Agitation | 0.71 | 99.29 | ND |
| | | Control | | | |
| | | 24 H Agitation | 0.70 | 99.30 | ND |
| | | 48 H Agitation | 0.72 | 99.28 | ND |
| 6.5 | 0.04% | T0 | 0.70 | 99.30 | ND |
| | | 1 F/T | 0.66 | 99.34 | ND |
| | | 3 F/T | 0.71 | 99.29 | ND |
| | | 48 H Agitation | 0.70 | 99.30 | ND |
| | | Control | | | |
| | | 24 H Agitation | 0.70 | 99.30 | ND |
| | | 48 H Agitation | 0.70 | 99.30 | ND |
| 7.5 | | T0 | 0.73 | 99.27 | ND |
| | | 1 F/T | 0.73 | 99.27 | ND |
| | | 3 F/T | 0.71 | 99.29 | ND |
| | | 48 H Agitation | 0.72 | 99.28 | ND |
| | | Control | | | |
| | | 24 H Agitation | 0.73 | 99.27 | ND |
| | | 48 H Agitation | 0.72 | 99.28 | ND |

TABLE 26

| | PS-80 | | HMW Peak | RC Peak | LMW Peak |
|---|---|---|---|---|---|
| pH | (%) | Condition | (%) | (%) | (%) |
| 5.5 | 0.00% | T0 | 0.68 | 97.95 | 1.37 |
| | | 1 F/T | 0.68 | 98.00 | 1.33 |
| | | 3 F/T | 0.69 | 98.01 | 1.30 |
| | | 48 H Agitation | 0.67 | 98.01 | 1.32 |
| | | Control | | | |
| | | 24 H Agitation | 0.66 | 98.06 | 1.27 |
| | | 48 H Agitation | 0.67 | 98.00 | 1.34 |
| | 0.04% | T0 | 0.68 | 98.03 | 1.29 |
| | | 1 F/T | 0.68 | 97.92 | 1.40 |
| | | 3 F/T | 0.70 | 97.94 | 1.36 |
| | | 48 H Agitation | 0.68 | 98.00 | 1.32 |
| | | Control | | | |
| | | 24 H Agitation | 0.68 | 97.99 | 1.33 |
| | | 48 H Agitation | 0.69 | 97.97 | 1.34 |

Sub-visible particulates were measured by HIAC for the T0, 1 freeze/thaw cycle, 3 freeze/thaw cycles, agitation control, 24 hour agitation, and 48 hour agitation samples with varying concentrations of PS-80. The results are summarized in Table 27 and Table 28. For all samples the cumulative counts/mL ranged from 2330-45270, 390-35857, 78-6430, and 0-150 for ≥2 μm, 5 μm, ≥10 μm, ≥25 μm, respectively. A few conditions had elevated particulate levels relative to the other conditions of the same formulation, but these samples appear to be outliers rather than representing a trend. For example, 20 mM phosphate, 250 mM trehalose, 0.04% PS-80, pH 7.0 solution had 2842≥10 μm particles at the initial timepoint, but only 155 particles in the agitation control.

TABLE 27

| | PS-80 | Time | Cumulative Counts/mL | | | |
|---|---|---|---|---|---|---|
| pH | (%) | Point/Condition | ≥2 μm | ≥5 μm | ≥10 μm | ≥25 μm |
| 7.0 | 0.00% | T0 | 15613 | 2656 | 522 | 40 |
| | | 1 F/T¹ | 45270 | 21660 | 6430 | 150 |
| | | 3 F/T | 8538 | 1447 | 313 | 8 |
| | | 0 H Agitation Control | 18195 | 3640 | 765 | 37 |
| | | 24 H Agitation | 5148 | 865 | 167 | 2 |
| | | 48 H Agitation | 18402 | 3172 | 578 | 23 |
| | 0.02% | T0 | 43832 | 16540 | 3573 | 52 |
| | | 1 F/T | 4735 | 877 | 170 | 3 |
| | | 3 F/T | 5857 | 1027 | 188 | 12 |
| | | 0 H Agitation Control | 3028 | 490 | 105 | 0 |
| | | 24 H Agitation | 4350 | 815 | 152 | 2 |
| | | 48 H Agitation | 17782 | 3280 | 498 | 8 |
| | 0.04% | T0 | 41718 | 14653 | 2842 | 38 |
| | | 1 F/T | 4887 | 883 | 173 | 5 |
| | | 3 F/T | 5700 | 1080 | 252 | 10 |
| | | 0 H Agitation Control | 3123 | 633 | 155 | 10 |
| | | 24 H Agitation | 9640 | 2122 | 457 | 13 |
| | | 48 H Agitation | 18538 | 35857 | 680 | 3 |
| | 0.06% | T0 | 16930 | 2632 | 373 | 7 |
| | | 1 FIT | 16795 | 4673 | 1037 | 17 |
| | | 3 F/T | 6238 | 1172 | 258 | 5 |
| | | 0 H Agitation Control | 7500 | 1602 | 327 | 8 |
| | | 24 H Agitation | 4250 | 828 | 163 | 2 |
| | | 48 H Agitation | 20525 | 4120 | 693 | 7 |
| 8.5 | 0.04% | T0 | 14865 | 1873 | 170 | 3 |
| | | 1 FIT | 4748 | 768 | 120 | 3 |
| | | 3 F/T | 2330 | 390 | 78 | 0 |
| | | 0 H Agitation Control | 6888 | 963 | 168 | 10 |
| | | 24 H Agitation | 9677 | 2383 | 495 | 5 |
| | | 48 H Agitation | 11253 | 1483 | 190 | 7 |
| 7.5 | | T0 | 30553 | 8140 | 1243 | 13 |
| | | 1 F/T | 7458 | 1168 | 185 | 5 |
| | | 3 F/T | 3843 | 643 | 132 | 2 |
| | | 0 H Agitation Control | 2640 | 497 | 105 | 2 |
| | | 24 H Agitation | 15910 | 2568 | 448 | 17 |
| | | 48 H Agitation | 19282 | 3733 | 622 | 10 |

¹The sample testing was interrupted and did not fully complete. However, the measurement(s) taken represent actual result for sample

TABLE 28

| | PS-80 | Time | | | | |
|---|---|---|---|---|---|---|
| pH | (%) | Point/Condition | ≥2 μm | ≥5 μm | ≥10 μm | ≥25 μm |
| 5.5 | 0.00% | T0 | 11538 | 1735 | 242 | 10 |
| | | 1 F/T | 6808 | 1260 | 218 | 10 |
| | | 3 F/T | 14612 | 2465 | 418 | 22 |
| | | 0 H | 8667 | 1432 | 353 | 23 |
| | | Agitation | | | | |
| | | Control | | | | |
| | | 24 H | 11217 | 2040 | 353 | 23 |
| | | Agitation | | | | |
| | | 48 H | 16168 | 3152 | 517 | 20 |
| | | Agitation | | | | |
| | 0.04% | T0¹ | 12550 | 1830 | 233 | 8 |
| | | 1 F/T | 6282 | 1172 | 148 | 5 |
| | | 3 F/T | 12148 | 2190 | 423 | 13 |
| | | 0 H | 2955 | 600 | 110 | 2 |
| | | Agitation | | | | |
| | | Control | | | | |
| | | 24 H | 10077 | 1700 | 215 | 0 |
| | | Agitation | | | | |
| | | 48 H | 14297 | 2437 | 315 | 3 |
| | | Agitation | | | | |

IEX-HPLC was performed for all the 20 mM phosphate formulations at all conditions. The main peak percentage for the TO control at pH 7.0 with 0%, 0.02%, 0.04%, and 0.06% PS-80 was 68.88%, 69.83%, 71.36%, and 70.38%, respectively (Table 29). At 0.04% PS-80, the main peak percentage for the TO control at pH 6.5, 7.0, and 7.5 was 70.11%, 71.36%, and 68.04%, respectively. No significant change in the IEX profile of all formulations was observed after exposure to freeze/thaw or agitation stress. These results indicate little benefit in the addition of PS-80 up to 0.06% w/v.

TABLE 29

| | | | Acidic | | Main Peak | Basic |
|---|---|---|---|---|---|---|
| | PS-80 | | Species | Main Peak | Purity | Species |
| pH | (%) | Condition | (%) | (RT) | (%) | (%) |
| 7.0 | 0.00% | T0 | 11.78 | 19.15 | 68.88 | 19.34 |
| | | 1 F/T | 10.35 | 19.14 | 68.98 | 20.67 |
| | | 3 F/T | 10.01 | 19.13 | 69.24 | 20.75 |
| | | 48 H Control | 10.12 | 19.10 | 68.72 | 21.17 |
| | | 24 H Agitation | 9.47 | 19.10 | 69.26 | 21.27 |
| | | 48 H Agitation | 9.44 | 19.08 | 69.26 | 21.30 |
| | 0.02% | T0 | 9.06 | 19.08 | 69.83 | 21.12 |
| | | 1 F/T | 8.77 | 19.07 | 70.44 | 20.78 |
| | | 3 F/T | 8.38 | 19.07 | 70.66 | 20.96 |
| | | 48 H Control | 9.04 | 19.07 | 69.87 | 21.09 |
| | | 24 H Agitation | 9.35 | 19.07 | 69.73 | 20.92 |
| | | 48 H Agitation | 9.13 | 19.08 | 69.85 | 21.02 |
| | 0.04% | T0 | 8.89 | 19.08 | 71.36 | 19.75 |
| | | 1 F/T | 8.97 | 19.07 | 70.67 | 20.36 |
| | | 3 F/T | 8.98 | 19.07 | 70.94 | 20.08 |
| | | 48 H Control | 9.23 | 19.07 | 70.87 | 19.90 |
| | | 24 H Agitation | 8.93 | 19.08 | 71.24 | 18.84 |
| | | 48 H Agitation | 9.38 | 19.08 | 70.52 | 20.09 |
| | 0.06% | T0 | 8.87 | 19.08 | 70.38 | 20.75 |
| | | 1 F/T | 9.20 | 19.09 | 70.65 | 20.15 |
| | | 3 F/T | 9.88 | 19.08 | 69.80 | 20.32 |
| | | 48 H Control | 9.55 | 19.08 | 69.41 | 21.04 |
| | | 24 H Agitation | 9.57 | 19.08 | 68.52 | 20.91 |
| | | 48 H Agitation | 9.88 | 19.08 | 69.01 | 21.12 |
| 6.5 | 0.04% | T0 | 7.15 | 19.08 | 70.11 | 20.84 |
| | | 1 F/T | 8.98 | 19.08 | 70.16 | 20.86 |
| | | 3 F/T | 9.28 | 19.09 | 69.64 | 21.07 |
| | | 48 H Control | 9.50 | 19.09 | 69.25 | 21.25 |
| | | 24 H Agitation | 9.23 | 19.08 | 69.67 | 21.11 |
| | | 48 H Agitation | 9.78 | 19.08 | 68.92 | 21.30 |
| 7.5 | | T0 | 11.32 | 19.08 | 68.04 | 20.64 |
| | | 1 F/T | 10.78 | 19.08 | 68.65 | 20.56 |
| | | 3 F/T | 10.70 | 19.08 | 68.79 | 20.51 |
| | | 48 H Control | 10.91 | 19.09 | 68.07 | 21.02 |
| | | 24 H Agitation | 10.57 | 19.08 | 69.05 | 20.38 |
| | | 48 H Agitation | 10.82 | 19.08 | 68.18 | 21.00 |

### Example 4: Salt/Trehalose Dependence Study

The preformation salt/trehalose dependence study performed at 22 mg/mL L-asparaginase was formulated in nine different formulation buffers, according to Table 30. An aliquot of 27 mL of Lot RE-LAP-P59 was divided into 6 portions of approximately 3 mL (3 portions) and 6 mL (3 portions). A buffer exchange was performed for formulations 1-6 via dialysis using 10K MWCO Slide-A-Lyzer G2 Cassettes. Dialysis was performed at room temperature at a minimum buffer to sample volume ratio of 40:1. Four dialysis buffer exchanges were performed for each formulation approximately every 2 hours, with one exchange occurring overnight.

TABLE 30

| | | | | | Lot RE-LAP-P59 |
|---|---|---|---|---|---|
| | | Trehalose | NaCl | PS-80% | Volume to |
| Formulation | Buffer | (mM) | (mM) | (w/v) | be Dialyzed |
| F1 | 20 mM Na | 175 | 0 | — | 6 mL |
| F2 | Phosphate | | 50 | | 6 mL |
| F3 | | | 100 | | 6 mL |
| F4 | 50 mM Na | | 0 | | 3 mL |
| F5 | Phosphate | | 50 | | 3 mL |
| F6 | | | 100 | | 3 mL |
| F7 | 20 mM Na | | 0 | 0.02 | No Dialysis |
| F8 | Phosphate | | 50 | | No Dialysis |
| F9 | | | 100 | | No Dialysis |

Following dialysis, the material was removed from the dialysis cassettes and the pH and protein concentration were confirmed. The concentration of the samples was then adjusted to 20±2 mg/mL with the corresponding buffer. After adjustment, the material in buffers 1-3 was split into equal portions of 3 mL each. One set of these aliquots was supplemented with 10% PS-80 to a final concentration of 0.02%. All samples were then filtered and vialed in a biosafety cabinet. One set of the formulations were stored at 5° ° C. until testing for the initial material quality evaluation, one set was stored at 5° C. for 3 weeks, and one set was subjected to a heat stress at 40° C. for 3 weeks. The 3 week time point was selected after an evaluation of the visual appearance for after 1.5 weeks storage at 40° C. Formulations one and two at 5° C. were additionally tested by IEX and SEC HPLC after seven weeks.

The salt/trehalose dependence study was performed to assess the impact of sodium chloride concentration on L-asparaginase formulations in 20 mM or 50 mM phosphate, 175 mM trehalose, at pH 7.0 with and without 0.02% PS-80 (Table 30). The formulations were exposed to 1.5 and 3 weeks at 5° ° C. and 40° C. Size exclusion and IEX chromatography were used to assess the stability of RC in the presence of these varying salt concentrations in the selected buffers. Osmolality and conductivity were also determined at the initial timepoint and are shown in Table 31.

TABLE 31

| | Excipient | Excipient | PS-80% | | Osmolality | Conductivity |
|---|---|---|---|---|---|---|
| Buffer | 1 | 2 | (w/v) | pH | (mOsm/kg) | (mS/cm) |
| 20 mM Na | 175 mM | — | — | 7.0 | 240 | 2.3 |
| Phosphate | Trehalose | 50 mM NaCl | — | | 336 | 7.0 |
| | | 100 mM NaCl | — | | 428 | 11.7 |
| 50 mM Na | | — | — | | 305 | 5.3 |
| Phosphate | | 50 mM NaCl | — | | 401 | 10.0 |
| | | 100 mM NaCl | — | | 498 | 14.5 |
| 20 mM Na | | — | 0.02 | | 242 | 2.4 |
| Phosphate | | 50 mM NaCl | 0.02 | | 336 | 7.1 |
| | | 100 mM NaCl | 0.02 | | 431 | 11.8 |

By appearance testing, all initial and stressed samples were colorless and slightly opalescence with very-few to no product related particulates. All formulations became more opalescent as a function of stress. The 0.02% PS-80 containing formulations did not have any particulates present regardless of storage duration (Table 32). This result indicates that addition of 0.02% PS-80 may delay particle formulation during long term storage. There was no dependence of appearance on salt concentration. The protein concentration was also assessed for initial and stressed formulations. The protein concentration was between 19.7 and 25.2 mg/mL (Table 33). No significant changes or trends were observed in the stressed samples.

TABLE 32

| | | | PS- | | | | | | |
|---|---|---|---|---|---|---|---|---|---|
| | Excipient | Excipient | 80% | | Time | | | | |
| Buffer | 1 | 2 | (w/v) | pH | Point | Condition | Color | Clarity | Particulates |
| 20 mM Na | 175 mM | — | — | 7.0 | T0 | N/A | Colorless Liquid | Clear Liquid | Free of visible |
| Phosphate | Trehalose | | | | | | | | Particulates |
| | | | | | 1.5 W | 40° C. | Colorless Liquid | Opalescence is not more | Free of visible |
| | | | | | | | | pronounce than | Particulates |
| | | | | | | | | Reference Suspension IV | |
| | | | | | 3 W | 5° C. | Colorless Liquid | Opalescence is not more | Free of visible |
| | | | | | | | | pronounce than | Particulates |
| | | | | | | | | Reference Suspension IV | |
| | | | | | | 40° C. | Colorless Liquid | Opalescence is not more | Very Few |
| | | | | | | | | pronounce than | Particulates |
| | | | | | | | | Reference Suspension IV | |
| | | 50 mM | — | | T0 | N/A | Colorless Liquid | Clear Liquid | Free of visible |
| | | Nacl | | | | | | | Particulates |
| | | | | | 1.5 W | 40° C. | Colorless Liquid | Opalescence is not more | Free of visible |
| | | | | | | | | pronounce than | Particulates |
| | | | | | | | | Reference Suspension IV | |
| | | | | | 3 W | 5° C. | Colorless Liquid | Opalescence is not more | Free of visible |
| | | | | | | | | pronounce than | Particulates |
| | | | | | | | | Reference Suspension IV | |
| | | | | | | 40° C. | Colorless Liquid | Opalescence is not more | Very Few |
| | | | | | | | | pronounce than | Particulates |
| | | | | | | | | Reference Suspension IV | |
| | | 100 mM | — | | T0 | N/A | Colorless Liquid | Clear Liquid | Free of visible |
| | | NaCl | | | | | | | Particulates |
| | | | | | 1 W | 40° C. | Colorless Liquid | Opalescence is not more | Free of visible |
| | | | | | | | | pronounce than | Particulsies |
| | | | | | | | | Reference Suspension IV | |
| | | | | | 1.5 W | | Colorless Liquid | Opalescence is not more | Free of visible |
| | | | | | | | | pronounce than | Particulates |
| | | | | | | | | Reference Suspension IV | |
| | | | | | 3 W | 40° C. | Colorless Liquid | Opalescence is not more | Very Few |
| | | | | | | | | pronounce than | Particulates |
| | | | | | | | | Reference Suspension IV | |
| 50 mM Na | | — | — | | T0 | N/A | Colorless Liquid | Clear Liquid | Free of visittle |
| Phosphate | | | | | | | | | Particulates |
| | | | | | 1.5 W | 40° C. | Colorless Liquid | Opalescence is not more | Free of visible |
| | | | | | | | | pronounce than | Particulstes |
| | | | | | | | | Reference Suspension III | |
| | | | | | 3 W | 40° C. | Colorless Liquid | Opalescence is not more | Very Few |
| | | | | | | | | pronounce than | Particulates |
| | | | | | | | | Reference Suspension IV | |
| | | 50 mM | — | | T0 | NA | Colorless Liquid | Clear Liquid | Free of visible |
| | | NaCl | | | | | | | Particulates |
| | | | | | 1.5 W | 40° C. | Colorless Liquid | Opalescence is not more | Free of visible |
| | | | | | | | | pronounce than | Particulstes |
| | | | | | | | | Reference Suspension III | |
| | | | | | 3 W | 40° C. | Colorless Liquid | Opalescence is not more | Very Few |
| | | | | | | | | pronounce than | Particulates |
| | | | | | | | | Reference Suspension IV | |
| | | 100 mM | — | | T0 | N/A | Colorless Liquid | Clear Liquid | Free of visible |
| | | NaCl | | | | | | | Particulates |
| | | | | | 1.5 W | 40° C. | Colorless Liquid | Opalescence is not more | Free of visible |
| | | | | | | | | pronounce than | Particulates |
| | | | | | | | | Reference Suspension III | |
| | | | | | 3 W | 40° C. | Colorless Liquid | Opalescence is not more | Very Few |
| | | | | | | | | pronounce than | Particulates |
| | | | | | | | | Reference Suspension IV | |
| 20 mM Na | | — | 0.02 | | T0 | N/A | Colorless Liquid | Clear Liquid | Free of visible |
| Phosphate | | | | | | | | | Particulates |
| | | | | | 1.5 W | 40° C. | Colorless Liquid | Opalescence is not more | Free of visible |
| | | | | | | | | pronounce than | Particulates |
| | | | | | | | | Reference Suspension IV | |
| | | | | | 3 W | 40° C. | Colorless Liquid | Opalescence is not more | Free of visite |
| | | | | | | | | pronounce than | Particulates |
| | | | | | | | | Reference Suspension IV | |
| | | 50 mM | 0.02 | | T0 | N/A | Colorless Liquid | Clear Liquid | Free of visible |
| | | NaCl | | | | | | | Particulates |
| | | | | | 1.5 W | 40° C. | Colorless Liquid | Opalescence is not more | Free of visible |
| | | | | | | | | pronounce than | Particulates |
| | | | | | | | | Reference Suspension IV | |
| | | | | | 3 W | 40°C | Colorless Liquid | Opalescence is not more | Free of visitsle |
| | | | | | | | | pronounce than | Particulates |
| | | | | | | | | Reference Suspension IV | |
| | | 100 mM | 0.02 | | T0 | N/A | Colorless Liquid | Clear Liquid | Free of visible |
| | | NaCl | | | | | | | Particulates |
| | | | | | 1.5 W | 40° C. | Colorless Liquid | Opalescence is not more | Free of visible |
| | | | | | | | | pronounce than | Particulates |
| | | | | | | | | Reference Suspension IV | |
| | | | | | 3 W | 40° C. | Colorless Liquid | Opalescence is not more | Free of visible |
| | | | | | | | | pronounce than | Particulates |
| | | | | | | | | Reference Suspension IV | |

TABLE 33

| | | | | | | | Average |
|---|---|---|---|---|---|---|---|
| Form- | | Excipient | Excipient | PS-80% | Time | | Concentration |
| ulation | Buffer | 1 | 2 | (w/v) | Point | Condition | (mg/mL) |
| F1 | 20 mM Na | 175 mM | — | — | T0 | N/A | 20.4 |
| | Phosphate | Trehalose | | | 3 W | 5° C. | 24.1 |
| | | | | | | 40° C. | 23.2 |
| F2 | | | 50 mM NaCl | | T0 | N/A | 19.7 |
| | | | | | 3 W | 5° C. | 21.6 |
| | | | | | | 40° C. | 22.8 |
| F3 | | | 100 mM NaCl | | T0 | N/A | 23.7 |
| | | | | | 3 W | 40° C. | 22.9 |
| F4 | 50 mM Na | | — | | T0 | NIA | 22.4 |
| | Phosphate | | | | 3 W | 40° C. | 23.6 |
| F5 | | | 50 mM NaCl | | T0 | N/A | 22.5 |
| | | | | | 3 W | 40° C. | 22.7 |
| F6 | | | 100 mM NaCl | | T0 | N/A | 21.7 |
| | | | | | 3 W | 40° C. | 25.2 |
| F7 | 20 mM Na | | — | 0.02 | T0 | N/A | 22.9 |
| | Phosphate | | | | 3 W | 40° C. | 24.2 |
| F8 | | | 50 mM NaCl | 0.02 | T0 | N/A | 22.5 |
| | | | | | 3 W | 40° C. | 23.5 |
| F9 | | | 100 mM NaCl | 0.02 | T0 | N/A | 22.7 |
| | | | | | 3 W | 40° C. | 24.0 |

IEX-HPLC results at the initial time point showed similar profiles with main peak percentages between 64.1% and 66.4%, acidic species percentages between 5.9% and 7.5%, and basic species percentages between 27.7% and 28.7%. After three weeks at 40° ° C. the samples showed significant differences in charge heterogeneity profiles with main peak percentages between 35.0% and 42.3%, acidic species percentages between 22.5% and 32.0%, and basic species percentages between 33.0% and 36.2%. After 7 weeks at 5° ° C. only slight variation was observed with main peak percentages between 59.3% and 61.3%, acidic species percentages between 9.7% and 12.8%, and basic species percentages between 27.9% and 29.0% (Table 34). Formulations with salt have slightly higher main peak percentages, compared to formulations without salt. Addition of 0.02% PS-80 had little to no benefit in terms of charge heterogeneity.

TABLE 34

| | | | | | | | Average | | Average |
|---|---|---|---|---|---|---|---|---|---|
| | | | | PS- | | | Acidic | Average | Basic |
| Form- | | Excipient | Excipient | 80% | Time | | Species | Main | Species |
| ulation | Buffer | 1 | 2 | (w/v) | Point | Condition | (5%) | Peak | (%) |
| F1 | 20 mM Na | 175 mM | — | — | T0 | N/A | 7.48 | 64.11 | 28.42 |
| | Phosphate | Trehalose | | | 3 W | 5° C¹ | 12.76 | 58.30 | 27.94 |
| | | | | | | 40° C. | 26.84 | 38.04 | 35.11 |
| F2 | | | 50 mM | | T0 | N/A | 6.31 | 65.51 | 28.17 |
| | | | NaCl | | 3 W | 5° C.¹ | 9.71 | 61.27 | 29.02 |
| | | | | | | 40° C. | 26.84 | 38.04 | 35.11 |
| F3 | | | 100 MM | | T0 | N/A | 6.06 | 65.96 | 28.08 |
| | | | NaCl | | 3 W | 40° C. | 23.26 | 40.61 | 36.13 |
| F4 | 50 mM Na | | — | | T0 | N/A | 6.33 | 65.68 | 27.99 |
| | Phosphate | | | | 3 W | 40° C. | 22.46 | 41.38 | 36.16 |
| F5 | | | 50 mM | | T0 | N/A | 5.85 | 66.42 | 27.72 |
| | | | NaCl | | 3 W | 40° C. | 25.87 | 40.32 | 33.81 |
| F6 | | | 100 mM | | T0 | N/A | 5.99 | 68.32 | 27.69 |
| | | | NaCl | | 3 W | 40° C. | 24.02 | 41.99 | 33.99 |
| F7 | 20 mM Na | | — | 0.02 | T0 | N/A | 6.42 | 65.37 | 28.21 |
| | Phosphate | | | | 3 W | 40° C. | 24.47 | 42.27 | 33.26 |
| F8 | | | 50 mM | 0.02 | T0 | N/A | 6.11 | 65.42 | 28.48 |
| | | | NaCl | | 3 W | 40° C. | 31.97 | 35.03 | 33.00 |
| F8 | | | 100 mM | 0.02 | T0 | N/A | 6.13 | 65.21 | 28.66 |
| | | | NaCl | | 3 W | 40° C. | 25.40 | 39.03 | 35.56 |

¹Samples were not analyzed until 7 weeks after the study was initiated.

SEC-HPLC results at the initial time point showed similar profiles with main peak percentages between 98.0% and 98.2% and HMW peak percentages between 1.9% and 2.0%. After three weeks at 40° ° C. the samples showed slight degradation in purity due to the appearance of a LMW peak. The main peak percentages for the three week stressed samples were between 94.8% and 95.4%, with HMW peak percentages between 1.9% and 2.1% and LMW peak percentages between 2.7% and 3.1%. After three weeks at 5° C. no significant changes in main peak and HMW peak percentages were observed (Table 35). High and low molecular weight species are minimized with addition of salt, and no benefit was observed when the formulation was supplemented with 0.02% PS-80.

TABLE 35

| | | | | PS- | | | | Main | LMW |
|---|---|---|---|---|---|---|---|---|---|
| Form- | | Excipient | Excipient | 80% | Time | | HMW | Peak | Peak |
| ulation | Buffer | 1 | 2 | (w/v) | Point | Condition | Peak | (%) | (%) |
| F1 | 20 mM Na | 175 mM | — | — | To | N/A | 1.85 | 98.15 | — |
| | Phosphate | Trehalose | | | 3 W | 5° C. | 1.85 | 98.15 | — |
| | | | | | | 40° C. | 2.11 | 94.79 | 3.10 |
| F2 | | | 50 mM | | T0 | NIA | 1.87 | 98.13 | — |
| | | | NaCl | | 3 W | 5° C. | 1.84 | 98.18 | — |
| | | | | | | 40° C. | 1.86 | 95.28 | 2.86 |
| F3 | | | 100 mM | | T0 | N/A | 1.90 | 98.10 | — |
| | | | NaCl | | 3 W | 40°C. | 1.85 | $5.19 | 2.96 |
| F4 | 50 mMA Na | | — | | T0 | N/A | 1.98 | 98.02 | — |
| | Phosphate | | | | 3W | 40° C. | 2.02 | 95.15 | 2.83 |
| F5 | | | 50 mM | | T0 | N/A | 1.97 | 98.03 | — |
| | | | NaCl | | 3 W | 40° C. | 1.98 | 95.28 | 2.75 |
| F6 | | | 100 mM | | T0 | N/A | 1.95 | 98.05 | — |
| | | | NaCl | | 3 W | 40°C. | 1.92 | 95,40 | 2.68 |
| F7 | 20 mM Na | | — | 0.02 | T0 | N/A | 1.93 | $8.07 | — |
| | Phosphate | | | | 3 W | 40° C. | 2.13 | 94.82 | 3.08 |
| F8 | | | 50 mM | 0.02 | T0 | N/A | 1.93 | 98.07 | — |
| | | | NaCl | | 3 W | 40° C. | 1.90 | 95.14 | 2.98 |
| F9 | | | 100 mM | 0.02 | T0 | NA | 1.87 | 98.13 | — |
| | | | NaCl | | 3 W | 40° C. | 1.89 | 95.19 | 2.91 |

### Example 5: Comparison Between Recombinant Crisantaspase Derived from Pseudomonas (RC-P) and Erwinase

Experiments were performed to compare the relevant quality attributes of Recombinant Crisantaspase derived from Pseudomonas (RC-P) and Erwinase. A summary of the data and results are presented in Table 36, comparing the RC-P to Erwinase to demonstrate comparability. The results demonstrate that the RC-P and Erwinase are comparable in their structures and enzymatic activities. Size and charge differences were observed.

TABLE 36
Summary of Comparability Results

| Attribute | Method | RC-P | Erwinase | Results |
|---|---|---|---|---|
| Primary | Peptide | Confirms the expected primary | | Comparable, with |
| Structure | Map LC- - | amino acid sequence, with very | | glycation observed on |
| | MS | consistent PTMs | | lysine for Erwinase |
| Size | Intact Mass | Confirms the expected | | Comparable Monomer |
| | (denatured) | monomer molecular weight | | Molecular weight |
| | | | | measurement, |
| | | | | glycation observed in |
| | | | | Erwinase |
| | SE-HPLC | HMW: 0.2% | HMW: 7.3% | Comparable profiles |
| | | Main: 99.6% | Main: 92.3% | and main contents; |
| | | LMW: 0.1% | LMW: 0.3% | Erwinase has higher |
| | | | | HMW content |
| | SEC- | Tetramer: | Tetramer: | Comparable profiles |
| | MALLS | 99.6% | 93.3% | and tetramer |
| | | | | dominates, Erwinase |
| | | | | has higher octamer |
| | | | | content |
| | SEC-MS | Tetramer: | Tetramer (with | Comparable profiles |
| | (Native) | 99.7% | glycation) | and tetramer dominant, |
| | | | 92.7% | Erwinase has glycation |
| | | | | and higher level of |
| | | | | octamers |
| Charge | CIEX | Acidic: 11% | Acidic: 18% | Comparable profiles |
| | (CIEX-MS) | Main: 84% | Main: 75% | Erwinase has higher |
| | | Basic: 5% | Basic: 7% | acidic variants |
| | iCIEF | pI of main: 8.6 | pI of main: 8.6 | Comparable main peak |
| | | Acidic: 15% | Acidic: 46% | pI Erwinase has higher |
| | | Main: 74% | Main: 45% | acidic variants |
| | | Basic: 11% | Basic: 8% | |
| Hydrophobicity | RP-UHPLC | Prepeak: 14.1% | Prepeak: 17.2% | Comparable profiles |
| | (RP-UHPLC- | Main: 83.2% | Main: 81.2% | and main contents |
| | MS) | Postpeak: 2.7% | Postpeak: 1.5% | |
| | HIC | Main: 98% | Main: 94% | Comparable HIC |
| | | Minor 2% | Minor 2% | profiles and main |
| | | | Late eluting | contents Erwinase has |
| | | | peak: 4% | late eluting peak which |
| | | | | correlate to HMW |
| | | | | species. |
| Higher Order | HDX MS | Very consistent deuterium | | Comparable higher |
| Structure | | uptake maps for RC-P and | | order structures |
| | | Erwinase | | |
| Potency | Nessler | 685 U/mg | 572 U/mg | Comparable |
| | Activity | | | |
| | Assay | | | |

The peptide mapping MS data was mined for peptides containing common or known modifications including oxidation, deamidation, succinimide, isomerization, glycation, methylation and acetylation. The relative extent of each modification was calculated using counts for modified peptides normalized against total peptides, modified and unmodified. The relative level reported here was semi-quantitative. Methylation and acetylation modifications were not found in any of the materials. Oxidation, deamidation and succinimide were found in both RC-P and Erwinase. Their levels were comparable and relatively low as shown in Table 37 (Note: PTMs lower than 0.5% are not reported). In addition, low level of glycations were observed in multiple lysine positions in Erwinase only. Additional glycation modifications in Erwinase would affect its charge properties, resulting in increase of its acidic variants. Overall, both RC-P and Erwinase have confirmed primary amino acid structure. Their PTMs are comparable, except the additional glycation modification observed in Erwinase.

TABLE 37
Observed Post-translational Modifications

| Post-translational Modification | RC-P | Erwinase |
|---|---|---|
| Deamidation % (N281) | 2.0 | 2.3 |
| Oxidation % (M133) | 0.5 | 0.7 |
| Succinimide % (N281) | 3.9 | 4.2 |
| Glycation % (K3 or K53) | ND | 2.0 |
| Glycation % (K219) | ND | 0.8 |
| Glycation % (K269) | ND | 2.4 |
| Glycation % (K318) | ND | 1.7 |

Size-exclusion Chromatography (SE-UHPLC) provides quantitative information about the molecular size distribution of a native protein. The SE-UHPLC profiles for the RC-P and Erwinase showed relative peak area distribution of size-exclusion peaks as listed in Table 38. Both RC-P and Erwinase had dominant main peak content. The amount of high-molecular-weight species (HMW) observed for Erwinase (7.3%) was higher than RC-P (0.2%). The amount of low-molecular-weight species (LMW) observed for both RC-P (0.1%) and Erwinase (0.3%) was comparable.

TABLE 38
Percent HMW, Main Peak and LMW from SE-UHPLC Analysis

| Sample ID/area % | HMW2 | HMW1 | Main Peak | LMW |
|---|---|---|---|---|
| RC-P | ND | 0.2 | 99.6 | 0.1 |
| Erwinase | 1.9 | 5.4 | 92.3 | 0.3 |

Size Exclusion Chromatography with Multi angle Laser Light Scattering: SE-HPLC_MALLS were performed for both RC-P and Erwinase. The identification of size-exclusion peaks was listed in Table 39. SE-HPLC separation combined with MALLS performed on both RC-P and Erwinase identified the SEC main content as tetramer, with a molecular weight in the range of 133-134 kDa. Both materials showed dominant tetramer content. HMW 1 species for both materials was identified as the octamer with a MW in the range of 277-286 kDa, which was close to the theoretical octamer mass of 280 kDa. HMW2 species observed only for Erwinase was identified as the hexadecamer (16-mer) with a MW of 554 kDa, which matched to the theoretical hexadecamer mass of 560 kDa. The size of LMW species could not be accurately assessed by MALLS due to the very low abundance of these forms in both RC-P and Erwinase. SEC-MALLS results showed that both RC-P and Erwinase contained dominant tetramer form, their HMW forms were similar, identified as octamers. In addition, Erwinase contained low level of 16-mer.

TABLE 39
SEC-MALLS Results

| | RC-P | | Erwinase | |
|---|---|---|---|---|
| | Measured | | Measure | |
| Peak/sample/ID | MW Da | Identity | MW Da | Identity |
| HMW2 | NA | ND | 554.1 | Hexadecamer |
| | | | | (16-mer) |
| HMW1 | 277.5 | Octamer | 286.1 | Octamer |
| Main | 133.2 | Tetramer | 133.9 | Tetramer |
| LMW | NA | NA | NA | NA |

Imaged Capillary Isoelectric Focusing (iCIEF): iCIEF is an orthogonal assay to provide quantitative measurement of the charge heterogeneity of RC-P protein. The iCIEF profiles for RC-P and Erwinase, relative peak areas for the main peak, acidic group and basic group with respect to the total area were listed in Table 40. Both materials showed highly comparable pI values for the main content. Erwinase had higher acidic variants content.

TABLE 40
iCIEF Charge Variants Distribution for RC-P and Erwinase

| | | Percent (%) | | |
|---|---|---|---|---|
| | pI | Acidic | | |
| Sample Name | Main Peak | Variants | Main Peak | Basic Variants |
| RC-P | 8.6 | 15 | 74 | 11 |
| Erwinase | 8.6 | 46 | 45 | 8 |

Note:
The total % may not add exactly to 100% due to rounding.

### Oxidation Study:

Both RC-P and Erwinase samples were subjected to forced oxidation by treatment with 0.01% (v/v) H₂O₂ for 4 h at room temperature, respectively. Analytical testing results showed that the oxidized RC-P and Erwinase samples had around 30% oxidation of methionine residues in total, about 20% increase in pre-peak contents of RP-UHPLC, an increase in acidic variants of iCIEF, a slightly increase in HMW content of SEC and had comparable CIEX, HIC, activity properties. Detailed analytical results are discussed below.

Peptide Mapping Results: The tryptic peptide mapping LC-MS methods were employed to confirm the primary structure of the protein and identify potential post translational modifications induced by forced oxidizing conditions, especially methionine oxidations. Peptide mapping results showed that the forced oxidized RC-P and Erwinase had the expected primary structure with oxidization of methionine at multiple locations (Table 41). Overlays of the peptide mapping profiles of H₂O₂ treated RC-P and Erwinase are comparable. Both oxidized sample traces shown an increase peak intensity of oxidized peptide containing M133, M60 and M121 respectively. The H₂O₂ treated RC-P and Erwinase showed different levels of oxidation at multiple methionine locations (Table 41). The methionine locations prone to oxidation from the most to least susceptible were: M133 (˜20%)>M60 (˜8%)>M121(˜4%)>M308(˜1%). Around 33% total methionine oxidation was observed for both samples. Similar levels of deamidation and succinimide intermediates at Asn281 were observed for both oxidized samples and the reference standard.

TABLE 41
PTMs for the Oxidized RC-P and Erwinase

| Post-translational | | | Erwinase |
|---|---|---|---|
| Modification | RC-P RS | RC-P Oxidized | Oxidized |
| Deamidation % (N281) | 2.8 | 2.8 | 3.3 |
| Oxidation % (M133) | 0.5 | 19.5 | 18.5 |
| Oxidation % (M60) | 0.9 | 8.1 | 7.7 |
| Oxidation % (M121) | 0.7 | 4.3 | 4.2 |
| Oxidation % (M308) | 0.6 | 1.2 | 1.1 |
| Succinimide % (N281) | 4.5 | 4.6 | 4.5 |
| Glycation % (K3/K30/ K53) | ND | ND | 3.4 |
| Glycation % (K265/K269) | ND | ND | 2.3 |
| Glycation % (K318) | ND | ND | 1.4 |

SE-UHPLC Results: The SE-UHPLC method was used to evaluate the size distribution of RC-P in its native state under nondenaturing conditions. The overlays of the oxidized RC-P, oxidized Erwinase and reference standard were analyzed. There were no significant profile differences observed between the oxidized samples and the RS. Both oxidized samples showed predominately tetramer peak, a slightly increase of the HMW contents and comparable LMW contents, compared to the reference standard. The relative peak intensities are listed in Table 42.

TABLE 42
SE-UHPLC Results

| Sample ID/area % | HMW1/HMW2 | Main Peak | LMW |
|---|---|---|---|
| RC-P RS (RM-M- | 0.2 | 99.6 | 0.1 |
| 0009) | | | |
| Erwinase | 7.3 | 92.3 | 0.3 |
| Erwinase Oxidized | 11.7 | 88.2 | 0.1 |
| RC-P (GMP4) | 1.2 | 98.7 | 0.1 |
| Oxidized | | | |

CIEX Results: The CIEX method was used to evaluate the charge distribution of RC-P in its native state under non-denaturing conditions. The overlays of the oxidized RC-P, oxidized Erwinase and the reference standard were analyzed. There were no significant profile changes between the oxidized samples and the reference standard. Both of the oxidized samples showed predominately main peak content. The relative peak intensities of the stressed samples and reference standard are listed in Table 43.

TABLE 43
CIEX Results

| Sample ID/area % | Acidic % | Main % | Basic % |
|---|---|---|---|
| RC-P RS (RM-M- | 11 | 85 | 4 |
| 0009) | | | |
| Erwinase | 18 | 75 | 7 |
| Erwinase oxidation | 19 | 72 | 9 |
| stressed | | | |
| RC-P (GMP4) | 12 | 85 | 3 |
| oxidation stressed | | | |

iCIEF Results: The iCHEF method is an orthogonal method to evaluate the charge distribution of RC-P with an applied electrical field. The overlays of the oxidized sample and the reference standard were analyzed. Their relative peak intensities are listed in Table 44. Both oxidized samples showed an increased acidic peak at pI around 8.5. Oxidation have no impact for the basic variants.

TABLE 44
iCIEF Results for the Oxidized Sample and RS

| Sample ID/area % | Acidic % | Main % | Basic % |
|---|---|---|---|
| RC-P RS (RM-M- | 14 | 73 | 12 |
| 0009) | | | |
| Erwinase | 46 | 45 | 8 |
| Erwinase oxidation | 54 | 35 | 11 |
| stressed | | | |
| GMP4 oxidation | 41 | 46 | 14 |
| stressed | | | |

### Low pH Study:

Both RC-P and Erwinase samples were subjected to low pH treatment with 50 mM sodium phosphate at pH 3.4 at room temperature, then frozen at −80° C. Pre-study showed that the incubation time at pH 3.4 was not critical, from 15 min up to 7 days, the SE-UHPLC profiles of treated samples were comparable. Treated samples were relatively stable stored at −80° C. up to 8 weeks. Analytical testing results showed that the treated RC-P and Erwinase samples had around 10% monomer content with reduced activities. Their peptide mapping profiles, PTM, CIEX, RP-UHPLC and HIC properties were comparable to the untreated. Detailed analytical results are discussed below.

SE-UHPLC Results: The SE-UHPLC method is used to evaluate the size distribution of RC-P in its native state under nondenaturing conditions. The overlays of the low pH treated RC-P, Erwinase and reference standard were analyzed. Both low pH treated RC-P and Erwinase showed predominately tetramer peak and around 10% increasing in LMW contents, compared to the reference standard. Low pH treatment led to slightly increase of HMW content for Erwinase from 7.3% to 10.6% while no impact for RC-P. Their relative peak intensities are listed in Table 45.

TABLE 45
SE-UHPLC Results

| Sample ID/area % | HMW1/HMW2 | Main Peak | LMW |
|---|---|---|---|
| RC-P RS (RM-M- | 0.2 | 99.6 | 0.1 |
| 0009) | | | |
| Erwinase | 7.3 | 92.3 | 0.3 |
| Erwinase low pH | 10.6 | 79.9 | 9.5 |
| treated | | | |
| RC-P (GMP4) low | 0.2 | 87.4 | 12.4 |
| pH treated | | | |

CIEX Results: The CIEX method is used to evaluate the charge distribution of RC-P in its native state under non-denaturing conditions. The overlays of the low pH treated RC-P, Erwinase and the reference standard were analyzed. There were no significant profile changes between the pH treated samples and the reference standard. Both of the treated samples showed predominately main peak content. The relative peak intensities of the treated samples and reference standard are listed in Table 46.

TABLE 46
CIEX Results

| Sample ID/area % | Acidic % | Main % | Basic % |
|---|---|---|---|
| RC-P RS (RM-M- | 11 | 85 | 4 |
| 0009) | | | |
| Erwinase | 18 | 75 | 7 |
| Erwinase low pH | 15 | 78 | 7 |
| treated | | | |
| RC-P (GMP4) low | 11 | 85 | 4 |
| pH treated | | | |

iCIEF Results: The iCIEF method is an orthogonal method to evaluate the charge distribution of RC-P with an applied electrical field. The overlays of the low pH treated RC-P, Erwinase and the reference standard were analyzed. Their relative peak intensities are listed in Table 47. Both low pH treated RC-P and Erwinase showed an increased acidic peak at pI around 7.2, which was identified as monomer previously. Low pH treatment had no impact for the basic variants.

TABLE 47
iCIEF Results for the Oxidized Sample and RS

| Sample ID/arca % | Acidic % | Main % | Basic % |
|---|---|---|---|
| RC-P RS (RM-M- | 14 | 73 | 12 |
| 0009) | | | |
| Erwinase | 46 | 45 | 8 |
| Erwinase low pH | 48 | 38 | 13 |
| treated | | | |
| RC-P (GMP4) low | 26 | 59 | 15 |
| pH treated | | | |

Comparability of RC-P and Erwinase was evaluated by comparing their physicochemical, purity, structural and potency characteristics. Size-based heterogeneity, analyzed by SE-UHPLC, SEC-MALLS and SEC-MS, is comparable. Both materials have dominant tetramer structure. The HMW species was identified as an octamer. Erwinase has higher levels of octamer. Low levels of 16-mer species were also observed in Erwinase (<2%). It has previously been demonstrated through size variant characterization of RC-P that the octamer shows comparable activity with tetramer. Overall, the observed size distribution differences between Erwinase and RC-P has minimum impact on drug efficacy. Erwinase has glycation modifications, which contribute to the acidic variants differences. The higher level of octamer/16-mer observed in Erwinase contributed to the late eluting basic variants shown on CIEX. Previous forced degradation study of RC-P demonstrates the glycated RC-P have comparable structure and activity to RC-P. Hydrophobic profiles, as measured by HIC and RP-UHPLC, are comparable for both materials. Minor differences were observed are due to the glycation modification and higher level of HMW content of Erwinase. Primary structure and higher order structure of both materials are comparable. Potency results are comparable. Forced degradation study indicates that Erwinase and RC-P have comparable stability under oxidized and low pH stress. Both oxidized RC-P and Erwinase with around 20% increasing in pre peak of RP-UHPLC have comparable activity compared to their untreated materials. Both RC-P and Erwinase treated with low pH stress condition have reduced activity, which correlates with the monomer contents of the treated samples very well. Based on these data, RC-P demonstrate comparable structure, biological properties and function to Erwinase.

### ENUMERATED EMBODIMENTS

The following enumerated embodiments are representative of some aspects of the inventions.

Embodiment 1: An aqueous, non-lyophilized formulation, comprising:
(i) an L-asparaginase, wherein the L-asparaginase comprises four monomer units, wherein each monomer unit has an amino acid sequence that is at least about 95% identical to SEQ ID NO: 1;
(ii) one or more disaccharides, wherein the one or more disaccharides comprise trebalose, sucrose, or any combination thereof; and
(iii) one or more buffers, wherein the one or more buffers are substantially free of amino acid,
   wherein the formulation comprises less than about 5% low-molecular-weight (LMW) species after storage at 37° ° C. for one week.

Embodiment 2: The formulation of embodiment 1, wherein the L-asparaginase is present at a concentration of about 20 mg/mL.

Embodiment 3: The formulation of embodiment 1 or embodiment 2, wherein the L-asparaginase is non-PEGylated and non-PASylated.

Embodiment 4: The formulation of any one of embodiments 1-3, wherein the one or more disaccharides comprise trehalose.

Embodiment 5: The formulation of embodiment 4, wherein the trehalose is present at a concentration of between about 50 mM and about 300 mM.

Embodiment 6: The formulation of embodiment 4, wherein the trehalose is present at a concentration of between about 150 mM and about 275 mM.

Embodiment 7: The formulation of embodiment 4, wherein the trehalose is present a concentration of about 170 mM.

Embodiment 8: The formulation of any one of embodiments 1-7, wherein the one or more buffers comprise a phosphate buffer, an acetate buffer, or any combination thereof.

Embodiment 9: The formulation of any one of embodiments 1-7, wherein the one or more buffers comprise a phosphate buffer.

Embodiment 10: The formulation of any one of embodiments 1-7, wherein the one or more buffers comprise sodium phosphate.

Embodiment 11: The formulation of embodiment 10, wherein the sodium phosphate is present at a concentration of between about 0.5 mM and about 50 mM.

Embodiment 12: The formulation of embodiment 10, wherein the sodium phosphate is present at a concentration of about 20 mM.

Embodiment 13: The formulation of any one of embodiments 1-12, wherein the formulation further comprises sodium chloride.

Embodiment 14: The formulation of embodiment 13, wherein the sodium chloride is present at a concentration of between about 25 mM and about 150 mM.

Embodiment 15: The formulation of embodiment 13, wherein the sodium chloride is present at a concentration of about 50 mM.

Embodiment 16: The formulation of any one of embodiments 1-15, wherein the formulation further comprises one or more excipients.

Embodiment 17: The formulation of embodiment 16, wherein the one or more excipients comprise polysorbate 80.

Embodiment 18: The formulation of embodiment 17, wherein the polysorbate 80 is present a concentration of between about 0.004% (w/v) and about 0.2% (w/v).

Embodiment 19: The formulation of embodiment 17, wherein the polysorbate 80 is present at a concentration of about 0.02% (w/v).

Embodiment 20: The formulation of any one of embodiments 1-19, wherein the formulation has a pH of between about 4.0 and about 8.5.

Embodiment 21: The formulation of any one of embodiments 1-19, wherein the formulation has a pH of about 7.0.

Embodiment 22: A method of treating a disease, condition, or disorder that is treatable by asparagine depletion in a subject in need thereof, comprising administering to the subject a formulation of any one of embodiments 1-21.

Embodiment 23: The method of embodiment 22, wherein the disease, condition, or disorder is cancer.

Embodiment 24: The method of embodiment 23, wherein the cancer is acute lymphoblastic leukemia (ALL).

Embodiment 25: The method of embodiment 24, wherein the ALL is relapsed ALL.

Embodiment 26: The method of embodiment 23, wherein the cancer is lymphoblastic lymphoma (LBL).

Embodiment 27: The method of embodiment 26, wherein the LBL is relapsed LBL.

Embodiment 28: The method of any one of embodiments 22-27, wherein the formulation is administered intramuscularly.

Embodiment 29: The method of any one of embodiments 22-27, wherein the formulation is administered intravenously.

Embodiment 30: The method of any one of embodiments 22-29, wherein the formulation is co-administered with one or more other chemotherapeutic agents.

Embodiment 31: A kit, comprising: (i) a formulation of any one of embodiments 1-21; and (ii) instructions for treating a disease, condition, or disorder that is treatable by asparagine depletion in a subject in need thereof.

Embodiment 32: The kit of embodiment 31, wherein the disease, condition, or disorder is cancer.

Embodiment 33: The kit of embodiment 32, wherein the cancer is acute lymphoblastic leukemia (ALL).

Embodiment 34: The kit of embodiment 33, wherein the ALL is relapsed ALL.

Embodiment 35: The kit of embodiment 32, wherein the cancer is lymphoblastic lymphoma (LBL).

Embodiment 36: The kit of embodiment 35, wherein the LBL is relapsed LBL.

1. An aqueous, non-lyophilized formulation, comprising:
(i) an L-asparaginase, wherein the L-asparaginase comprises four monomer units, wherein each monomer unit has an amino acid sequence that is at least about 70% identical to SEQ ID NO: 1; and
(ii) one or more stabilizers, or one or more buffers, or any combination thereof.

2. The formulation of claim 1, wherein the one or more stabilizers comprise one or more disaccharides, one or more sorbitols, one or more amino acids, or any combination thereof.

3. The formulation of claim 2, wherein the one or more disaccharides comprise trehalose, sucrose, or any combination thereof.

4. The formulation of claim 1, wherein the one or more buffers are substantially free of amino acid.

5. The formulation of claim 1, wherein the formulation comprises:
a) less than about 0.6% low-molecular-weight (LMW) species after storage at 40° C. for two months;
b) less than about 0.6% LMW species after storage at 37° ° C. for one week;
c) less than 2% high-molecular-weight (HMW) species after storage at 40° C. for two months; or
d) a combination thereof.

6-7. (canceled)

8. The formulation of claim 1, wherein the L-asparaginase is present at a concentration of about 20 mg/mL.

9. The formulation of claim 1, wherein the L-asparaginase is non-PEGylated and non-PASylated.

10. (canceled)

11. The formulation of claim 1, wherein:
a) the one or more buffers comprise a phosphate buffer, an acetate buffer, or any combination thereof;
b) the one or more buffers are present at a concentration of between about 0.5 mM and 50 mM; or
c) a combination thereof.

12. (canceled)

13. The formulation of claim 11, wherein the one or more buffers comprise sodium phosphate.

14-15. (canceled)

16. The formulation of claim 1 wherein the formulation further comprises sodium chloride.

17. The formulation of claim 16, wherein the sodium chloride is present at a concentration of between 25 mM and about 150 mM.

18. The formulation of claim 1, wherein the formulation further comprises one or more excipients.

19. The formulation of claim 18, wherein:
a) the one or more excipients comprise polysorbate 80, polysorbate 20, poloxamer 188, or any combination thereof;
b) the one or more excipients are present at a concentration of between about 0.004% (w/v) and about 0.3% (w/v); or
c) a combination thereof.

20. (canceled)

21. The formulation of claim 1, wherein the formulation has a pH of between about 4.0 and about 8.5.

22. A method of treating a disease, condition, or disorder that is treatable by asparagine depletion in a subject in need thereof, comprising administering to the subject an aqueous, non-lyophilized formulation, comprising:
i) an L-asparaginase, wherein the L-asparaginase comprises four monomer units, wherein each monomer unit has an amino acid sequence that is at least about 70% identical to SEQ ID NO: 1; and
ii) one or more stabilizers, or one or more buffers, or any combination thereof.

23. The method of claim 22, wherein the disease, condition, or disorder is cancer.

24. The method of claim 23, wherein the cancer is acute lymphoblastic leukemia (ALL), relapsed ALL, lymphoblastic lymphoma (LBL), or relapsed LBL.

25-27. (canceled)

28. The method of claim 22, wherein the formulation is administered intramuscularly, intravenously, or subcutaneously.

29-30. (canceled)

31. The method of claim 22, wherein the formulation is co-administered with one or more other chemotherapeutic agents.

32. A kit, comprising:
a) an aqueous, non-lyophilized formulation, comprising:
i) an L-asparaginase, wherein the L-asparaginase comprises four monomer units, wherein each monomer unit has an amino acid sequence that is at least about 70% identical to SEQ ID NO: 1, and
ii) one or more stabilizers, or one or more buffers, or any combination thereof; and
b) instructions for treating a disease, condition, or disorder that is treatable by asparagine depletion in a subject in need thereof.

33-38. (canceled)


SEQUENCE LISTING (1 sequence)

SEQ ID NO: 1 - protein, 327 aa, Artificial Sequence
  Synthetic Construct
ADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY